(19) United States
(12) Patent Application Publication
(10) Pub. No.: US 2026/0217815 A1
(43) Pub. Date: Jul. 30, 2026

(54) TREATMENT OF ATOPIC DERMATITIS WITH MULTISPECIFIC ANTIBODIES

(71) Applicant: NOVARTIS AG, Basel (CH)

(72) Inventors: Bo BANG, Basel (CH); Amita BANSAL, Reinach (CH); Oliver BARENBOLD, Basel (CH); Andrzej Marek BIENCZAK, Basel (CH); Anton DROLLMANN, Allensbach (DE); Motoi HOSOE, Basel (CH); Michael KIFFE, Efringen-Kirchen (DE); Jiri KOVARIK, Zurich (CH); Anubhav Narain MATHUR, San Francisco, CA (US); Patrycja Anna MISIOLEK, Basel (CH); Oliver Martin POHL, Bernex (CH); Eva Christine SCHLOSSER, Basel (CH)

(21) Appl. No.: 19/460,399

(22) Filed: Jan. 27, 2026

Related U.S. Application Data
(60) Provisional application No. 63/751,062, filed on Jan. 29, 2025.

(51) Int. Cl. C07K 16/24; A61K 39/00

(52) U.S. Cl. CPC C07K 16/244; A61K 2039/505; C07K 2317/31; C07K 2317/94

(57) ABSTRACT

The invention relates to therapeutic applications of multispecific antibodies binding IL-18 and IL-13 or of antigen-binding fragments thereof in the treatment and/or prevention of atopic dermatitis or a related condition.

### FIELD

The present invention relates to relates to the treatment and/or prevention of atopic dermatitis or a related condition. More specifically, the invention relates to the administration of a multispecific antibody targeting interleukin 18 (IL-18) and interleukin 13 (IL-13), or an antigen-binding fragment thereof, to treat or prevent atopic dermatitis or a related condition in a subject in need thereof.

### SEQUENCE LISTING

The instant application contains a Sequence Listing which has been submitted electronically in XML format and is hereby incorporated by reference in its entirety. The XML copy, created on Nov. 6, 2025, is named “PAT059884-PCT-SEC01_SQLxml” and is 64,809 bytes in size.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present disclosure are intended.

### BACKGROUND

Atopic dermatitis (AD) is a chronic/relapsing inflammatory skin disease, typically characterized by symptoms including intense pruritus (e.g., severe itch) and by scaly and dry eczematous lesions. Severe disease can be extremely disabling due to major psychological problems, significant sleep loss, and impaired quality of life, leading to high socioeconomic costs. The pathophysiology of AD is influenced by a complex interplay between Immunoglobulin E (IgE)-mediated sensitization, the immune system, and environmental factors. The primary skin defect may be an immunological disturbance that causes IgE-mediated sensitization, with epithelial-barrier dysfunction that is the consequence of both genetic mutations and local inflammation. AD often begins in childhood before age 5 and may persist into adulthood.

Typical treatments for AD include topical lotions and moisturizers, topical corticosteroid ointments, creams or injections. Most treatment options, however, offer only temporary, incomplete, symptom relief. Moreover, many patients with moderate-to-severe AD become resistant to treatment by topical corticosteroids or by calcineurin inhibitors. Recently, a number of biologics have become available for AD treatment. Dupilumab, available under the commercial name Ebglyss®, is a monoclonal anti-IL-4Rα antibody that is approved for the treatment of moderate-to-severe AD, with other biologics such as the anti-IL-13 monoclonal antibodies lebrikizumab (Ebglyss®) and tralokinumab (Adtralza®) also being available. However, currently available biologics-based therapies are characterized by suboptimal efficacies, requirement for inconvenient administration, and/or presence of side-effects. Accordingly, there is still a need for improved therapies for the treatment and/or prevention of atopic dermatitis and its associated symptoms, for therapies with improved efficacies, for therapies allowing more convenient dosing regimens, and for therapies with decreased side-effects. WO2023/209568 describes multispecific, such as bispecific, antibodies binding to IL-18 and IL-13.

### SUMMARY

The present invention provides novel therapies for atopic dermatitis or related conditions comprising administration of a multispecific antibody or an antigen-binding fragment thereof binding to IL-18 and IL-13 (“anti-IL-18/IL-13” multispecific antibody) to a subject in need thereof. The therapies described herein may be associated with improved efficacy, more convenient dosing regimens, and/or decreased side effects relative to therapies available in the art, particularly in the case of moderate-to-severe atopic dermatitis and/or its associated symptoms. Thus, the therapies described herein solve one or more of the current needs in the art.

Accordingly, in an aspect, there is provided a multispecific antibody or antigen-binding fragment thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
for use in the treatment and/or prevention of atopic dermatitis in a subject in need thereof, wherein at least one loading dose and at least one maintenance dose are administered to the subject.

In a further aspect, there is provided a method of treatment and/or prevention of atopic dermatitis in a subject in need thereof, comprising administering at least one loading dose and at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the manufacture of a medicament for the treatment and/or prevention of atopic dermatitis in a subject in need thereof, wherein at least one loading dose and at least one maintenance dose are administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In preferred embodiments, the multispecific antibody or antigen-binding fragment thereof is bispecific. In some embodiments, the first part that binds to IL-18 comprises a first variable heavy region (VH1) comprising SEQ ID NO: 41 and a first variable light region (VL1) comprising SEQ ID NO: 13, and the second part that binds to IL-13 comprises a second variable heavy region (VH2) comprising SEQ ID NO: 55 and a second variable light region (VL2) comprising SEQ ID NO: 27. In some embodiments, the first part that binds to IL-18 comprises a first light chain (LC1) that is a lambda light chain and the second part that binds to IL-13 comprises a second light chain (LC2) that is a kappa light chain. In some embodiments, the first part that binds to IL-18 comprises a first light chain (LC1) comprising SEQ ID NO: 14 and the second part that binds to IL-13 comprises a second light chain (LC2) comprising SEQ ID NO: 28.

In some embodiments, the first part that binds to IL-18 comprises a first Fc polypeptide, and the second part that binds to IL-13 comprises a second Fc polypeptide, wherein the first and/or second Fc polypeptide comprises the amino acid sequence SEQ ID NO: 59 or a variant thereof.

In some embodiments, the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution,
according to EU numbering.

In preferred embodiments,
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution,
according to EU numbering.

In some embodiments, the multispecific antibody or antigen-binding fragment thereof is bispecific and comprises a first part that binds to IL-18 comprising a first heavy chain (HC1) comprising SEQ ID NO: 42 and a first light chain (LC1) comprising SEQ ID NO: 14, and a second part that binds to IL-13 comprising a second heavy chain (HC2) comprising SEQ ID NO: 56 and a second light chain (LC2) comprising SEQ ID NO: 28.

In some embodiments, the multispecific antibody or antigen-binding fragment thereof is bispecific and comprises a first part that binds to IL-18 comprising a first heavy chain (HC1) comprising SEQ ID NO: 57 and a first light chain (LC1) comprising SEQ ID NO: 14, and a second part that binds to IL-13 comprising a second heavy chain (HC2) comprising SEQ ID NO: 58 and a second light chain (LC2) comprising SEQ ID NO: 28.

In preferred embodiments, the IL-18 and IL-13 are human. In preferred embodiments, the subject is a human. In preferred embodiments, the atopic dermatitis is moderate-to-severe atopic dermatitis. In some embodiments, the at least one loading dose is administered subcutaneously or intravenously, preferably subcutaneously. In some embodiments, the at least one maintenance dose is administered subcutaneously or intravenously, preferably subcutaneously. In some embodiments, the at least one loading dose and/or the at least one maintenance dose comprise an amount of the multispecific antibody or antigen-binding fragment thereof sufficient to achieve a therapeutically effective serum level. In some embodiments, the therapeutically effective serum level is maintained throughout the course of the treatment.

In some embodiments, the at least one loading dose comprises an amount of the multispecific antibody or antigen-binding fragment thereof selected from the group consisting of about 60 mg, about 150 mg, about 300 mg, and about 600 mg. In some embodiments, the at least one maintenance dose comprises an amount of the multispecific antibody or antigen-binding fragment thereof selected from the group consisting of about 60 mg, about 150 mg, about 300 mg, and about 600 mg.

In some embodiments, at least two or at least three loading doses are administered. In some embodiments, a first loading dose is administered on day 1, a second loading dose is administered on week 1, and a third loading dose is administered on week 2 of the treatment.

In some embodiments, at least two, at least three, at least four, at least five, at least six, at least seven, at least eight, at least nine, at least ten, at least eleven, or at least twelve maintenance doses are administered. In some embodiments, maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

In some embodiments, at least two maintenance doses are administered, and the first maintenance dose is administered on week 4 of the treatment, followed by administration once every two weeks (Q2W), once every four weeks (Q4W), once every eight weeks (Q8W), or once every twelve weeks (Q12W).

In some embodiments, the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

In some embodiments, at least one, at least two, or at least three (e.g., three) loading doses comprising about 60 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the multispecific antibody or antigen-binding fragment thereof are administered.

In some embodiments, at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 150 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 300 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 600 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, the subject, at baseline, has one or more of the following:
an Eczema Area and Severity Index (EASI) score of ≥12 or ≥16, preferably ≥16,
an Investigator's Global Assessment (IGA) score of ≥3, and/or
a Body Surface Area (BSA) of ≥10%.

In some embodiments, the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI) score;
Peak Pruritus Numeric Rating scale (PP-NRS) score, and;
a combination thereof.

In some embodiments, the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment.

In some embodiments, the treatment results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP).

In some embodiments, the subject has a history of inadequate response to treatment with a topical medication or is a subject for whom topical medications are medically inadvisable. In some embodiments, the topical medication is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist. In some embodiments, the topical corticosteroid is selected from the group consisting of methylprednisolone aceponate, mometasone furoate, fluticasone propionate, betamethasone valerate, triamcinolone, and hydrocortisone butyrate.

In some embodiments, the treatment results in alleviation of a symptom associated with atopic dermatitis relative to baseline, preferably selected from the group consisting of erythema, induration/papulation, excoriations, lichenification, pruritus, dry skin, thickened skin, cracked skin, scaly skin, raw skin, skin sensitivity, swollen skin, scaly and dry eczematous lesions, and a combination thereof.

In some embodiments, the treatment comprises administration of at least one additional therapeutic agent. In some embodiments, the at least one additional therapeutic agent is administered concurrently or sequentially with the multispecific antibody or antigen-binding fragment thereof. In some embodiments, the additional therapeutic agent is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

In some embodiments, the multispecific antibody or antigen-binding fragment thereof is comprised in a pharmaceutical composition, and the pharmaceutical composition optionally comprises one or more pharmaceutically acceptable ingredients. In some embodiments, the pharmaceutical composition is comprised in a kit comprising one or more doses of the pharmaceutical composition, optionally together with instructions for use.

In a further aspect, there is provided a multispecific antibody or antigen-binding fragment thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
for use in the treatment and/or prevention of atopic dermatitis in a subject in need thereof, wherein the subject has received at least one loading dose of the multispecific antibody or antigen-binding fragment thereof, and wherein at least one maintenance dose is administered to the subject.

In a further aspect, there is provided a method of treatment and/or prevention of atopic dermatitis in a subject in need thereof, wherein the subject has received at least one loading dose of the multispecific antibody or antigen-binding fragment thereof, comprising administering at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the manufacture of a medicament for the treatment and/or prevention of atopic dermatitis in a subject in need thereof, wherein the subject has received at least one loading dose of the multispecific antibody or antigen-binding fragment thereof, wherein at least one maintenance dose is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In some embodiments, the multispecific antibody or antigen-binding fragment thereof is bispecific. In some embodiments, the first part that binds to IL-18 comprises a first variable heavy region (VH1) comprising SEQ ID NO: 41 and a first variable light region (VL1) comprising SEQ ID NO: 13, and the second part that binds to IL-13 comprises a second variable heavy region (VH2) comprising SEQ ID NO: 55 and a second variable light region (VL2) comprising SEQ ID NO: 27. In some embodiments, the multispecific antibody or antigen-binding fragment thereof is bispecific and comprises a first part that binds to IL-18 comprising a first heavy chain (HC1) comprising SEQ ID NO: 42 and a first light chain (LC1) comprising SEQ ID NO: 14, and a second part that binds to IL-13 comprising a second heavy chain (HC2) comprising SEQ ID NO: 56 and a second light chain (LC2) comprising SEQ ID NO: 28. In some embodiments, the multispecific antibody or antigen-binding fragment thereof is bispecific and comprises a first part that binds to IL-18 comprising a first heavy chain (HC1) comprising SEQ ID NO: 57 and a first light chain (LC1) comprising SEQ ID NO: 14, and a second part that binds to IL-13 comprising a second heavy chain (HC2) comprising SEQ ID NO: 58 and a second light chain (LC2) comprising SEQ ID NO: 28.

In preferred embodiments, the atopic dermatitis is moderate-to-severe atopic dermatitis. In some embodiments, the at least one maintenance dose is administered subcutaneously or intravenously, preferably subcutaneously. In some embodiments, the at least one maintenance dose comprises an amount of the multispecific antibody or antigen-binding fragment thereof selected from the group consisting of about 60 mg, about 150 mg, about 300 mg, and about 600 mg. In some embodiments, maintenance dose administration frequency is selected from the group consisting of once a week (Q1W), once every two weeks (Q2W), once every four weeks (Q4W), one every eight weeks (Q8W), once every twelve weeks (Q12W), and a combination thereof. In some embodiments, each maintenance dose comprises about 60 mg of the multispecific antibody or antigen-binding fragment thereof. In some embodiments, each maintenance dose comprises about 150 mg of the multispecific antibody or antigen-binding fragment thereof. In some embodiments, each maintenance dose comprises about 300 mg of the multispecific antibody or antigen-binding fragment thereof. In some embodiments, each maintenance dose comprises about 600 mg of the multispecific antibody or antigen-binding fragment thereof. In some embodiments, the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks. In some embodiments, the subject has received at least three loading doses of the multispecific antibody or antigen-binding fragment thereof. In some embodiments, a maintenance dose is administered two weeks, four weeks, eight weeks, or twelve weeks after the last loading dose has been administered to the subject.

In a further aspect, there is provided a method of treatment and/or prevention of atopic dermatitis in a subject in need thereof, comprising administering a multispecific antibody or antigen-binding fragment thereof to the subject, wherein the antibody comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a method of treatment and/or prevention of atopic dermatitis in a subject in need thereof, comprising administering a multispecific antibody or antigen-binding fragment thereof to the subject, wherein the antibody comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a multispecific antibody or antigen-binding fragment thereof for use in the treatment and/or prevention of atopic dermatitis in a subject in need thereof, wherein the antibody comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a multispecific antibody or antigen-binding fragment thereof for use in the treatment and/or prevention of atopic dermatitis in a subject in need thereof, wherein the antibody comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof for the manufacture of a medicament for the treatment and/or prevention of atopic dermatitis in a subject in need thereof, wherein the antibody comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof for the manufacture of a medicament for the treatment and/or prevention of atopic dermatitis in a subject in need thereof, wherein the antibody comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof for the treatment and/or prevention of atopic dermatitis in a subject in need thereof, wherein the antibody comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof for the treatment and/or prevention of atopic dermatitis in a subject in need thereof, wherein the antibody comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28,
and wherein the antibody is administered subcutaneously at a dosing regimen selected from the group consisting of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In preferred embodiments, the multispecific antibody is bispecific.

In some embodiments, the dosing regimen is selected from the group consisting of about 300 mg Q2W, about 300 mg Q4W, about 300 mg Q8W, about 300 mg Q12W, about 600 mg Q2W, about 600 mg Q4W, about 600 mg Q8W, and about 600 mg Q12W. In some embodiments, the dosing regimen is 300 mg Q4W or 600 mg Q4W.

In preferred embodiments, the atopic dermatitis is moderate-to-severe atopic dermatitis.

The following detailed description provides further information on the aspects and embodiments of the invention.

### BRIEF DESCRIPTION OF DRAWINGS

FIG. 1 shows observed mean concentration-time pharmacokinetic profiles (black lines with line type corresponding to treatment arm of the FiH study) and 95% confidence interval (respective dotted grey lines) of anti-IL-18/IL-13 bispecific antibody in serum following administration. Antibody concentrations presented on log-scale.

FIG. 2 shows a comparison of model-predicted anti-IL-18/IL-13 bispecific antibody (SEQ ID NO: 42, 14, 56, 28) concentrations over time at steady state with PK concentrations of anti-IL-13 and anti-IL-18 antibodies. Solid lines correspond to median PK concentrations, shaded areas to 95% prediction interval (PI). “Equivalent” indicates that values were multiplied by 2 to adjust for stoichiometric differences between antibodies. All predictions are for an average individual weighing 70 kg. Concentrations presented on log-scale.

FIG. 3 shows a comparison of model-predicted anti-IL-18/IL-13 bispecific antibody concentrations over time for 60 mg, 150 mg, 300 mg and 600 mg Q4W (s.c.) regimens with and without a loading phase at treatment initiation. Shaded areas correspond to 95% prediction interval (PI). All predictions are for an average individual weighing 70 kg.

FIG. 4 shows predicted anti-IL-18/IL-13 bispecific antibody concentrations over time for 600 mg Q4W s.c. dosing regimen continued treatment, as well as after change of dosing interval at week 16 to Q8W or Q12W. Shaded areas correspond to 95% prediction interval (PI). All predictions are for an average individual weighing 70 kg.

FIG. 5 shows predicted anti-IL-18/IL-13 bispecific antibody concentrations over time for 300 mg Q4W s.c. dosing regimen continued treatment, as well as after change of dosing interval at week 16 to Q8W or Q12W. Shaded areas correspond to 95% prediction interval (PI). All predictions are for an average individual weighing 70 kg.

FIG. 6 shows a Phase II study design, to assess the efficacy and safety of anti-IL-18/IL-13 bispecific antibody in patients with moderate-to-severe atopic dermatitis. For the 60 mg s.c. Q4W and 150 mg s.c. Q4W groups, TP2 shown refers to dosing regimens for patients classified as non-responders after TP1. Patients in the 60 mg s.c. Q4W and 150 mg s.c. Q4W groups that are classified as responders will continue with the same dosing regimen in TP2 as having received in TP1. LD: loading dose.

FIG. 7A shows serum TARC levels relative reduction (in %) from baseline (BL) by Fill study treatment group over time, following anti-IL-18/IL-13 bispecific antibody administration.

FIG. 7B shows serum IFNγ levels relative reduction (in %) from baseline (BL) by FiH study treatment group over time, following anti-IL-18/IL-13 bispecific antibody administration.

FIG. 8 shows serum mean (SD) of IL-18 ratio to baseline (pre-dose) by FiH study treatment group over time, following anti-IL-18/IL-13 bispecific antibody administration.

FIG. 9 shows mean (SD) IFNγ ratio to baseline in ex-vivo stimulated samples by FiH study treatment group. IFNγ was measured by ELISA after samples were stimulated ex-vivo with rhIL-18 and rhIL-12p70. BA refers to the average of all pre-dose assessments.

FIG. 10A shows mean (SD) pSTAT6-F-B (pSTAT6 fluorescence blood (matrix), showing intensity of the signal on monocytes) ratio to baseline in ex-vivo stimulated samples by FiH study treatment group. pSTAT6 was measured by flow cytometry after samples were stimulated ex-vivo with rhIL-13. Data shown represents molecules of equivalent soluble fluorochrome (MESF). BA refers to the average of all pre-dose assessments.

FIG. 10B shows mean (SD) pSTAT6-P-B (pSTAT6 percentage (%) blood (matrix), showing monocyte percentage that expresses pSTAT6) ratio to baseline in ex-vivo stimulated samples by FiH study treatment group. pSTAT6 was measured by flow cytometry after samples were stimulated ex-vivo with rhIL-13. BA refers to the average of all pre-dose assessments.

FIG. 11 shows mean (SD) of total IL-13 (pg/mL) by FiH study treatment group over time, following anti-IL-18/IL-13 bispecific antibody administration. EOS: End of study.

FIG. 12 shows serum IFNγ level mean (SD) of % change in ex-vivo stimulated samples by FiH study treatment group. IFNγ was measured by ELISA after samples were stimulated ex-vivo with rhIL-18 and rhIL-12p70. BA refers to the average of all pre-dose assessments. EOS: End of study.

FIG. 13 shows mean (SD) pSTAT6-P-B (pSTAT6 percentage (%) blood (matrix), showing monocyte percentage that expresses pSTAT6) ratio to baseline in ex-vivo stimulated samples by FiH study treatment group. pSTAT6 was measured by flow cytometry after samples were stimulated ex-vivo with rhIL-13. BA refers to the average of all pre-dose assessments. EOS: End of study.

FIG. 14 shows an exemplary anti-IL-18/IL-13 bispecific antibody according to the disclosure.

### DETAILED DESCRIPTION

Unless defined otherwise, all technical and scientific terms used herein have the same meaning as commonly understood by those of ordinary skill in the art to which this disclosure pertains.

The term “IL-18”, “IL18”, “IL-18 polypeptide”, or “IL-18 protein”, refers to interleukin-18, which is a pro-inflammatory cytokine associated with the induction of Th1 responses, enhanced type I macrophage activation and NK/CD8+ T-cell cytotoxicity (Okamura et al. (1995) Nature; 378:88-91; Yoshimoto et al. (1998) J Immunol; 161 (7): 3400-7; Arend et al. (2008) Immunol Rev.; 223:20-38). IL-18 was originally described in 1989 as interferon-gamma (IFN-γ) inducing factor (IGIF). IL-18 is related to the IL-1 family and is structurally related to IL-1B (Okamura et al. (1995) Nature; 378:88-91). IL-18 is primarily produced by macrophages and T-cells as a precursor protein (pro-IL-18) and secreted as an active protein following cleavage by caspase-1 (Dinarello C A et al (1999) J Allergy Clin Immunol; 103:11-24). In addition to macrophages and T-cells, pro-IL-18 is produced by a wide variety of other cells, including keratinocytes, intestinal epithelial cells, and osteoblasts. Throughout this specification, the term IL-18 encompasses both pro-IL-18 (precursor of mature IL-18 prior to protease cleavage) and mature IL-18 (post protease cleavage) interchangeably unless it is specified that the pro- or mature form is meant. The term “IL-18” refers in particular to human IL-18, e.g., to human IL-18 comprising amino acids 37 to 193 of SEQ ID NO: 62.

IL-18 binds with high affinity and signals through the IL-18 receptor (IL-18R), a heteromeric complex of alpha and beta chains encoded by the genes IL18R1 and IL18RAP, respectively (Torigoe K et al (1997) J Biol Chem; 272 (41): 25737-42). The bioactivity of IL-18 is negatively regulated by the IL-18 binding protein (IL-18BP), a naturally occurring and highly specific inhibitor. This soluble protein forms a complex with free IL-18 preventing its interaction with the IL-18 receptor, thus neutralizing and inhibiting its biological activity (Dinarello C A (2000) Ann Rheum Dis; 59 Suppl 1:117-20). IL-18BP is a constitutively secreted protein with high affinity binding to IL-18. Alternate mRNA splicing variants of IL-18BP result in four isoforms. The prominent ‘a’ isoform is present in the serum of healthy humans at 20-fold molar excess compared with IL-18 (Dinarello and Kaplanski (2005) Expert Rev Clin Immunol, 1 (4), 619-632).

The term “IL-13”, “IL13” “IL-13 polypeptide”, or “IL-13 protein”, refers to interleukin 13, a cytokine involved in regulating inflammatory and immune responses. The human and murine amino acid and nucleic acid sequences can be found in public databases, such as GenBank, UniProt and Swiss-Prot. For example, the amino acid sequence of human IL-13 can be found under UniProt Accession No. P35225 and the nucleotide sequence encoding human IL-13 can be found under Accession No. NM_002188. The term “IL-13” refers in particular to human IL-13.

The anti-IL-18/IL-13 multispecific, e.g., bispecific, antibodies described herein and their antigen-binding fragments preferably specifically bind to IL-18 and IL-13, more preferably to human IL-18 and human IL-13.

As used herein, the singular forms “a,” “an” and “the” include plural references unless the content clearly dictates otherwise. Thus, for example, reference to “an antibody” includes a mixture of two or more such antibodies. Thus, unless stated otherwise, the terms are to be understood as “at least one”.

Unless otherwise specifically stated or clear from context, as used herein, the term “about” in relation to a numerical value is understood as being within the normal tolerance in the art, e.g., within two standard deviations of the mean. Thus, “about” can e.g., be within +/−20%, 15%, 10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “comprise”, “comprises”, “comprising”, and the like, encompasses “including” as well as “consisting of”, e.g., a composition “comprising” X may consist exclusively of X or may include something additional, e.g., X+Y. Similarly, a polypeptide or nucleic acid molecule “comprising” an amino acid sequence or a nucleotide sequence respectively, such as the ones described herein, may consist exclusively of the amino acid sequence or nucleotide sequence, or may include additional amino acid or nucleotide sequences. As used herein, the phrase “consisting essentially of” refers to the genera or species of active pharmaceutical agents included in a method or composition, as well as any excipients inactive for the intended purpose of the method or composition. In some cases, the phrase “consisting essentially of” expressly excludes the inclusion of one or more additional active agents other than an antibody of the present disclosure. In some cases, the phrase “consisting essentially of” expressly excludes the inclusion of one or more additional active agents other than an antibody of the present disclosure and a second co-administered active agent.

The term “binding specificity” as used herein refers to the ability of an individual antibody binding site to react with the greatest affinity to the specified antigenic determinant; e.g., an antibody may bind other antigens besides the specified antigen at a lower affinity (e.g., at a 10×, 100×, 1000× lower affinity).

The term “specifically binds” or “selectively binds”, when used in the context of describing the interaction of an antigen and a multispecific antibody or antigen-binding fragment thereof described herein, refers to a binding reaction that is determinative of the presence of a cognate antigen (IL-18 and/or IL-13) in a heterogeneous population of proteins and other biologics, e.g., in a biological sample (such as a blood, serum, cell, cell lysate, cell supernatant, plasma or tissue sample). Binding may also be called specific when the antibody or antigen-binding fragment thereof binds to IL-18 and IL-13 with a binding affinity that is at least two-fold, at least five-fold, or at least ten-fold greater than its affinity for binding to an antigen other than IL-18 and IL-13, or against the background in an immunoassay. Binding affinity can be measured according to standard methods in the art, for example with surface plasmon resonance using commercially available instruments and protocols (e.g., using a Biacore instrument (Cytiva Life Sciences, Uppsala, Sweden)) or solution equilibrium titration (SET). Other examples of suitable methods include enzyme-linked immunosorbent assay (ELISA), radioimmunoassay (REA), FACS analysis, bioassays (e.g., growth inhibition), and Western Blotting.

The term “KD” or “K_{D}” refers to the dissociation equilibrium constant of a particular antibody-antigen interaction and provides a measurement of an antibody's affinity to its target. Comparison of affinity and/or binding properties between two antibodies can also be established without actually determining the KD value for each antibody, but based on a quantitative measurement of binding (e.g. by ELISA or FACS analysis) that is proportional to KD or on a qualitative measurement of affinity or on an inference of affinity (e.g., in functional assay or in vitro or in vivo assay with respect to the antibody exerting an effect associated with binding to its target).

In some embodiments, “specifically binds to IL-18 and IL-13” is intended to refer to a multispecific (e.g., bispecific) antibody or antigen-binding fragment thereof that binds to IL-18 and IL-13 with a KD, respectively, of 100 nM or less, 10 nM or less, 1 nM or less, 100 pM or less, for example 10 pM or less, in particular as measured by SET.

Preferably, the multispecific (e.g., bispecific) antibodies and antigen-binding fragments thereof described herein do not bind to the IL-18/IL-18 binding protein (IL-18BP) complex, i.e., the multispecific antibodies and antigen-binding fragments thereof preferably specifically bind to “free” IL-18. In some embodiments, “does not bind to the IL-18/IL-18BP” complex refers to the antibody or antigen-binding fragment thereof binding to the IL-18/IL-18BP complex with a KD of 1×10⁻⁵ M or greater.

The term “specifically binds” does not exclude cross-species reactivity. For example, an antibody that “specifically binds” to an antigen from one species may also “specifically bind” to that antigen in one or more other species. Thus, such cross-species reactivity does not itself alter the classification of an antibody as a “specific” binder. In certain embodiments, an antibody that specifically binds to human IL-18 and human IL-13 has cross-species reactivity with IL-18 and/or IL-13 from one or more non-human mammalian species, e.g., a primate species (including but not limited to one or more of Macaca fascicularis, Macaca mulatta, and Macaca nemestrina) or a rodent species, e.g., Mus musculus.

The term “antibody” as used herein refers to a polypeptide (or set of polypeptides) of the immunoglobulin family that is capable of binding an antigen non-covalently, reversibly, and typically, specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The variable regions of both the light (VL) and heavy (VH) chain portions determine antigen recognition and specificity. Conversely, the constant regions of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus of the molecule contains the variable region, and the C-terminus contains the constant region; the CH3 and CL domains comprise the carboxy-terminus of the heavy and light chain, respectively. The constant regions typically have a more conserved amino acid sequence relative to the other portion of the immunoglobulin.

The term “antibody” includes, but is not limited to, monoclonal antibodies, human antibodies, humanized antibodies, camelid antibodies, chimeric antibodies, multispecific, e.g., bispecific antibodies and anti-idiotypic (anti-Id) antibodies (including, e.g., anti-Id antibodies to antibodies described herein), as well as parts, such as antigen-binding fragments, thereof. The antibodies can be of any isotype/class (e.g., IgG, IgE, IgM, IgD, IgA, or IgY) or subclass (e.g., IgG1, IgG2, IgG3, IgG4, IgAQ1, or IgA2). Preferred antibodies are of the IgG1 class. The term “antibody” also encompasses antibody fragments and antibody-like molecules i.e., molecules comprising an antibody or an antigen-binding fragment thereof e.g., a molecule comprising a first half antibody having a typical antibody structure (comprising two polypeptides, the first comprising a light chain, and the second comprising a heavy chain) and a second half antibody having e.g., an scFv-Fc format.

The term “antibody fragment” as used herein refers to one or more portions of an antibody. In some embodiments, these portions are part of the constant domain(s) of an antibody, e.g., fragment crystallizable (Fc), constant (C) domains, etc. In some cases, these portions are antigen-binding fragments that retain the ability of binding an antigen. Thus, the term “antigen-binding fragment” as used herein refers to one or more portions of an antibody that retain the ability to bind to IL-18 and IL-13, preferably specifically. Examples of antigen-binding fragments include, but are not limited to, a Fab fragment (comprising the VL, VH, CL and CH1 domains); a F (ab) 2 fragment, a bivalent fragment comprising two Fab fragments linked by a disulfide bridge at the hinge region; an Fd fragment comprising the VH and CH1 domains; an Fv fragment comprising the VL and VH domains of a single arm of an antibody, a single chain Fv fragment (scFv); an (scFv′) 2 (e.g., produced from scFv containing an additional C terminal cysteine by chemical coupling (Adams et al (1993) Can Res 53:4026-4034 and McCartney et al (1995) Protein Eng, 8:301-314) or by spontaneous site-specific dimerization of scFv containing an unpaired C terminal cysteine residue (Kipriyanov et al (1995) Cell. Bio Phys 26:187-204)); a single domain antibody (dAb) fragment (Ward et al., 1989 Nature 341:544-546), that comprises a VH domain or a VL domain; an isolated complementarity determining region (CDR), or other epitope-binding fragments of an antibody. ScFv-sc-Fv tandems ((scFv)2) may also be produced by linking two scFv units by a third peptide linker, (see Kurucz et al (1995) J Immunol, 154:4576-4582).

Antigen-binding antibody fragments can be incorporated into e.g., single variable domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen binding regions (Zapata et al., (1995) Protein Eng. 8:1057-1062; and U.S. Pat. No. 5,641,870). A single variable domain antibody may e.g. comprise a VH, VL, or VHH (variable region of heavy-chain antibody, e.g., a camelid or shark antibody), and may be present in a format (e.g., homo- or hetero-multimer) with other, different variable regions or variable domains where the other regions or domains are not required for antigen binding by the single chain variable domain.

As used herein, an “Fd” or “Fd region” comprises a VH and a CH1, and, optionally, any portion of an antibody hinge region.

The term “diabody” as used herein refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., (1993) Proc. Natl. Acad. Sci. USA 90:6444-6448.

The term “single chain antibody”, “single chain Fv” or “scFv” refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. ScFvs can be prepared according to methods known in the art (see, for example, Bird et al., (1988) Science 242:423-426 and Huston et al., (1988) Proc. Natl. Acad. Sci. USA 85:5879-5883). The scFv molecules typically comprise a linker (e.g., a Ser-Gly linker). Exemplary suitable linkers are described in, e.g., Holliger et al. (1993), Proc. Natl. Acad. Sci. USA 90:6444-6448; Alfthan et al. (1995), Protein Eng. 8:725-731; Choi et al. (2001), Eur. J. Immunol. 31:94-106; Hu et al. (1996), Cancer Res. 56:3055-3061; Kipriyanov et al. (1999), J. Mol. Biol. 293:41-56, and Roovers et al. (2001), Cancer Immunol.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The positions of the CDRs and framework regions can be determined using various known definitions in the art, e.g., Kabat (“Kabat numbering scheme”), Chothia, IMGT, AbM, and combined definitions (see, e.g., Kabat et al. (1991), “Sequences of Proteins of Immunological Interest,” 5th Ed. Public Health Service, National Institutes of Health, Bethesda, MD; Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J. Mol. Biol., 273:927-748 (1997)). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). Under IMGT, the CDR regions of an antibody can be determined using the program IMGT/DomainGap Align.

Under the Kabat numbering scheme, the CDR amino acid residues in the heavy chain variable domain (VH) are typically numbered 31-35 (HCDR1), 50-65 (HCDR2), and 95-102 (HCDR3); and the CDR amino acid residues in the light chain variable domain (VL) are typically numbered 24-34 (LCDR1), 50-56 (LCDR2), and 89-97 (LCDR3). Under the Chothia numbering scheme, the CDR amino acids in the VH are typically numbered 26-32 (HCDR1), 52-56 (HCDR2), and 95-102 (HCDR3); and the CDR amino acid residues in the VL are typically numbered 26-32 (LCDR1), 50-52 (LCDR2), and 91-96 (LCDR3). In a combined Kabat and Chothia numbering scheme, the CDRs typically correspond to the amino acid residues that are part of a Kabat CDR, a Chothia CDR, or both. For instance, under the combined numbering scheme, the CDRs typically correspond to amino acid residues 26-35 (HCDR1), 50-65 (HCDR2), and 95-102 (HCDR3) in a human VH; and amino acid residues 24-34 (LCDR1), 50-56 (LCDR2), and 89-97 (LCDR3) in a human VL.

As used herein, the term “EU numbering” or “EU numbering scheme” refers to the Eu numbering convention for the constant regions of an antibody, as e.g., described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The term “antibody framework” or “framework region” as used herein refers to the part of the variable domain, either VL or VH, which serves as a scaffold for the antigen binding loops (CDRs) of this variable domain.

The term “part antibody” or “half antibody” refers to a portion of an antibody, antibody fragment, antibody-like molecule or multispecific antibody that comprises an antigen-binding domain. In some embodiments, a part or half antibody refers to a heavy and a light chain pair of, for example, an IgG antibody (e.g., IgG1 antibody). In some embodiments, a part or half antibody comprises a polypeptide comprising a VL and a CL domain and a polypeptide comprising a VH domain, a CH1 domain, a hinge domain, a CH2 domain, and a CH3 domain, wherein said VH and VL domains comprise an antigen-binding domain. In some embodiments, a part or half antibody refers to, e.g. a polypeptide comprising a scFv domain and an Fc polypeptide (comprising a CH2 domain and a CH3 domain, and optionally a hinge region). In some embodiments, a part or half antibody comprises a Fab, and optionally further comprises an Fc polypeptide.

In some embodiments, a first part or half antibody may be paired with another part or half antibody, such that the first part and second will associate, e.g., heterodimerize. This can, for example, be achieved via association of Fc polypeptides comprised by the first and second parts forming an Fc region heterodimer, as described later herein. Advantageously, a first part of half antibody may comprise a lambda (λ) light chain (e.g., a lambda 1 light chain), and the second part of half antibody may comprise a kappa light chain (e.g., a kappa 4 light chain), the presence of the lambda and kappa light chains promoting heterodimerization of the first and second part of half antibodies (e.g., as described in WO2023/209568 or WO2018/057955) to form the multispecific, e.g., bispecific, antibody.

In some embodiments, a first part or half antibody is covalently linked to a second part or half antibody. In some embodiments, either a first half antibody, a second part half antibody, or both a first and second half antibody may be linked to an additional antigen binding domain.

Preferably, a “part antibody” or “half antibody” described herein is a “half IgG antibody” The term “part IgG antibody”, “half IgG antibody” or “half IgG antibody fragment” refers to a heavy and light chain pair of an IgG antibody. In such embodiments, the light chain comprises a VL domain and a CL domain, and the heavy chain comprises a VH domain, a CH1 domain, a hinge domain, a CH2 domain, and a CH3 domain (i.e., an Fd and an Fc), wherein said VL and VH domains comprise an antigen-binding domain. Each part or half IgG antibody described herein preferably comprises an Fc polypeptide as described later herein. As with all polypeptides, an Fc polypeptide contains an N-terminus and a C-terminus, each of which is capable of being linked to an antigen-binding domain. An example of a part or half IgG antibody is a part or half IgG1 antibody.

As used herein, the term “Fc” or “Fc polypeptide” includes a polypeptide comprising a CH2 and a CH3, and, optionally, any portion of an antibody hinge region. An Fc region is comprised of two Fc polypeptides, each comprising a CH2 and a CH3, that dimerize to form the Fc region. In some embodiments, an antibody described herein comprises an Fc polypeptide that is a variant of a human Fc polypeptide, such as a human IgG Fc polypeptide (for example a human IgG1 Fc polypeptide). A variant Fc polypeptide may comprise one or more amino acid modifications (mutations), preferably one or more amino acid substitutions, relative to a reference (“wild-type”) Fc polypeptide, for example an Fc polypeptide comprising the amino acid sequence SEQ ID NO: 59. Examples of mutations include silencing mutations, half-life extension mutations, K-i-H (Knob-in-Hole), and FAE (Fab arm exchange) mutations, discussed later herein.

Each part or half antibody in the cases of multispecific antibodies described herein preferably comprises an Fc polypeptide e.g., a first part antibody binding to IL-18 comprises a heavy chain comprising an Fc polypeptide and a second part antibody binding to IL-13 comprises a heavy chain comprising an Fc polypeptide, allowing for the two Fc polypeptides to dimerize into an Fc region of a multispecific (e.g., bispecific) antibody described herein. The dimerization may be promoted by the inclusion of pair K-i-H (Knob-in-Hole) or FAE (Fab arm exchange) mutations in the Fc polypeptides, discussed later herein.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the term antibody is intended to include derivatized and otherwise modified forms of the antibodies described herein, including immuno-adhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag). One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, III. Examples of derivatized antibodies are antibody conjugates, described in more detail later herein.

The term “monospecific antibody,” as used herein, refers to an antibody that binds to a single target antigen. The term “multispecific antibody” as used herein refers to an antibody that binds to two or more different (distinct) antigens or to an antibody that binds to two or more different (distinct) epitopes on the same antigen. Recognition of each antigen is generally accomplished via an “antigen-binding domain” (e.g., a “IL-18 or IL-13 antigen-binding domain”). In some embodiments, the multispecific binding molecules comprise four polypeptide chains that together comprise a plurality, e.g., two or more, e.g., two, three or four, preferably two, antigen-binding domains that bind two or more different antigens. The term “multispecific” thus includes e.g., bispecific, trispecific, and tetraspecific molecules. Preferably, the multispecific antibodies are bispecific. Bispecific IgG molecules are typically bivalent and possess an asymmetric architecture due to the presence of, at least, different Fv regions. Depending on the method of preparation and the origin of the heavy and light chains, they may further differ in the constant regions of the heavy or light chains (e.g., via the introduction of “matching” (or pair) K-i-H or Fab arm exchange (FAE) mutations in the constant region of the heavy chains). A bispecific antibody is typically able to bind two different antigens or epitopes simultaneously or sequentially.

The term “multivalent antibody” as used herein refers to an antibody that has at least two antigen binding domains. In some embodiments, a multivalent antibody is a bivalent antibody, trivalent antibody or a tetravalent antibody. The term “bivalent antibody” as used herein refers to an antibody that has two antigen-binding domains. The terms “trivalent antibody” and “tetravalent antibody” as used herein refer to an antibody that has three antigen-binding domains, or four antigen-binding domains, respectively. The term “monovalent antibody” means that the antibody has one antigen-binding domain.

The term “monovalent with respect to binding IL-18” as used herein means that the antibody has one IL-18 binding domain. The term “monovalent with respect to binding IL-13” as used herein means that the antibody has one IL-13 binding domain. In some preferred aspects, a bispecific antibody is monovalent with respect to binding IL-18 and monovalent with respect to binding IL-13, i.e., has one IL-18 binding domain and one IL-13 binding domain.

The term “monoclonal antibody” as used herein refers to polypeptides, including antibodies, antibody parts, and antigen-binding fragments, that have substantially identical amino acid sequence and/or are derived from the same genetic source. This term also includes preparations of antibody molecules of single molecular composition. A monoclonal antibody composition displays a single binding specificity and affinity for a particular epitope or antigen. Methods for generation of monoclonal antibodies using phage display technology are known in the art (Proetzel, G., Ebersbach, H. (Eds.) Antibody Methods and Protocols. Humana Press ISBN 978-1-61779-930-3; 2012).

The term “humanized” antibody as used herein, refers to an antibody that retains the reactivity of a non-human antibody or antibody variable domain while being less immunogenic in humans. The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al., (2000) J Mol Biol; 296:57-86). The human antibodies may include amino acid residues not encoded by human sequences (e.g., mutations introduced by random or site-specific mutagenesis in vitro or by somatic mutation in vivo).

The term “co-expression” means that different polypeptides are expressed together in a single host cell, common for all the polypeptides. Co-expression of a multispecific, e.g., bispecific, antibody means that the different parts forming the functional multispecific, e.g., bispecific, antibody are expressed in a single, common, host cell. Co-expression may be achieved by incorporating several expression vectors in the expression host cell, such as one for each of the parts of a bispecific antibody, or by incorporating one expression vector encoding all parts of the multispecific antibody.

The term “Fab-arm” refers to one heavy chain-light chain pair of an antibody.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

A “modification” or “mutation” of an amino acid residue/position, as used herein, refers to a change of a primary amino acid sequence as compared to a starting amino acid sequence, wherein the change results from a sequence alteration involving said amino acid residue/positions. The modification may e.g., be a substitution of the residue (or at said position) with another amino acid (e.g., a conservative or non-conservative substitution), an insertion, or a deletion of a residue. A “variant” or “mutant” of a given sequence comprises at least one amino acid modification relative to said sequence. In some embodiments, the amino acid modification is an amino acid substitution.

In some embodiments, an amino acid modification is a conservative amino acid substitution. For polypeptide sequences, “conservatively modified variants” include individual substitutions, deletions or insertions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosed sequences. The following exemplary eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some embodiments, the term “conservative sequence modifications” or “conservative modifications” is used to refer to amino acid modifications that do not significantly affect or alter the binding characteristics of the antibody containing the amino acid sequence.

The term “half-life” as used herein, refers to the time required for the concentration of an antibody to fall by one-half (Rowland M and Towzer T N: Clinical Pharmacokinetics. Concepts and Applications. Third edition (1995); and Bonate P L and Howard D R (Eds): Pharmacokinetics in Drug Development, Volume 1 (2004)). Further details may also be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2nd Rev. ex edition (1982), which describes pharmacokinetic parameters such as alpha half-life and beta half-life and area under the curve (AUC). Preferably, all pharmacokinetic parameters and values quoted herein are to be read as being values in a human. Preferably, the term “half-life” as used herein refers to “in vivo half-life”, i.e., the half-life to the antibody circulating in the blood or serum of a given mammal, preferably a human.

Multispecific, e.g., bispecific, antibodies and antigen-binding fragments thereof described herein are heterodimers, which means that the first part binding to IL-18 comes from a first antibody (binding to IL-18) and the second part binding to IL-13 comes from a second antibody (binding to IL-13). A “heterodimerization modification” is a modification to one or both parts of the antibodies forming the antibody, intended to facilitate such formation. Examples of heterodimerization modifications are the K-i-H and FAE mutations discussed above, and further described later herein.

As used herein, “identity” refers to the sequence matching between two polypeptides, molecules or between two nucleic acids. When a respective position in both of the two compared sequences is occupied by the same base or amino acid (for instance, if a position in each of two polypeptides is occupied by a lysine), then the respective molecules are identical at that position.

The “percentage identity” or “percent identity” between two sequences is a function of the number of identical positions shared by the sequences, taking into account the number of gaps, and the length of each gap, which need to be introduced for optimal alignment of the two sequences. Generally, a comparison is made when two sequences are aligned to give maximum identity. Such alignment can be provided using, for instance, the method of the Needleman and Wunsch (J. Mol. Biol. (48): 444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. As another example, the percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One suitable set of parameters are a Blossum 62 scoring matrix with a gap penalty of 12, a gap extend penalty of 4, and a frameshift gap penalty of 5.

The percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. Two sequences are “identical” if they have the same sequence of amino acids or nucleotides over the region being compared. Two sequences are “substantially identical” if two sequences have a specified percentage of amino acid residues or nucleotides that are the same (i.e., 80% identity, optionally 85%, 90%, 95%, 98% or 99% identity over a specified region, or, when not specified, over the entire sequence), when compared and aligned for maximum correspondence over a comparison window, or designated region as measured using one of the sequence comparison algorithms discussed herein or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window,” as used herein, includes reference to a segment corresponding to a number of contiguous positions in which a sequence may be compared to a reference sequence of the same number of contiguous positions after the two sequences are optimally aligned. The percent identity is typically calculated by comparing sequences of similar lengths.

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information, and be e.g., run using their default parameters. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Other than percentage of sequence identity noted above, another indication that two nucleic acid sequences or polypeptides are substantially identical may be that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions.

The term “isolated antibody” refers to an antibody that is removed from its original or native environment (e.g., the natural environment if it is naturally occurring). Moreover, an isolated antibody may be substantially free of other cellular material and/or chemicals, for example, an antibody isolated from a cell culture supernatant. Such an antibody may be part of a composition (e.g., pharmaceutical composition), and still be considered isolated in that such a composition is not part of the environment in which it was originally or natively found. Methods of isolation are known in the art and include electrophoretic (e.g., SDS-PAGE, isoelectric focusing, capillary electrophoresis), chromatographic (e.g., ion-exchange, reverse phase HPLC), and filtration methods. In some embodiments, the antibody has been isolated and purified to greater than 95% or than about 95%, greater than 96% or than about 96%, greater than 97% or than about 97%, greater than 98% or than about 98%, or greater than 99% or than about 99% purity as determined by any suitable method in the art (e.g., the electrophoretic or chromatographic methods discussed above; see also Flatman et al., 2007, J Chromatogr B Analyt Technol Biomed Life Sci, 848:79-87). Purity may also be e.g., determined using an intact UPLC-MS mass screening approach, e.g., following protein-A purification, or using an LC-MS approach. Purity may also be e.g., determined using size exclusion chromatography, e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA).

The term “epitope” refers to a site on IL-18 and/or IL-13 to which an antibody of the disclosure interacts with and includes linear, discontinuous, or conformational epitopes. Epitopes can be formed both from contiguous amino acids and/or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Degenerate codon substitutions may e.g., be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994). The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as a retroviral vector, a lentiviral vector, an adenoviral vector, or an adeno-associated viral vector (AAV, e.g., AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, lentiviruses, adenoviruses and adeno-associated viruses), which serve equivalent functions.

The term “host cell” refers to a cell into which a recombinant nucleic acid molecule or expression vector has been introduced, e.g., a nucleic acid molecule or expression vector encoding a multispecific, e.g., bispecific, antibody or part thereof described herein. Such a cell may alternatively be referred to as “recombinant host cell” or “engineered host cell”.

As used herein, the term “protein” refers to any organic compound made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, polypeptides, single chain polypeptides or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule. Unless otherwise indicated, a particular polypeptide sequence also implicitly encompasses conservatively modified variants thereof.

The term “subject in need” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject in need” are used herein interchangeably. In a preferred embodiment, the “subject in need” or “patient” is a human. As used herein, the terms “cyno” or “cynomolgus” refer to the cynomolgus monkey (Macaca fascicularis). The term “subject in need thereof” with respect to the therapies of the invention refers to a subject, preferably a human, that could benefit from administration of a therapy described herein, e.g., that exhibits one or more symptoms or indicia of atopic dermatitis or a related condition and/or that has been diagnosed with atopic dermatitis or a related condition. In some embodiments, the therapies described herein may be used to treat patients that show elevated levels of one or more AD-associated biomarkers (described elsewhere herein). For example, the methods and uses provided herein may comprise administering a multispecific, e.g., bispecific, antibody binding to IL-18 and IL-13 (or antigen-binding fragment thereof) to patients with elevated levels of CCL17 (TARC), IL-13, IL-18, IL-18BP, pSTAT6, and/or IFNγ. In some embodiments, the methods and uses provided herein comprise administering a multispecific, e.g., bispecific, antibody binding to IL-18 and IL-13 (or antigen-binding fragment thereof) to patients with elevated levels of CCL17 (TARC), IL-13, IL-18, IL-18BP, pSTAT6, and/or IFNγ, as compared to a subject not diagnosed with atopic dermatitis or a related condition. In some embodiments, the methods and uses provided herein comprise administering a multispecific, e.g., bispecific, antibody binding to IL-18 and IL-13 (or antigen-binding fragment thereof) to patients with abnormal or aberrant levels of a biomarker selected from the group consisting of CCL17 (TARC), pSTAT6, IFNγ, IL-13 (bioactive), IL-18 (bioactive) (e.g., serum free IL-18), IL-18BP (e.g., serum IL-18BP), IgE (e.g., serum IgE), CCL26 (eotaxin-3), CCL22 (MDC2), hsCRP, CD40, IL-24, and IL-22, as compared to a subject not diagnosed with atopic dermatitis or a related condition.

The terms “therapy”, “treat”, “treating” or “treatment” include therapeutic treatments, prophylactic treatments and applications in which one reduces the risk that a subject will develop a disorder or other risk factor. Treatment does not require the complete curing of a disorder and encompasses the reduction of the symptoms or underlying risk factors. The invention further relates to uses or methods of treatment of AD, e.g., mild, moderate-to-severe, or AD, wherein the treatment comprises treating or alleviating one or more symptoms of AD. Thus, as used herein, the terms “therapy”, “treat”, “treatment” and “treating” refer to the reduction or amelioration of the progression, severity and/or duration of AD, or the amelioration or alleviation of one or more symptoms, suitably of one or more discernible symptoms of AD, resulting from the administration of the multispecific, e.g., bispecific, antibody binding to IL-18 and IL-13 or antigen-binding fragment thereof. In specific embodiments, the terms “therapy”, “treat”, “treatment” and “treating” refer to the amelioration or alleviation of at least one measurable physical parameter of AD, wherein the physical parameter is not necessarily discernible by the patient. Subjects in which the abovementioned reduction, amelioration, or alleviation is obtained can be considered to have a positive response to the treatment. “Prevention” as used in this context refers to methods which aim to prevent the onset of a disease or its symptoms or which delay the onset of a disease or its symptoms.

The term “dose” refers to the quantity of a pharmaceutical composition or multispecific, such as bispecific, antibody (or antigen-binding fragment thereof) described herein, which is administered to a subject.

The term “therapeutically effective” or “therapeutically effective amount” or “therapeutically efficacious dose” means a sufficient amount, e.g. of an antibody or composition described herein, or other pharmaceutically active agent, to provide the desired therapeutic effect (i.e., a reduction in disease activity, reduction in disease progression, reduction in disease signs and/or symptoms, etc.). A therapeutically effective amount is also one in which any toxic or detrimental effects resulting from administration are outweighed by the therapeutically beneficial effects.

In some embodiments, “therapeutically effective amount” may refer to the amount of multispecific, e.g., bispecific, antibody binding to IL-18 and IL-13 (or antigen-binding fragment thereof) described herein, the administration of which achieves a specified result, for example, an improvement in an AD-associated parameter, e.g., a decrease in Investigator's Global Assessment (IGA) score, a decrease in Body Surface Area Involvement of Atopic Dermatitis (BSA) score, a decrease in Eczema Area and Severity Index (EASI) score, a decrease in Dermatology Life Quality Index (DLQI) score, and/or a decrease in Peak Pruritus Numeric Rating scale (PP-NRS) score. Further details on the different score systems are provided in the Examples section. In some embodiments, “therapeutically effective amount” may refer to the amount of multispecific, e.g., bispecific, antibody binding to IL-18 and IL-13 (or antigen-binding fragment thereof) described herein, the administration of which achieves, for example, an improvement in the level of one or more AD-associated biomarkers, e.g., one or more AD-associated biomarkers selected from the group consisting of CCL17 (TARC), pSTAT6, IFNγ, IL-13 (bioactive), IL-18 (bioactive) (e.g., serum free IL-18), and IL-18BP (e.g., serum IL-18BP), as compared to the level prior to treatment with the multispecific antibody or antigen-binding fragment thereof (baseline). In some embodiments, the one or more AD-associated biomarkers are selected from the group consisting of CCL17 (TARC), pSTAT6, IFNγ, IL-13 (bioactive), IL-18 (bioactive) (e.g., serum free IL-18), IL-18BP (e.g., serum IL-18BP), IgE (e.g., serum IgE), CCL26 (eotaxin-3), CCL22 (MDC2), hsCRP, CD40, IL-24, and IL-22.

The term “prophylactically effective” amount means a sufficient amount to provide a desired prophylactic effect. Typically, since a prophylactic dose is used in subjects prior to or at an earlier stage of disease, the prophylactically effective amount will be less than the therapeutically effective amount. Therapeutically effective doses of the multispecific, e.g., bispecific, antibodies (or antigen-binding fragments thereof) of the disclosure are described later herein.

The term “atopic dermatitis”, “AD”, or “eczema”, as used herein, means an inflammatory skin disease characterized by intense pruritus (e.g., severe itch) and by scaly and dry eczematous lesions. The term “atopic dermatitis” or “eczema” includes, but is not limited to, AD (eczema) caused by or associated with epidermal barrier dysfunction, allergy (e.g., skin allergy, allergy to certain foods, pollen, mold, dust mite, animals, etc.), radiation exposure, and/or asthma. The present disclosure encompasses methods to treat patients with mild, moderate-to-severe, or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have 10% or more, 15% or more, or 20% or more of the body's skin surface area (BSA) affected, or at least 10% of skin area in addition to involvement of the eyes, hands and body folds. Patients affected by moderate-to-severe AD also, in general, have (i) an Investigator's Global Assessment (IGA) score of at least 3, for example 3 or 4, (ii) an Eczema Area and Severity Index (EASI) score of at least 10, for example at least 12 or at least 16, and/or (iii) itch. Moderate-to-severe AD is also considered to be present in patients who require frequent treatment with topical corticosteroids. In some embodiments, atopic dermatitis is classified as moderate-to-sever by having a Rajka/Langeland criteria score (e.g., described in Rajka & Langeland, 1989, Acta Derm Venereol Suppl 144:13-14) from 4.5 to 9.

In certain embodiments provided herein are methods to treat both the extrinsic and the intrinsic forms of AD. The extrinsic form of AD is associated with IgE-mediated sensitization and increased levels of Th2 cytokines any typically involves 70% to 80% of patients with AD. The intrinsic form without IgE-mediated sensitization typically involves 20% to 30% of patients with AD; these patients have lower levels of IL-4 and IL-13 than extrinsic AD.

Individuals with atopic dermatitis have an increased risk of developing one or more related conditions, typically related to inflammation. Thus, the term “related condition”, as used herein, refers particularly to conditions including those related to inflammation, such as allergy (e.g., skin allergy, allergy to certain foods, pollen, mold, dust mite, animals, etc.), asthma (such as allergic and non-allergic asthma); infectious disorders, skin infection, cutaneous infection, eczema herpeticum, erysipelas, cellulitis, extra-cutaneous infection, encephalitis, endocarditis, infectious arthropathy, enterocolitis, septicemia, respiratory infection, upper respiratory tract infection, lower respiratory tract infection, lung infection, cardiac infection, brain infection, bone infection, gastrointestinal infection, increased colonization with Staphylococcus aureus in lesional and non-lesional skin, autoimmune disorders, respiratory disorders, neuropsychiatric disorders, musculoskeletal disorders, and cardiovascular disorders associated with atopic dermatitis.

A skin infection, in particular a bacterial skin infection, is common in AD and is in part due to breaks in the skin from very dry, split skin and from scratching the itchy areas. In certain embodiments provided herein are methods to treat AD, which is accompanied by an infection, in particular a skin infection, more particularly a skin superinfection. In certain embodiments provided herein are methods to treat AD related conditions, wherein the related condition is an infection, in particular a skin infection, more particularly a skin superinfection. In certain embodiments, the infection is (i) a bacterial infection, e.g., caused by staphylococcal bacteria, such as Staphylococcus aureus or Staphylococcus epidermidis, or streptococcal bacteria, and/or (ii) a viral infection, e.g., herpes viral infection. The term “superinfection” as used herein refers to a secondary infection superimposed on an already affected lesional tissue. Superinfection complicates skin lesions and/or leads to colonization or infection by bacteria including commensals. Superinfection could be a viral or a bacterial infection, which is in part due to breaks in the skin from very dry, split skin and from scratching the itchy areas.

The terms “pruritus” and “itch”, which are used synonymously herein, refer to a sensation that causes the desire or reflex to scratch the skin. Scratching can be problematic particularly in the case of chronic skin diseases that induce itching, such as atopic dermatitis, because the permanent itching stimulates patients to scratch the affected skin areas constantly and/or excessively, which often leads to skin injuries and further deterioration of the skin surface, as well as sleep deprivation, depression and low quality of life.

Various aspects of the invention are described in further detail in the following sections and subsections.

The skilled person understands that aspects and embodiments described herein relating to methods of treatment and/or prevention of atopic dermatitis or a related condition in a subject in need thereof comprising administration of the multispecific antibody or antigen-binding fragment thereof (or a pharmaceutical composition comprising it) to the subject are also applicable to the use of the multispecific antibody or antigen-binding fragment thereof (or the pharmaceutical composition comprising it) for the manufacture of a medicament for treating and/or preventing atopic dermatitis or a related condition, as well as to the multispecific antibody or antigen-binding fragment thereof (or the pharmaceutical composition comprising it) for use in treating and/or preventing atopic dermatitis or a related condition, and to the use of the multispecific antibody or antigen-binding fragment thereof (or the pharmaceutical composition comprising it) for treating and/or preventing atopic dermatitis or a related condition.

Accordingly, in an aspect, there is provided a method of treatment and/or prevention of atopic dermatitis or a related condition in a subject in need thereof, comprising administering a therapeutically effective amount of a multispecific, preferably bispecific, antibody or antigen-binding fragment thereof, wherein the antibody comprises:
i) a first part that binds to IL-18, and
ii) a second part that binds to IL-13,
to the subject.

In an aspect, there is provided the use of a multispecific, preferably bispecific, antibody, or antigen-binding fragment thereof, wherein the antibody comprises:
i) a first part that binds to IL-18, and
ii) a second part that binds to IL-13,
for the manufacture of a medicament for the treatment and/or prevention of atopic dermatitis or a related condition.

In an aspect, there is provided a multispecific, preferably bispecific, antibody, or antigen-binding fragment thereof, wherein the antibody comprises:
i) a first part that binds to IL-18, and
ii) a second part that binds to IL-13,
for use in the treatment and/or prevention of atopic dermatitis or a related condition.

In an aspect, there is provided the use of a multispecific, preferably bispecific, antibody, or antigen-binding fragment thereof, wherein the antibody comprises:
i) a first part that binds to IL-18, and
ii) a second part that binds to IL-13,
for the treatment and/or prevention of atopic dermatitis or a related condition.

The first part that binds to IL-18 can comprise means for binding to IL-18, for example an IL-18 binding domain, and the second part that binds to IL-13 can comprise means for binding to IL-13, for example an IL-13 binding domain.

It is understood that the therapeutic uses described herein involve administration of a therapeutically effective amount of the multispecific, preferably bispecific, antibody or antigen-binding fragment thereof to a subject in need thereof (patient), preferably to a human.

### Multispecific Antibody and Antigen-Binding Fragments Thereof

Multispecific antibodies and antigen-binding fragments thereof described herein may be multivalent and/or monovalent with respect to binding each antigen. Bispecific antibodies, for example, include but are not limited to bivalent (monovalent with respect to binding each antigen), trivalent (bivalent with respect to binding one antigen and monovalent with respect to binding the other antigen), or tetravalent (bivalent with respect to binding both antigens). In preferred embodiments, the multispecific antibody is bispecific. In further preferred embodiments, the bispecific antibody is bivalent and is monovalent with respect to binding IL-18 and monovalent with respect to binding IL-13. In some embodiments, the multispecific antibody is an IL-18 antagonist. In some embodiments, the multispecific antibody is an IL-18 and IL-13 antagonist.

Non-limiting exemplary multispecific antibody formats include, but are not limited to, bivalent bispecific F (ab) 2 format, bivalent bispecific Fab-scFv format, bivalent bispecific scFv-scFv format, bivalent bispecific IgG format, trivalent bispecific IgG format e.g., a whole antibody molecule specific to one antigen having e.g., a single chain Fab (scFab) linked to the C-terminal end of the molecule specific to another antigen, tetravalent bispecific IgG format, trivalent bispecific scFv3 format, trivalent bispecific (scFv)2-Fab format, trivalent bispecific F(ab)3 format, trivalent trispecific scFv3 format, trivalent trispecific Fab-scFv2 format (e.g., a Fab fragment specific to one antigen C-terminally linked to two scFvs specific to another antigen, one on each chain), trivalent trispecific F(ab)3 format, or trivalent trispecific IgG format, or tetravalent trispecific IgG format. Other exemplary formats incorporate sdAbs or fragments thereof such as a VHH, for example a Fab-VHH or scFv-VHH format. Optionally, the multispecific antibody may be incorporated into an antibody-drug conjugate.

In some embodiments, the multispecific, preferably bispecific, antibody comprises a part antibody, scFv, Fab, or half IgG antibody, or antigen-binding fragments thereof, having binding specificity for IL-18; and one or more of a part antibody, scFv, Fab, or half IgG antibody, or antigen-binding fragments thereof, having binding specificity for IL-13. An anti-IL-18 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-18 antigen-binding domain. An anti-IL-13 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-13 antigen-binding domain.

In preferred embodiments, the multispecific, preferably bispecific, antibody is an IgG1 isotype antibody comprising an anti-IL-18 half antibody paired with an anti-IL-13 half antibody.

Protocols for generating multispecific e.g., bispecific/heterodimeric antibodies are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bispecific structure using a heterobifunctional reagent having an amine-reactive group and a sulfhydryl reactive group as described in, e.g., U.S. Pat. No. 4,433,059; bispecific antibodies generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions (Fd regions) of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multi-specific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VL and VH domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; string of VH domains (or VL domains in family members) connected by peptide linkages with crosslinkable groups at the C-terminus further associated with VL domains to form a series of FVs (or scFvs), as described in, e.g., U.S. Pat. No. 5,864,019; VL and VH domains, scFvs, or Fabs wherein one of the antigens is bound monovalently and one of the antigens is bound bivalently, optionally comprising heterodimeric Fc regions, as described in, e.g., WO2011/028952; and single chain binding polypeptides with both a VL and VH domain linked through a peptide linker are combined into multivalent structures through non-covalent or chemical crosslinking to form, e.g., homobivalent, heterobivalent, trivalent, and tetravalent structures using both scFv or diabody type format, as described in, e.g., U.S. Pat. No. 5,869,620. A multispecific, e.g., bispecific, antibody or antigen-binding fragment thereof binding to IL-18 and IL-13 may preferably be generated as described in WO2023/209568.

The amino acid sequences shown in Table 1 are non-limiting examples of sequences that can be comprised by the anti-IL-18/IL-13 multispecific, preferably bispecific, antibodies described herein, as well as by antigen-binding fragments thereof.

In some embodiments, the multispecific, preferably bispecific, antibody or antigen-binding fragment thereof comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In some embodiments, the multispecific, preferably bispecific, antibody or antigen-binding fragment thereof comprises:
i) a first part that binds to IL-18 comprising:
   an HCDR1 comprising SEQ ID NO: 29;
   an HCDR2 comprising SEQ ID NO: 30;
   an HCDR3 comprising SEQ ID NO: 31;
   an LCDR1 comprising SEQ ID NO: 1;
   an LCDR2 comprising SEQ ID NO: 2;
   an LCDR3 comprising SEQ ID NO: 3, and;
ii) a second part that binds to IL-13 comprising:
   an HCDR1 comprising SEQ ID NO: 43;
   an HCDR2 comprising SEQ ID NO: 44;
   an HCDR3 comprising SEQ ID NO: 45;
   an LCDR1 comprising SEQ ID NO: 15;
   an LCDR2 comprising SEQ ID NO: 16, and;
   an LCDR3 comprising SEQ ID NO: 17.

In preferred embodiments, the multispecific, preferably bispecific, antibody or antigen-binding fragment thereof comprises:
i) a first part that binds to IL-18 comprising:
   an HCDR1 comprising SEQ ID NO: 32;
   an HCDR2 comprising SEQ ID NO: 33;
   an HCDR3 comprising SEQ ID NO: 34;
   an LCDR1 comprising SEQ ID NO: 4;
   an LCDR2 comprising SEQ ID NO: 5;
   an LCDR3 comprising SEQ ID NO: 6, and;
ii) a second part that binds to IL-13 comprising:
   an HCDR1 comprising SEQ ID NO: 46;
   an HCDR2 comprising SEQ ID NO: 47;
   an HCDR3 comprising SEQ ID NO: 48;
   an LCDR1 comprising SEQ ID NO: 18;
   an LCDR2 comprising SEQ ID NO: 19, and;
   an LCDR3 comprising SEQ ID NO: 20.

In some embodiments, the multispecific, preferably bispecific, antibody or antigen-binding fragment thereof comprises:
i) a first part that binds to IL-18 comprising:
   an HCDR1 comprising SEQ ID NO: 35;
   an HCDR2 comprising SEQ ID NO: 36;
   an HCDR3 comprising SEQ ID NO: 37;
   an LCDR1 comprising SEQ ID NO: 7;
   an LCDR2 comprising the amino acid sequence RNN;
   an LCDR3 comprising SEQ ID NO: 9, and;
ii) a second part that binds to IL-13 comprising:
   an HCDR1 comprising SEQ ID NO: 49;
   an HCDR2 comprising SEQ ID NO: 50;
   an HCDR3 comprising SEQ ID NO: 51;
   an LCDR1 comprising SEQ ID NO: 21;
   an LCDR2 comprising amino acid sequence LAS, and;
   an LCDR3 comprising SEQ ID NO: 23.

In some embodiments, the multispecific, preferably bispecific, antibody or antigen-binding fragment thereof comprises:
i) a first part that binds to IL-18 comprising:
   an HCDR1 comprising SEQ ID NO: 38;
   an HCDR2 comprising SEQ ID NO: 39;
   an HCDR3 comprising SEQ ID NO: 40;
   an LCDR1 comprising SEQ ID NO: 10;
   an LCDR2 comprising the amino acid sequence RNN;
   an LCDR3 comprising SEQ ID NO: 12, and;
ii) a second part that binds to IL-13 comprising:
   an HCDR1 comprising SEQ ID NO: 52;
   an HCDR2 comprising SEQ ID NO: 53;
   an HCDR3 comprising SEQ ID NO: 54;
   an LCDR1 comprising SEQ ID NO: 24;
   an LCDR2 comprising amino acid sequence LAS, and;
   an LCDR3 comprising SEQ ID NO: 26.

In some embodiments:
the first part that binds to IL-18 comprises a first variable heavy region (VH1) comprising or consisting of SEQ ID NO: 41, or comprising or consisting of a sequence having at least 80%, at least 85%, at least 90%, at least 95%, at least 96%, at least 97%, at least 98%, or at least 99% sequence identity to SEQ ID NO: 41, and a first variable light region (VL1) comprising or consisting of SEQ ID NO: 13, or comprising or consisting of a sequence having at least 80%, at least 85%, at least 90%, at least 95%, at least 96%, at least 97%, at least 98%, or at least 99% sequence identity to SEQ ID NO: 13, and;
the second part that binds to IL-13 comprises a second variable heavy region (VH2) comprising or consisting of SEQ ID NO: 55, or comprising or consisting of a sequence having at least 80%, at least 85%, at least 90%, at least 95%, at least 96%, at least 97%, at least 98%, or at least 99% sequence identity to SEQ ID NO: 55, and a second variable light region (VL2) comprising or consisting of SEQ ID NO: 27, or comprising or consisting of a sequence having at least 80%, at least 85%, at least 90%, at least 95%, at least 96%, at least 97%, at least 98%, or at least 99% sequence identity to SEQ ID NO: 27.

In some embodiments, the first part that binds to IL-18 comprises a first light chain (LC1) that is a lambda light chain, e.g., a lambda 1 light chain, and the second part that binds to IL-13 comprises a second light chain (LC2) that is a kappa light chain, e.g., a kappa 4 light chain. Preferably, the first light chain (LC1) comprises or consists of SEQ ID NO: 14, or comprises or consists of a sequence having at least 80%, at least 85%, at least 90%, at least 95%, at least 96%, at least 97%, at least 98%, or at least 99% sequence identity to SEQ ID NO: 14, and the second light chain (LC2) comprises or consists of SEQ ID NO: 28, or comprises or consists of a sequence having at least 80%, at least 85%, at least 90%, at least 95%, at least 96%, at least 97%, at least 98%, or at least 99% sequence identity to SEQ ID NO: 28.

In some embodiments, the first part that binds to IL-18 is chimeric, humanized, or human, preferably is human. In some embodiments, the second part that binds to IL-13 is chimeric, humanized, or human, preferably is humanized.

In some embodiments, the multispecific, preferably bispecific, antibody or antigen-binding fragment thereof comprises an Fc region. An exemplary such region can be formed by dimerization of a first Fc polypeptide comprised by the first part that binds to IL-18, and of a second Fc polypeptide comprised by the second part that binds to IL-13 as described herein.

Accordingly, in some embodiments,
the first part that binds to IL-18 comprises a first Fc polypeptide, and
the second part that binds to IL-13 comprises a second Fc polypeptide, wherein the first and/or second Fc polypeptide comprises the amino acid sequence SEQ ID NO: 59 or a variant thereof.

It is understood that the first Fc polypeptide comprised by the first part and the second Fc polypeptide comprised by the second part of a multispecific, preferably bispecific, antibody described herein may differ in at least one amino acid residue. The first and/or second Fc polypeptide comprised by the first and second parts of the antibody may contain modifications (e.g., amino acid substitutions) e.g., to extend half-life, silence effector function, and/or to facilitate dimerization of Fc polypeptides to an Fc region (e.g., by introduction of K-i-H substitutions or FAE substitutions).

The amino acid substitutions described herein can be introduced into the Fc polypeptides using techniques which are known in the art. Typically, the DNA encoding the heavy chain(s) is genetically engineered using standard techniques, e.g., described in Directed Mutagenesis: a Practical Approach (M J McPherson (ed.), 1991, IRL Press). Oligonucleotide-mediated mutagenesis is an exemplary method for preparing substitution variants of the DNA encoding the heavy chains. This technique is known in the art as e.g., described by Adelman et al., (1983) DNA, 2:183.

The skilled person understands that in cases of a multispecific, e.g., a bispecific, antibody comprising a first part binding to IL-18 and a second part binding to IL-13 as described herein, the first part may in some embodiments comprise a heavy chain comprising a “hole” mutation in its constant region and the second part may comprise a heavy chain comprising a “knob” mutation in its constant region, or vice versa (the first part comprising the “knob” mutation and the second part comprising the “hole” mutation). Likewise, in the case of FAE mutations, the first part may in some embodiments comprise a heavy chain comprising a K409R or an F405L mutation in its constant region (EU numbering), and the second part may comprise a heavy chain comprising the respective “matching” (or pair) mutation (F405L or K409R as appropriate) in its constant region.

Knobs-into-holes is a well-known heterodimerization technology used for the manufacture of antibodies, particularly multispecific (e.g., bispecific) antibodies. Introduction of either a “knob” or a “hole” in each antibody, for example in the CH3 region of the heavy chain of each antibody promotes the heterodimerization. The engineered antibodies having a “knob” preferentially partner with the engineered antibodies having a “hole”. This method is particularly effective for the production of IgG, for example IgG1, bispecific antibodies wherein production of a “knob” half IgG antibody preferentially partners with the “hole” half IgG antibody.

As the term is used herein, a “knob” refers to at least one amino acid side chain which projects from the interface of a first polypeptide of the multispecific, preferably bispecific, antibody or antigen-binding fragment thereof comprising a heavy chain constant domain and is therefore positionable in a compensatory “hole” in the interface with a second polypeptide of the multispecific, preferably bispecific, antibody or antigen-binding fragment thereof comprising a heavy chain constant domain so as to stabilize the heterodimerization of polypeptide chains (e.g., of the half antibodies) of multispecific antibodies or antigen-binding fragments thereof over homomultimer formation, for example. The knob may exist in the original interface or may be introduced synthetically (e.g. by altering nucleic acid encoding the interface). Typical import residues for the formation of a knob are generally naturally occurring amino acid residues and are typically selected from arginine (R), phenylalanine (F), tyrosine (Y) and tryptophan (W). Most typical are tryptophan and tyrosine. In some embodiments, the original residue for the formation of the protuberance has a small side chain volume, such as alanine, asparagine, aspartic acid, glycine, serine, threonine or valine. A “hole” refers to at least one amino acid side chain which is recessed from the interface of a second polypeptide of the multispecific, preferably bispecific, antibody or antigen-binding fragment thereof comprising a heavy chain constant domain and therefore accommodates a corresponding knob on the adjacent interfacing surface of a first polypeptide of the multispecific, preferably bispecific, antibody or antigen-binding fragment thereof comprising a heavy chain constant domain. The hole may exist in the original interface or may be introduced synthetically (e.g. by altering nucleic acid encoding the interface). The typical import residues for the formation of a hole are usually naturally occurring amino acid residues and are typically selected from alanine (A), serine(S), threonine (T) and valine (V). Most typical are serine, alanine or threonine. In some embodiments, the original residue for the formation of the hole has a large side chain volume, such as tyrosine, arginine, phenylalanine or tryptophan.

In any of the embodiments described herein, the K-i-H CH3 variant domains may be additionally mutated to introduce a pair of cysteine residues. Without being bound by theory, it is believed that the introduction of a pair of cysteine residues capable of forming a disulfide bond provide stability to the heterodimerized antibody.

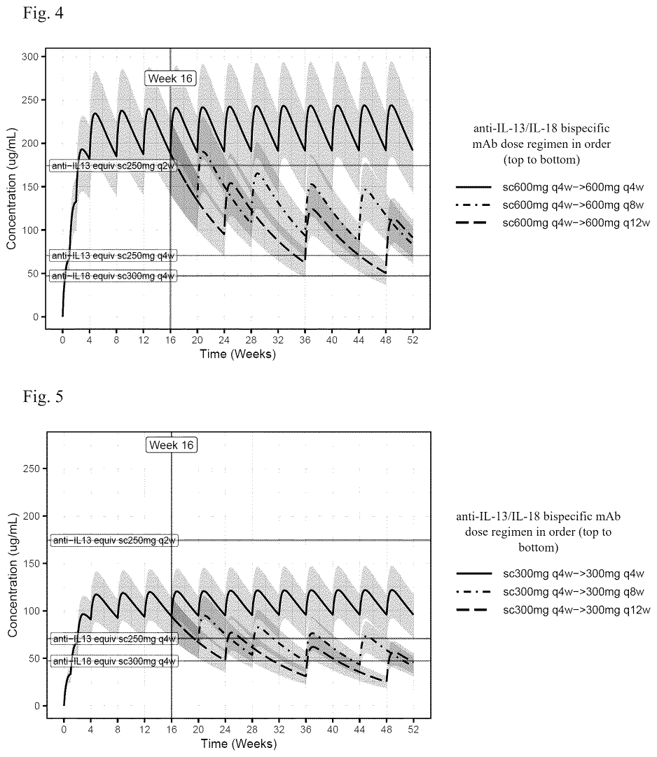

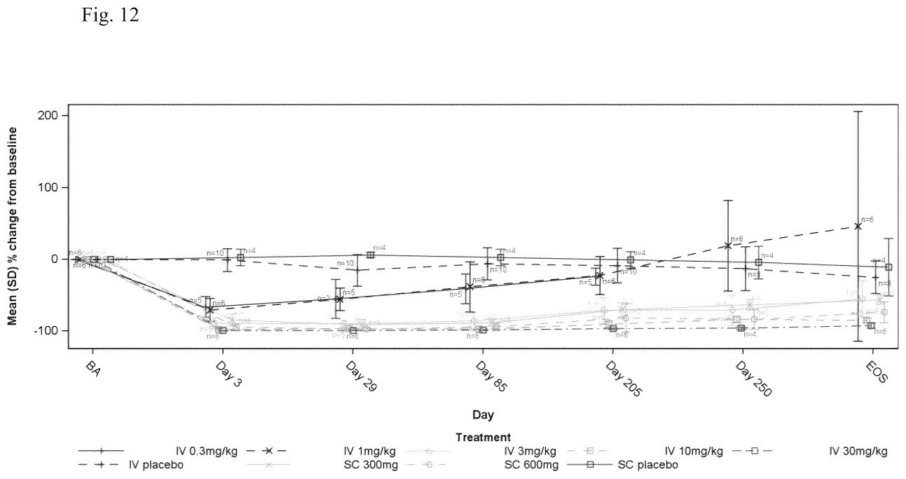

Non-limiting examples of substitutions that can be introduced to generate a knob (e.g., in the part that binds to IL-18 or IL-13) include S354C and/or T366W (EU numbering). Non-limiting examples of substitutions that can be introduced to generate a hole (e.g., in the part that binds to IL-13 or IL-18, depending on the type of substitution that was introduced in the other part) include Y349C, T366S, L368A, and/or Y407V (EU numbering). Additional knob-in-hole substitutions suitable for use in multispecific e.g., bispecific, antibodies or fragments thereof of the present disclosure are described in PCT publication no. WO2014/145806 (for example, FIG. 3, FIG. 4 and FIG. 12 of WO2014/145806), PCT publication no. WO2014/110601, and PCT publication nos. WO2016/086186, WO2016/086189, WO2016/086196, WO2016/182751 and WO1996/027011. Another example of a K-i-H variant comprises a first constant chain comprising a L368D and a K370S mutation, paired with a second constant chain comprising a S364K and E357Q mutation (EU numbering).

Alternatively, heterodimerization may be enhanced by introduction of FAE substitutions, which are particularly suitable when Fab arm exchange is used to obtain the multispecific antibody or antigen-binding fragment thereof. The Fab arm exchange approach is described e.g. by Labrijn et al., Proc Natl Acad Sci USA. 2013 Mar. 26; 110 (13): 5145-50, and involves mixing of two separately expressed parental antibodies under controlled reducing conditions to separate the antibodies into half-antibodies and allow the recombining of half antibodies to form e.g., a bispecific antibody. In an embodiment, the FAE substitutions comprise a K409R substitutions introduced to one CH3 domain paired with F405L substitution in the second CH3 domain, according to the EU numbering scheme, or vice versa. In some embodiments, additional substitutions may also, or alternatively, be introduced at positions selected from the group consisting of positions 366, 368, 370, 399, 405, 407, and 409 according to the EU numbering scheme.

The multispecific antibodies or antigen-binding fragments thereof described herein can be further modified to have an extended half-life in vivo. A variety of strategies known to the skilled person can be used to this end, e.g., chemical linkage to polyethylene glycol (PEG), reCODE PEG, antibody scaffold, polysialic acid (PSA), hydroxyethyl starch (HES), albumin-binding ligands, and carbohydrate shields; genetic fusion to proteins binding to serum proteins, such as albumin, IgG, FcRn, and transferring; coupling (genetically or chemically) to other binding moieties that bind to serum proteins such as nanobodies, Fabs, DARPins, avimers, affibodies, and anticalins; genetic fusion to rPEG, albumin, domain of albumin, albumin-binding proteins, and Fc, or by incorporation into nanocarriers or slow release formulations.

Multispecific antibodies or antigen-binding fragments thereof having an increased half-life in vivo can also be generated introducing one or more amino acid modifications (mutations) (i.e., substitutions, insertions or deletions) into an IgG constant domain, or FcRn binding fragment thereof (preferably a Fc polypeptide or fragment thereof). See, e.g., International Publication No. WO1998/23289; International Publication No. WO1997/34631; WO2013/209568, and U.S. Pat. No. 6,277,375. Half-life extension modifications that may be introduced to the Fc polypeptides of the multispecific preferably bispecific, antibodies or antigen-binding fragments thereof include the “LS” mutation (M428L, N434S, (EU numbering)) and the “YTE” mutation (M252Y, S254T, T256E (EU Numbering)), among which the “YTE” mutation (M252Y, S254T, T256E (EU Numbering)) is preferred.

Further, the multispecific antibodies or antigen-binding fragments thereof can be conjugated or fused to one or more human serum albumin (HSA) polypeptides, or a portion thereof. Albumin can be used to make the molecules more stable in vivo or have a longer half-life in vivo. Such techniques are known in the art, see, e.g., WO1993/15199, WO1993/15200, WO2001/77137, and EP413622. The use of N-terminal fragments of HSA for fusions to polypeptides has also been described (e.g. EP399666). Accordingly, genetically or chemically fusing or conjugating the molecules to albumin can stabilize or extend the shelf-life, and/or retain the molecule's activity for extended periods of time in solution, in vitro and/or in vivo. Additional methods pertaining to HSA fusions can be found, for example, in WO2001/077137 and WO2003/06007. In some embodiments, the expression of the fusion protein is performed in mammalian cell lines, for example, CHO cell lines.

In some cases, it can be beneficial to include or more mutations to silence effector function, e.g., ADCC and/or CDC effector function within the Fc polypeptide of the multispecific, preferably bispecific, antibodies or antigen-binding fragments thereof, referred to herein as “silencing mutations”. Crosslinking of Fc regions via gamma receptors may induce antibody dependent cellular cytotoxicity (ADCC). Human Fc regions when complexed at the cell surface can also bind complement proteins and induce complement dependent cytotoxicity (CDC). Mutations to residues in Fc which reduce or abrogate these interactions may thus limit these effects and focus the impact of the antibodies or fragments thereof described herein upon target cells. More than one mutation, for example amino acid substitutions, can be combined. Non-limiting examples of combinations of amino acid substitutions for antibody effector silencing include the “DAPA” mutation (D265A, P329A in EU numbering) or the “LALA” mutation (L234A, L235A in EU numbering). Other exemplary silencing mutations include the “LALAGA” mutation (L234A, L235A, G237A in EU numbering), the “LALASKPA” mutation (L234A, L235A, S267K, P329A in EU numbering, e.g., as described in WO2022/097065), the “DAPASK” mutation (D265A, P329A, S267K in EU numbering), the “GADAPA” mutation (G237A, D265A, P329A in EU numbering), the “GADAPASK” mutation (G237A, D265A, P329A, S267K in EU numbering), the “LALAPG” mutation (L234A, L235A, P329G in EU numbering), and the “LALAPA” mutation (L234A, L235A, P329A in EU numbering). Further examples of silencing mutations are disclosed in WO2022/097065.

In some embodiments, one or more, e.g., all, of the heavy chain constant region domains of the multispecific, preferably bispecific, antibody or antigen-binding fragment thereof comprise an N279A mutation (according to EU numbering) (see, e.g., Tao M H, Morrison S L. Studies of aglycosylated chimeric mouse-human IgG. Role of carbohydrate in the structure and effector functions mediated by the human IgG constant region. J Immunol. 1989; 143 (8): 2595-601; Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276 (9): 6591-604). Additional Fc mutations for providing silenced effector function are described in WO2014/145806. One example of a silent lgG1 antibody comprises a E233P, L234V, L235A, and S267K mutation, and a deletion of G236 (G236del) (EU numbering). Another example of a silent lgG1 antibody comprises a E233P, L234V, and L235A mutation, and a deletion of G236 (G236del) (EU numbering). Another example of a silent lgG1 antibody comprises a S267K mutation (EU numbering).

Accordingly, in some embodiments, the first and/or second Fc polypeptide comprise a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution, according to EU numbering.

Preferably,
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution, according to EU numbering.

In some embodiments, the multispecific, preferably bispecific, antibody or antigen-binding fragment thereof comprises or consists of:
i) a first part that binds to IL-18 comprising or consisting of a first heavy chain (HC1) comprising or consisting of SEQ ID NO: 42 or 57, or comprising or consisting of a sequence having at least 80%, at least 85%, at least 90%, at least 95%, at least 96%, at least 97%, at least 98%, or at least 99% sequence identity to SEQ ID NO: 42 or 57, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity to SEQ ID NO: 14, and;
ii) a second part that binds to IL-13 comprising or consisting of a second heavy chain (HC2) comprising or consisting of SEQ ID NO: 56 or 58, or comprising or consisting of a sequence having at least 80%, at least 85%, at least 90%, at least 95%, at least 96%, at least 97%, at least 98%, or at least 99% sequence identity to SEQ ID NO: 56 or 58, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof comprises or consists of:
i) a first part that binds to IL-18 comprising or consisting of a first heavy chain (HC1) comprising or consisting of SEQ ID NO: 42, or comprising or consisting of a sequence having at least 80%, at least 85%, at least 90%, at least 95%, at least 96%, at least 97%, at least 98%, or at least 99% sequence identity to SEQ ID NO: 42, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity SEQ ID NO: 14, and;
ii) a second part that binds to IL-13 comprising or consisting of a second heavy chain (HC2) comprising or consisting of SEQ ID NO: 56, or comprising or consisting of a sequence having at least 80%, at least 85%, at least 90%, at least 95%, at least 96%, at least 97%, at least 98%, or at least 99% sequence identity to SEQ ID NO: 56, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof comprises or consists of:
i) a first part that binds to IL-18 comprising or consisting of a first heavy chain (HC1) comprising or consisting of SEQ ID NO: 57, or comprising or consisting of a sequence having at least 80%, at least 85%, at least 90%, at least 95%, at least 96%, at least 97%, at least 98%, or at least 99% sequence identity to SEQ ID NO: 57, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity to SEQ ID NO: 14, and;
ii) a second part that binds to IL-13 comprising or consisting of a second heavy chain (HC2) comprising or consisting of SEQ ID NO: 58, or comprising or consisting of a sequence having at least 80%, at least 85%, at least 90%, at least 95%, at least 96%, at least 97%, at least 98%, or at least 99% sequence identity to SEQ ID NO: 58, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

TABLE 1
Non-limiting exemplary anti-IL-18/IL-13
multispecific antibody sequences.
IL-18 binding part

| | | |
|---|---|---|
| SEQ ID NO: 1 (Combined) | LCDR1 | SGSSSNIGNHYVN |
| SEQ ID NO: 2 (Combined) | LCDR2 | RNNHRPS |
| SEQ ID NO: 3 (Combined) | LCDR3 | QSWDYSGFSTV |
| SEQ ID NO: 4 (Kabat) | LCDR1 | SGSSSNIGNHYVN |
| SEQ ID NO: 5 (Kabat) | LCDR2 | RNNHRPS |
| SEQ ID NO: 6 (Kabat) | LCDR3 | QSWDYSGFSTV |
| SEQ ID NO: 7 (Chothia) | LCDR1 | SSSNIGNHY |
| | LCDR2 | RNN |
| SEQ ID NO: 9 (Chothia) | LCDR3 | WDYSGFST |
| SEQ ID NO: 10 (IMGT) | LCDR1 | SSNIGNHY |
| | LCDR2 | RNN |
| SEQ ID NO: 12 (IMGT) | LCDR3 | QSWDYSGFSTV |
| SEQ ID NO: 13 | VL | DIVLTQPPSVSGAPGQRVTISCSGSSSNIGNHYVN |
| | | WYQQLPGTAPKLLIYRNNHRPSGVPDRESGSKS |
| | | GTSASLAITGLQSEDEADYYCQSWDYSGFSTVF |
| | | GGGTKLTVL |
| SEQ ID NO: 14 | Light Chain 1 | DIVLTQPPSVSGAPGQRVTISCSGSSSNIGNHYVN |
| | | WYQQLPGTAPKLLIYRNNHRPSGVPDRESGSKS |
| | | GTSASLAITGLQSEDEADYYCQSWDYSGFSTVF |
| | | GGGTKLTVLGQPKAAPSVTLFPPSSEELQANKAT |
| | | LVCLISDFYPGAVTVAWKADSSPVKAGVETTTP |
| | | SKQSNNKYAASSYLSLTPEQWKSHRSYSCQVTH |
| | | EGSTVEKTVAPTECS |
| SEQ ID NO: 29 | HCDR1 | GGTFKSYAIS |
| (Combined) | | |
| SEQ ID NO: 30 | HCDR2 | NIIPMTGQTYYAQKFQG |
| (Combined) | | |
| SEQ ID NO: 31 | HCDR3 | AAYHPLVFDN |
| (Combined) | | |
| SEQ ID NO: 32 (Kabat) | HCDR1 | SYAIS |
| SEQ ID NO: 33 (Kabat) | HCDR2 | NIIPMTGQTYYAQKFQG |
| SEQ ID NO: 34 (Kabat) | HCDR3 | AAYHPLVFDN |
| SEQ ID NO: 35 (Chothia) | HCDR1 | GGTFKSY |
| SEQ ID NO: 36 (Chothia) | HCDR2 | IPMTGQ |
| SEQ ID NO: 37 (Chothia) | HCDR3 | AAYHPLVFDN |
| SEQ ID NO: 38 (IMGT) | HCDR1 | GGTFKSYA |
| SEQ ID NO: 39 (IMGT) | HCDR2 | IIPMTGQT |
| SEQ ID NO: 40 (IMGT) | HCDR3 | ARAAYHPLVEDN |
| SEQ ID NO: 41 | VH | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSS |
| SEQ ID NO: 42 | Heavy Chain 1 | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKRVEPKSCDKTHTCPPCP |
| | | APELLGGPSVFLFPPKPKDTLYITREPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVCTLPPSREEMTKNQV |
| | | SLSCAVKGFYPSDIAVEWESNGQPENNYKTTPPV |
| | | LDSDGSFFLVSKLTVDKSRWQQGNVFSCSVMHE |
| | | ALHNHYTQKSLSLSPGK |
| SEQ ID NO: 57 | Heavy chain 1 | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKRVEPKSCDKTHTCPPCP |
| | | APELLGGPSVFLFPPKPKDTLYITREPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVYTLPPCREEMTKNQV |
| | | SLWCLVKGFYPSDIAVEWESNGQPENNYKTTPP |
| | | VLDSDGSFFLYSKLTVDKSRWQQGNVFSCSVMH |
| | | EALHNHYTQKSLSLSPGK |

IL-13 binding part

| | | |
|---|---|---|
| SEQ ID NO: 15 | LCDR1 | RASKSVDSYGNSFMH |
| (Combined) | | |
| SEQ ID NO: 16 | LCDR2 | LASNLES |
| (Combined) | | |
| SEQ ID NO: 17 | LCDR3 | QQNNEDPRT |
| (Combined) | | |
| SEQ ID NO: 18 (Kabat) | LCDR1 | RASKSVDSYGNSFMH |
| SEQ ID NO: 19 (Kabat) | LCDR2 | LASNLES |
| SEQ ID NO: 20 (Kabat) | LCDR3 | QQNNEDPRT |
| SEQ ID NO: 21 (Chothia) | LCDR1 | SKSVDSYGNSF |
| | LCDR2 | LAS |
| SEQ ID NO: 23 (Chothia) | LCDR3 | NNEDPR |
| SEQ ID NO: 24 (IMGT) | LCDR1 | KSVDSYGNSF |
| | LCDR2 | LAS |
| SEQ ID NO: 26 (IMGT) | LCDR3 | QQNNEDPRT |
| SEQ ID NO: 27 | VL | DIVMTQSPDSLSVSLGERATINCRASKSVDSYGN |
| | | SFMHWYQQKPGQPPKLLIYLASNLESGVPDRFS |
| | | GSGSGTDFTLTISSLQAEDVAVYYCQQNNEDPRT |
| | | FGGGTKVEIK |
| SEQ ID NO: 28 | Light Chain 2 | DIVMTQSPDSLSVSLGERATINCRASKSVDSYGN |
| | | SFMHWYQQKPGQPPKLLIYLASNLESGVPDRFS |
| | | GSGSGTDFTLTISSLQAEDVAVYYCQQNNEDPRT |
| | | FGGGTKVEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |
| SEQ ID NO: 43 | HCDR1 | GFSLSAYSVN |
| (Combined) | | |
| SEQ ID NO: 44 | HCDR2 | MIWGDGKIVYNSALKS |
| (Combined) | | |
| SEQ ID NO: 45 | HCDR3 | DGYYPYAMDN |
| (Combined) | | |
| SEQ ID NO: 46 (Kabat) | HCDR1 | AYSVN |
| SEQ ID NO: 47 (Kabat) | HCDR2 | MIWGDGKIVYNSALKS |
| SEQ ID NO: 48 (Kabat) | HCDR3 | DGYYPYAMDN |
| SEQ ID NO: 49 (Chothia) | HCDR1 | GFSLSAY |
| SEQ ID NO: 50 (Chothia) | HCDR2 | WGDGK |
| SEQ ID NO: 51 (Chothia) | HCDR3 | DGYYPYAMDN |
| SEQ ID NO: 52 (IMGT) | HCDR1 | GFSLSAYS |
| SEQ ID NO: 53 (IMGT) | HCDR2 | IWGDGKI |
| SEQ ID NO: 54 (IMGT) | HCDR3 | AGDGYYPYAMDN |
| SEQ ID NO: 55 | VH | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSS |
| SEQ ID NO: 56 | Heavy Chain 2 | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQTYI |
| | | CNVNHKPSNTKVDKRVEPKSCDKTHTCPPCPAP |
| | | ELLGGPSVFLFPPKPKDTLYITREPEVTCVVVDVS |
| | | HEDPEVKFNWYVDGVEVHNAKTKPREEQYNST |
| | | YRVVSVLTVLHQDWLNGKEYKCKVSNKALPAP |
| | | IEKTISKAKGQPREPQVYTLPPCREEMTKNQVSL |
| | | WCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID NO: 58 | Heavy chain 2 | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQTYI |
| | | CNVNHKPSNTKVDKRVEPKSCDKTHTCPPCPAP |
| | | ELLGGPSVFLFPPKPKDTLYITREPEVTCVVVDVS |
| | | HEDPEVKFNWYVDGVEVHNAKTKPREEQYNST |
| | | YRVVSVLTVLHQDWLNGKEYKCKVSNKALPAP |
| | | IEKTISKAKGQPREPQVCTLPPSREEMTKNQVSLS |
| | | CAVKGFYPSDIAVEWESNGQPENNYKTTPPVLD |
| | | SDGSFFLVSKLTVDKSRWQQGNVFSCSVMHEAL |
| | | HNHYTQKSLSLSPGK |
| SEQ ID NO: 59 | Fc | DKTHTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | SREEMTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| SEQ ID NO: 60 | Fc-hole | DKTHTCPPCPAPELLGGPSVFLFPPKPKDTLYITR |
| | | EPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVCTLPPS |
| | | REEMTKNQVSLSCAVKGFYPSDIAVEWESNGQP |
| | | ENNYKTTPPVLDSDGSFFLVSKLTVDKSRWQQG |
| | | NVFSCSVMHEALHNHYTQKSLSLSPGK |
| SEQ ID NO: 61 | Fc-key | DKTHTCPPCPAPELLGGPSVFLFPPKPKDTLYITR |
| | | EPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | CREEMTKNQVSLWCLVKGFYPSDIAVEWESNG |
| | | QPENNYKTTPPVLDSDGSFFLYSKLTVDKSRWQ |
| | | QGNVFSCSVMHEALHNHYTQKSLSLSPGK |

Amino acid sequence variants of the antibodies described herein can e.g., be prepared by introducing appropriate nucleotide changes into their encoding nucleic acid molecules, or by synthesis of the desired variants. Such variants include, for example, variants arising from deletions, insertions, or substitutions, of residues within the amino acid sequences of present antibodies. Any suitable combination of deletions, insertions, and/or substitutions can be made to arrive at a variant construct, provided that the final construct possesses the desired antigen-binding characteristics. The amino acid changes also may alter post-translational processes of the antibodies, such as changing the number or position of glycosylation sites. The present application further includes variants of the antibodies described herein and/or fragments thereof having amino acid conservative modifications in variable regions and/or constant regions.

In some embodiments, a multispecific, preferably bispecific, antibody described herein or antigen-binding fragment thereof comprises an HCDR1, HCDR2, HCDR3, LCDR1, LCDR2, and/or LCDR3 region with a sequence shown in Table 1, or an HCDR1, HCDR2, HCDR3, LCDR1, LCDR2, and/or LCDR3 region having at least 80%, at least 85%, at least 90%, at least 95%, at least 96%, at least 97%, at least 98%, or at least 99% sequence identity with the sequence of an HCDR1, HCDR2, HCDR3, LCDR1, LCDR2, and/or LCDR3 region shown in Table 1. In some embodiments, a multispecific, preferably bispecific, antibody or antigen-binding fragment thereof described herein comprises an HCDR1, HCDR2, HCDR3, LCDR1, LCDR2, and/or LCDR3 region having 1, 2, or 3 substitutions, deletions, or insertions relative to the sequence of an HCDR1, HCDR2, HCDR3, LCDR1, LCDR2, and/or LCDR3 region shown in Table 1. In some embodiments, a multispecific, preferably bispecific, antibody or antigen-binding fragment thereof described herein comprises a VH and/or VL region with a sequence shown in Table 1, or a VH and/or VL region having at least 80%, at least 85%, at least 90%, at least 95%, at least 96%, at least 97%, at least 98%, or at least 99% sequence identity with the sequence of a VH and/or VL region shown in Table 1. In some embodiments, a multispecific, preferably bispecific, antibody or antigen-binding fragment thereof described herein comprises a VL and/or VH region having 1, 2, or 3 substitutions, deletions, or insertions relative to the sequence of a VL and/or VH region shown in Table 1. In some embodiments, a multispecific, preferably bispecific, antibody or antigen-binding fragment thereof described herein comprises a heavy chain and/or light chain with a sequence shown in Table 1, or a heavy chain and/or light chain having at least 80%, at least 85%, at least 90%, at least 95%, at least 96%, at least 97%, at least 98%, or at least 99% sequence identity with the sequence of a heavy chain and/or light chain shown in Table 1. In some embodiments, a multispecific, preferably bispecific, antibody or antigen-binding fragment thereof described herein comprises a heavy chain and/or light chain having 1, 2, or 3 substitutions, deletions, or insertions relative to the sequence of a heavy chain and/or light chain shown in Table 1. In some embodiments, the substitutions are conservative substitutions, as described earlier herein.

In some embodiments, a multispecific, preferably bispecific, antibody or antigen-binding fragment thereof comprises a first part that binds to IL-18 and a second part that binds to IL-13 as described in WO2023/209568.

### Pharmaceutical Compositions

It is understood that the multispecific, preferably bispecific, antibody or antigen-binding fragment thereof described herein may be administered to the subject in need thereof as the active ingredient comprised in a pharmaceutical composition, optionally in combination with one or more pharmaceutically acceptable ingredients. A pharmaceutical composition may be formulated to be suitable for a particular mode of administration. Suitable administration modes are described later herein. The types of formulations, as well as formulation methods, that will be the most appropriate for a pharmaceutical composition described herein for it to be suitable for a particular mode of administration are well within the capabilities of the skilled person and are described in standard handbooks, such as Remington: The Science and Practice of Pharmacy, 23^{rd} edition, Elsevier (2020).

The phrase “pharmaceutically acceptable” means approved by a regulatory agency of a federal or a state government, or listed in the U.S. Pharmacopeia or other generally recognized pharmacopeia for use in animals, and more particularly, in humans. Pharmaceutically acceptable ingredients include pharmaceutically acceptable carriers, fillers, stabilizers, preservatives, solubilizers, vehicles, diluents and/or excipients. Such pharmaceutically acceptable carriers, fillers, stabilizers, preservatives, solubilizers, vehicles, diluents and/or excipients may for instance also be found in Remington: The Science and Practice of Pharmacy (supra).

Pharmaceutical compositions can be prepared in accordance with methods well known and routinely practiced in the art in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy (supra), Pharmaceutical Dosage Forms: Parenteral Medications, Marcel Dekker, NY; Lieberman, et al. (eds.) (1990), Pharmaceutical Dosage Forms: Tablets, Marcel Dekker, NY; Lieberman, et al. (eds.) (1990), Pharmaceutical Dosage Forms: Disperse Systems, Marcel Dekker, NY, Weiner and Kotkoskie (2000) Excipient Toxicity and Safety, Marcel Dekker, Inc., New York, N.Y.). Pharmaceutical compositions are preferably manufactured under GMP conditions.

The pharmaceutical compositions of the invention comprise a therapeutically effective dose or efficacious dose of the multispecific, preferably bispecific, antibody or antigen-binding fragment thereof described herein. More information on preferred dosage regimens is provided later herein.

A pharmaceutical composition described herein can be administered e.g., systemically by methods known in the art. The route and/or mode of administration can vary depending upon the desired results. It is preferred that the composition is a solution or suspension for injection. The composition may e.g., be an aqueous solution for injection comprising one or more pharmaceutically acceptable excipients, diluents or carriers. Such antibody formulations may comprise one or more of an excipient to adjust tonicity and/or osmolality, a buffer and/or a surfactant. The composition should be sterile and fluid. Antibody formulations are known in the art and, for example, include lebrikizumab formulations, marketed as Ebglyss®, which comprise as excipients histidine, glacial acetic acid, sucrose, polysorbate 20 and water for injection.

In some embodiments, the multispecific, preferably bispecific, antibodies or antigen-binding fragments thereof may be co-administered to a patient using a pharmaceutical composition comprising an antibody or antigen-binding fragment thereof in combination with one or more other therapies or therapeutic agents (e.g., a corticosteroid and/or other therapeutic agent). In some embodiments, the pharmaceutical composition further comprises one or more additional therapeutic agents. More information on additional therapeutic agents is provided later herein.

Where necessary, the antibody or fragment thereof may be formulated in a pharmaceutical composition suitable for injection that includes a solubilizing agent and a local anaesthetic such as lidocaine to ease pain at the site of the injection.

The multispecific antibodies or antigen-binding fragments thereof (or pharmaceutical compositions comprising them) described herein can be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal, or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a pharmaceutical composition of the present disclosure can be administered via a non-parenteral route, such as a topical, epidermal or mucosal route of administration, for example, intranasally, orally, vaginally, rectally, sublingually, or topically. Preferred routes of administration are subcutaneous or intravenous administration, with subcutaneous administration being more preferred. A non-limiting example of subcutaneous administration is subcutaneous injection.

The multispecific antibodies or antigen-binding fragments thereof (or pharmaceutical compositions comprising them) described herein may be administered via any of the abovementioned routes using, e.g., an injection device, an injection pen, a vial and syringe, pre-filled syringe, autoinjector, an infusion pump, a patch pump, an infusion bag and needle, etc. If administered in a controlled release or sustained release system, a pump may be used to achieve controlled or sustained release (see e.g., Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neural. 25:351; Howard et al., 1989, J. Neurosurg. 7 1:105); U.S. Pat. Nos. 5,679,377; 5,916,597; 5,912,015; 5,989,463; 5,128,326; WO 1999/15154; and WO 1999/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising an antibody or fragment thereof (see, e.g., U.S. Pat. No. 4,526,938, WO 1991/05548, WO 1996/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'I. Symp. Control. Rel. Bioact. Mater. 24:853-854, and Lam et al., 1997, “Microencapsulation of Recombinant Humanized Monoclonal Antibody for Local Delivery,” Proc. Int'I. Symp. Control Rel. Bioact. Mater. 24:759-760.

If the antibodies or antigen-binding fragments thereof are administered topically, they can be formulated in the form of an ointment, cream, transdermal patch, lotion, gel, shampoo, spray, aerosol, solution, emulsion, or other forms known to one of skill in the art (see e.g., Remington: The Science and Practice of Pharmacy (supra)). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as Freon) or in a squeeze bottle. Moisturizers or humectants can also be added to pharmaceutical compositions and dosage forms if desired. Examples of such additional ingredients are known in the art.

If the antibodies or antigen-binding fragments thereof are administered intranasally, they can be formulated in an aerosol form, spray, mist or in the form of drops. In particular, prophylactic or therapeutic agents can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

In certain embodiments, the antibodies or antigen-binding fragments thereof described herein can be formulated to ensure optimal distribution in vivo. For example, the blood-brain barrier (BBB) excludes many highly hydrophilic compounds. In cases wherein crossing the BBB is desirable, the antibodies or fragments thereof can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhancing targeted drug delivery (see, e.g., Ranade W (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al (1994) J. Biol. Chem. 269:9090); see also K. Keinanen; M. L. Laukkanen (1994) FEBS Lett. 346:123; J. J. Killion; I. J. Fidler (1994) Immunomethods 4:273.

In some embodiments, the multispecific, preferably bispecific, antibody (or antigen-binding fragment thereof) or pharmaceutical composition comprising it is comprised in a kit comprising one or more doses of the multispecific antibody (or antigen-binding fragment thereof) or of the pharmaceutical composition, optionally together with instructions for use. Additionally, the kits may comprise means for administering the multispecific antibody or fragment thereof. The phrase “means for administering” is used to indicate any available implement for systemically administering a drug to a patient, including, but not limited to, a container e.g., pre-filled syringe, a vial and syringe, an injection pen, an auto-injector, an IV drip and bag, an infusion pump, a patch, an infusion bag and needle, etc., which will depend on the administration route, examples of which are provided earlier herein. With such items, a patient may self-administer the drug (i.e., administer the drug without the assistance of a physician) or a medical practitioner may administer the drug. The instructions for use may provide the dose, route of administration, regimen, and total treatment duration for use with the comprised multispecific antibody, fragment thereof, or composition.

### Dosage Regimens

In the methods and uses described herein, a therapeutically or prophylactically effective dose of the multispecific, preferably bispecific, antibody or antigen-fragment thereof is administered to a subject in need thereof (patient), preferably a human. In some embodiments, the dose is effective in treating or preventing a symptom of atopic dermatitis or of a related condition in a human subject. In some embodiments, the dose comprises an amount of the multispecific, preferably bispecific, antibody or antigen-binding fragment thereof effective to achieve a therapeutically or prophylactically effective serum level, preferably throughout the treatment, more preferably throughout the treatment in a human subject.

In particular, the present inventors have found that, in the methods and uses described herein, the multispecific, preferably bispecific, antibody or antigen-binding fragment thereof may be advantageously administered at a dose selected from the group consisting of about 60 mg, about 150 mg, about 300 mg, and about 600 mg, preferably from the group consisting of about 300 mg and about 600 mg.

Accordingly, in some embodiments, there is provided a method of treatment and/or prevention of atopic dermatitis or a related condition in a subject in need thereof, comprising administering a dose of about 60 mg, about 150 mg, about 300 mg, or about 600 mg of the multispecific, preferably bispecific, antibody or antigen-binding fragment thereof described herein to the subject. In some embodiments, a dose of about 60 mg is administered. In some embodiments, a dose of about 150 mg is administered. In preferred embodiments, a dose of about 300 mg is administered. In further preferred embodiments, a dose of about 600 mg is administered.

In some embodiments, there is provided a use of a multispecific, preferably bispecific, antibody or antigen-binding fragment thereof described herein for the manufacture of a medicament for the treatment and/or prevention of atopic dermatitis or a related condition in a subject in need thereof, wherein the multispecific antibody or antigen-binding fragment thereof is administered to the subject at a dose of about 60 mg, about 150 mg, about 300 mg, or about 600 mg. In some embodiments, a dose of about 60 mg is administered. In some embodiments, a dose of about 150 mg is administered. In preferred embodiments, a dose of about 300 mg is administered. In other further preferred embodiments, a dose of about 600 mg is administered.

In some embodiments, there is provided a multispecific, preferably bispecific, antibody, or antigen-binding fragment thereof as described herein for use in the treatment and/or prevention of atopic dermatitis or a related condition in a subject in need thereof, wherein the multispecific antibody or antigen-binding fragment thereof is administered to the subject at a dose of about 60 mg, about 150 mg, about 300 mg, or about 600 mg. In some embodiments, a dose of about 60 mg is administered. In some embodiments, a dose of about 150 mg is administered. In preferred embodiments, a dose of about 300 mg is administered. In other preferred embodiments, a dose of about 600 mg is administered.

In some embodiments, there is provided the use of a multispecific, preferably bispecific, antibody, or antigen-binding fragment thereof as described herein for the treatment and/or prevention of atopic dermatitis or a related condition in a subject in need thereof, wherein the multispecific antibody or antigen-binding fragment thereof is administered to the subject at a dose of about 60 mg, about 150 mg, about 300 mg, or about 600 mg. In some embodiments, a dose of about 60 mg is administered. In some embodiments, a dose of about 150 mg is administered. In preferred embodiments, a dose of about 300 mg is administered. In other preferred embodiments, a dose of about 600 mg is administered.

In some embodiments, the atopic dermatitis is mild atopic dermatitis. In some embodiments, a preferred atopic dermatitis is moderate-to-severe atopic dermatitis.

In some embodiments, the subject, at baseline (prior to treatment), has one or more of the following:
an Eczema Area and Severity Index (EASI) score of ≥12 or ≥16, preferably ≥16;
an Investigator's Global Assessment (IGA) score of ≥3, and/or;
a Body Surface Area (BSA) of ≥10%.

In some embodiments, the subject has a history (e.g., of at least 3, at least 6, at least 9, or at least 12, preferably at least 12, months before the start of the treatment) of inadequate response to atopic dermatitis treatment with a topical medication, or treatment with a topical medication is medically inadvisable for the subject (as per medical professional judgement). “Inadequate response” to treatment with a topical medication is to be determined on a case-by-case basis by the medical professional. As a non-limiting example, a subject exhibiting an inadequate response may fail to achieve and maintain remission or a low disease activity rating (comparable to from IGA 0=clear to 2=mild) despite treatment with an, e.g., daily, regimen of medium or high potency topical medications (e.g., a topical corticosteroid “TCS”) applied for 28 consecutive days, or as recommended by the product prescribing information (whichever is shorter). In some embodiments, the subject is refractory to atopic dermatitis treatment with a topical medication. A treatment may be “medically inadvisable” if, for example, the subject has experienced or is in danger of experiencing significant side effects warranting treatment discontinuation.

In some embodiments, the topical medication is selected from the group consisting of a topical steroid, e.g., corticosteroid, calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist. In some embodiments, the topical corticosteroid is selected from the group consisting of methylprednisolone aceponate, mometasone furoate, fluticasone propionate, betamethasone valerate, triamcinolone, and hydrocortisone butyrate.

The skilled person understands that “about 60 mg” as used herein can be replaced by e.g., from 48 to 72 mg, from 51 to 69 mg, from 54 to 66 mg, from 55 to 65 mg, from 56 to 64 mg, from 57 to 63 mg, from 58 to 62 mg, from 59 to 61 mg, or 60 mg, preferably by 60 mg. Similarly, “about 150 mg” as used herein can be replaced by e.g., from 120 mg to 180 mg, from 128 mg to 173 mg, from 135 mg to 165 mg, from 137 mg to 163 mg, from 138 mg to 162 mg, from 139 mg to 161 mg, from 140 mg to 160 mg, from 141 mg to 159 mg, from 142 mg to 158 mg, from 143 mg to 157 mg, from 144 mg to 156 mg, from 145 mg to 155 mg, from 146 mg to 154 mg, from 147 mg to 153 mg, from 148 mg to 152 mg, from 149 mg to 151 mg, or by 150 mg, preferably by 150 mg. Similarly, “about 300 mg” as used herein can be replaced by e.g., from 240 mg to 360 mg, from 255 mg to 345 mg, from 270 mg to 330 mg, from 273 to 327 mg, from 276 mg to 324 mg, from 279 mg to 321 mg, from 282 mg to 318 mg, from 285 mg to 315 mg, from 288 mg to 312 mg, from 291 mg to 309 mg, from 294 mg to 303 mg, from 296 mg to 302 mg, from 298 mg to 301 mg, or 300 mg, preferably by 300 mg. Similarly, “about 600 mg” as used herein can be replaced by e.g., from 480 mg to 720 mg, from 510 mg to 690 mg, from 540 mg to 660 mg, from 546 mg to 654 mg, from 552 mg to 648 mg, from 558 mg to 642 mg, from 564 mg to 636 mg, from 570 mg to 630 mg, from 576 mg to 624 mg, from 582 mg to 618 mg, from 588 mg to 612 mg, from 594 mg to 606 mg, from 595 mg to 605 mg, from 596 mg to 604 mg, from 597 mg to 603 mg, from 598 mg to 602 mg, from 599 mg to 601 mg, or 600 mg, preferably by 600 mg.

In some embodiments of the methods and uses described herein, the multispecific, preferably bispecific, antibody or antigen-fragment thereof is administered subcutaneously or intravenously, preferably subcutaneously.

In some embodiments, at least one, at least two, at least three, at least four, at least five, at least six, at least seven, at least eight, at least nine, at least ten, at least eleven, at least twelve, at least thirteen, at least fourteen, or at least fifteen doses of the multispecific, preferably bispecific, antibody or antigen-fragment thereof are administered, each dose comprising (“of”) an amount selected from the group consisting of about 60 mg, about 150 mg, about 300 mg, and about 600 mg, preferably from the group consisting of about 300 mg and about 600 mg. In some embodiments, at least one, at least two, at least three, at least four, at least six, or at least seven, for example at least six or at least seven, doses are administered.

The skilled person understands that, in embodiments wherein multiple doses are administered, each dose may comprise the same amount of the multispecific, preferably bispecific, antibody or fragment thereof, or one or more doses may differ. Accordingly, in some embodiments, at least one dose of about 60 mg, for example at least one, at least two, or at least three doses of about 60 mg, and at least one dose, for example at least one, at least two, at least three, or at least four doses of about 150 mg, about 300 mg, or about 600 mg, preferably of about 300 mg or about 600 mg, are administered. In some embodiments, at least one dose of about 150 mg, for example at least one, at least two, or at least three doses of about 150 mg, and at least one dose, for example at least one, at least two, at least three, or at least four doses of about 300 mg or about 600 mg are administered. In some embodiments, at least one dose of about 300 mg, for example at least one, at least two, or at least three doses of about 300 mg, and at least one dose, for example at least one, at least two, at least three, or at least four doses of about 600 mg are administered. In some embodiments, at least one dose of about 600 mg, for example at least one, at least two, or at least three doses of about 600 mg, and at least one dose, for example at least one, at least two, at least three, or at least four doses of about 300 mg are administered. In some embodiments, at least one dose of about 600 mg, for example at least one, at least two, or at least three doses of about 600 mg, and at least one dose, for example at least one, at least two, at least three, or at least four doses of about 150 mg are administered. In some embodiments, at least one dose of about 300 mg, for example at least one, at least two, or at least three doses of about 300 mg, and at least one dose, for example at least one, at least two, at least three, or at least four doses of about 150 mg are administered. In some embodiments, at least one, at least two, at least three, at least four, at least five, at least six, or at least seven doses of about 150 mg are administered. In some embodiments, at least one, at least two, at least three, at least four, at least five, at least six, or at least seven doses of about 300 mg are administered. In some embodiments, at least one, at least two, at least three, at least four, at least five, at least six, or at least seven doses of about 600 mg are administered.

In some embodiments wherein multiple doses are administered, administration may take place at a frequency of once a week (Q1W), once every two weeks (Q2W), once every four weeks (Q4W), once every eight weeks (Q8W), or once every twelve weeks (Q12W). Administration may in some cases take place at a mixed frequency, e.g., doses may be administered at a more frequent regime, e.g., once a week (Q1W), once every two weeks (Q2W), or once every four weeks (Q4W), e.g., for a duration of sixteen weeks of treatment, or of part of sixteen weeks of treatment (e.g., from week 4 to week 16 of treatment), or until a positive response to the treatment is obtained, optionally followed by administration of doses at a less frequent regime, e.g., once every four weeks (Q4W; in the case of a starting frequency of Q1W or Q2W), once every eight weeks (Q8W), or once every twelve weeks (Q12W), e.g., for a duration of at least thirty-six weeks (e.g., thirty-six weeks) of treatment.

In some embodiments, a dosing regimen is administered that is selected from the group consisting of about 60 mg Q1W, about 60 mg Q2W, about 60 mg Q4W, about 60 mg Q8W, about 60 mg Q12W, and a combination thereof. In some embodiments, a dosing regimen is administered that is selected from the group consisting of about 150 mg Q1W, about 150 mg Q2W, about 150 mg Q4W, about 150 mg Q8W, about 150 mg Q12W, and a combination thereof. In some embodiments, a dosing regimen is administered that is selected from the group consisting of about 300 mg Q1W, about 300 mg Q2W, about 300 mg Q4W, about 300 mg Q8W, about 300 mg Q12W, and a combination thereof. In some embodiments, a dosing regimen is administered that is selected from the group consisting of about 600 mg Q1W, about 600 mg Q2W, about 600 mg Q4W, about 600 mg Q8W, about 600 mg Q12W, and a combination thereof.

The timing of dosing is generally measured from the day of the first dose of the multispecific, preferably bispecific, antibody or antigen-binding fragment thereof. However, different healthcare providers may use different naming conventions. Notably, week 1 may be referred to as week 0 by some health care providers, while day 1 may be referred to as day 0 by some health care providers. Thus, as a non-limiting example, it is possible that different physicians will designate, e.g., a dose as being given on week 3 or week 4, while referring to the same dosing schedule (similar considerations would apply to other treatment days/weeks as well).

For consistency, the first day of dosing is referred to herein as day 1. However, it will be understood by a skilled artisan that the naming conventions used herein are used for consistency and should not be construed as limiting, i.e., “Q4W dosing” refers to administration once every four weeks regardless of whether the physician refers to a particular week as “week 3”, “week 4”, etc. It is understood that Q1W, Q2W, Q4W, Q8W, and Q12W administration may in some cases not correspond to administration on exactly the same day of the respective week (compared to the directly preceding or following dose), and may in some cases slightly differ e.g., in +/−1, 2, or 3 days.

In some embodiments, the treatment has a total duration (length) selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks. In some embodiments, the treatment has a total duration of at least sixteen weeks or at least fifty-two weeks, e.g., sixteen weeks or fifty-two weeks. In preferred embodiments, the treatment is maintained as ongoing to maintain control of the atopic dermatitis, related condition, or symptoms thereof, i.e., the treatment has an indefinite duration.

In some embodiments, the treatment may comprise a first treatment period (“TP1”), e.g., having a duration of sixteen weeks (or until a positive response to the treatment is obtained), and a second treatment period (“TP2”), e.g., having a duration of thirty-six weeks or more. The total dose amount and administration frequency between TP1 and TP2 may be the same or may be different, as described earlier herein. In some embodiments, during TP1 at least one, at least two, at least three, at least four, at least five, or at least six doses of the multispecific, preferably bispecific, antibody or antigen-binding fragment thereof are administered. In some embodiments, during TP2, at least one, at least two, at least three, at least four, at least five, at least six, at least seven, at least eight, or at least nine doses of the multispecific, preferably bispecific, antibody or antigen-binding fragment thereof are administered. Preferably, TP2 directly follows TP1, i.e., there is no treatment pause between the two treatment periods. In some embodiments, TP2 is maintained as ongoing; i.e., after TP1 (e.g., having a duration of 16 weeks or until a positive response to the treatment is obtained) the subject is switched to the TP2 dosage regimen to maintain control of the atopic dermatitis, related condition, or symptoms thereof, administered for an indefinite period. In some cases wherein the subject being treated has a positive response to the treatment during TP1, the TP2 dosage regimen may have a decreased administration frequency compared to TP1 (e.g., from Q4W in TP1 to Q8W or Q12W in TP2 etc.). In some cases, e.g., wherein the subject being treated has an inadequate response to the treatment during TP1, the TP2 dosage regimen may maintain the same administration frequency as TP1 (e.g., from Q4W in TP1 to Q4W in TP2 etc.). Administration frequencies are described elsewhere herein.

In some cases wherein the treatment has a fixed duration, the treatment (e.g., of sixteen weeks or fifty-two weeks) may optionally be followed by a safety follow-up period, e.g., of a week, two weeks, four weeks, eight weeks, sixteen weeks, twenty-four weeks, thirty-two weeks, forty weeks, forty-eight weeks, fifty-two weeks, or more, during which no treatment is administered.

Throughout the course of the treatments described herein, as well as during any safety follow-up period where applicable, the subject to which the treatment is being/was administered may be monitored by a physician for the occurrence of adverse effects caused by the treatment. In some embodiments, the methods and uses described herein do not result in any major or severe adverse effects to the subject throughout the course of the treatment, and preferably additionally during any safety follow-up period if applicable. A “major” or “severe” adverse effect, as used herein, refers to the occurrence of a severe side effect, a significant health complication, or any health event caused by the treatment that poses a substantial risk to patient's health. Such an adverse effect typically interrupts usual activities of daily living, significantly affects clinical status, and/or may require intensive therapeutic intervention.

In some embodiments wherein multiple doses are administered, administration may involve a loading phase followed by a maintenance phase. A “loading phase” or “initial phase” is an initial treatment period during which a higher and/or more frequent dose of the multispecific, preferably bispecific, antibody or antigen-binding fragment thereof is typically administered (as compared to the remainder of the treatment period). Without wishing to be bound by theory, embodiments including a loading phase may be advantageous in enabling the antibody or antigen-binding fragment thereof to reach steady-state serum levels quicker relative to dosage regimens not employing a loading phase.

During the “maintenance phase”, the dose and/or administration frequency is typically adjusted to maintain steady-state levels of the antibody. The maintenance phase typically follows (is subsequent to) the loading phase. Administration frequency during the maintenance phase does not necessarily need to be maintained the same throughout its duration. For example, a maintenance phase can involve a certain time period during which the multispecific, preferably bispecific, antibody or antigen-binding fragment thereof is administered at a certain frequency for a period, e.g., from week 4 to week 12 or to week 16 of treatment (an “induction” phase), followed by administration at the same or different frequency (e.g., lower frequency) for another treatment period, the latter period having a definite or indefinite duration as described earlier herein.

A “loading dose” as used herein refers to a dose administered during the loading phase. A “maintenance dose” as used herein refers to a further dose administered after the loading phase. As a non-limiting example, if the last loading dose is administered on week 2 of treatment, and a further dose is administered on week 4 of treatment onwards, then the further dose administered on week 4 can be considered the first maintenance dose.

In some embodiments, a single loading dose is administered. In some embodiments, multiple loading doses are administered, for example at least two or at least three (e.g., three) loading doses. In embodiments wherein multiple loading doses are administered, the loading phase may involve administration of loading doses which is more frequent than the administration of maintenance doses during the maintenance phase.

The amount of multispecific, preferably bispecific, antibody or antigen-binding fragment thereof comprised in the loading doses may be the same as the maintenance doses, or may differ, for example it may be higher or lower. Similarly, the administration frequency of the multispecific, preferably bispecific, antibody or antigen-binding fragment thereof during the loading phase may be the same as the maintenance phase, or may differ, for example it may be more frequent. In embodiments wherein the treatment comprises a first (TP1) and second (TP2) treatment period, TP1 preferably comprises a loading phase. As a non-limiting example, in some embodiments wherein the treatment includes a TP1, TP1 may comprise a loading phase during which at least three loading doses are administered (e.g., on day 1, week 1, and week 2) followed by Q2W or Q4W (e.g., Q4W) administration from week 4 to week 16 (or until a positive response to the treatment is obtained). As another non-limiting example, in some embodiments wherein the treatment includes a TP1 and a TP2, TP1 may comprise a loading phase during which at least three loading doses are administered (e.g., on day 1, week 1, and week 2) followed by Q2W or Q4W (e.g., Q4W) administration from week 4 to week 16 (or until a positive response to the treatment is obtained), and a TP2 thereafter (after week 16) during which administration frequency is Q4W, Q8W, or Q12W.

Accordingly, in some embodiments, there is provided a method of treatment and/or prevention of atopic dermatitis or a related condition in a subject, preferably a human subject, in need thereof, wherein at least one loading dose and at least one maintenance (or further) dose of the multispecific, preferably bispecific, antibody or antigen-binding fragment thereof described herein are administered to the subject.

In some embodiments, there is provided a use of a multispecific, preferably bispecific, antibody or antigen-binding fragment thereof described herein for the manufacture of a medicament for the treatment and/or prevention of atopic dermatitis or a related condition in a subject, preferably a human subject, in need thereof, wherein at least one loading dose and at least one maintenance (or further) dose of the multispecific, preferably bispecific, antibody or antigen-binding fragment thereof described herein are administered to the subject.

In some embodiments, there is provided a multispecific, preferably bispecific, antibody, or antigen-binding fragment thereof as described herein for use in the treatment and/or prevention of atopic dermatitis or a related condition in a subject, preferably a human subject, in need thereof, wherein at least one loading dose and at least one maintenance (or further) dose of the multispecific, preferably bispecific, antibody or antigen-binding fragment thereof described herein are administered to the subject.

In some embodiments, there is provided the use of a multispecific, preferably bispecific, antibody, or antigen-binding fragment thereof as described herein for the treatment and/or prevention of atopic dermatitis or a related condition in a subject, preferably a human subject, in need thereof, wherein at least one loading dose and at least one maintenance (or further) dose of the multispecific, preferably bispecific, antibody or antigen-binding fragment thereof described herein are administered to the subject.

In some embodiments, the at least one loading dose and/or the at least one maintenance dose are administered subcutaneously or intravenously, preferably subcutaneously. In some embodiments, the at least one loading dose and/or the at least one maintenance dose are effective in treating or preventing a symptom of atopic dermatitis or a related condition in a human subject. In some embodiments, the at least one loading dose and/or the at least one maintenance dose comprise an amount of the multispecific, preferably bispecific, antibody or antigen-binding fragment thereof sufficient to achieve a therapeutically effective serum level, preferably throughout the treatment, more preferably through the treatment in a human subject.

The at least one loading dose and/or at least one maintenance dose may comprise any amount of the multispecific, preferably bispecific, antibody or antigen-binding fragment thereof, may be administered at any dosing frequency, and/or may be administered for any treatment duration described earlier herein.

Accordingly, in some embodiments, the at least one loading dose comprises an amount of the multispecific, preferably bispecific, antibody or antigen-binding fragment thereof selected from the group of about 60 mg, about 150 mg, about 300 mg, and about 600 mg, preferably from the group consisting of about 300 mg and about 600 mg. In some embodiments, the at least one loading dose comprises about 60 mg of the antibody or antigen-binding fragment thereof. In some embodiments, the at least one loading dose comprises about 150 mg of the antibody or antigen-binding fragment thereof. In preferred embodiments, the at least one loading dose comprises about 300 mg of the antibody or antigen-binding fragment thereof. In other further preferred embodiments, the at least one loading dose comprises about 600 mg of the antibody or antigen-binding fragment thereof.

In some embodiments, the at least one maintenance dose comprises an amount of the multispecific, preferably bispecific, antibody or antigen-binding fragment thereof selected from the group of about 60 mg, about 150 mg, about 300 mg, and about 600 mg, preferably from the group consisting of about 300 mg and about 600 mg. In some embodiments, the at least one maintenance dose comprises about 60 mg of the antibody or antigen-binding fragment thereof. In some embodiments, the at least one maintenance dose comprises about 150 mg of the antibody or antigen-binding fragment thereof. In preferred embodiments, the at least one maintenance dose comprises about 300 mg of the antibody or antigen-binding fragment thereof. In other further preferred embodiments, the at least one maintenance dose comprises about 600 mg of the antibody or antigen-binding fragment thereof.

The skilled person understands that, the at least one loading dose and the at least one maintenance dose may comprise the same amount of the multispecific, preferably bispecific, antibody or fragment thereof, or they may comprise a different amount. Accordingly, in some embodiments, the at least one loading dose comprises about 60 mg and the at least one maintenance dose comprises about 60 mg of the antibody or antigen-binding fragment thereof. In some embodiments, the at least one loading dose comprises about 60 mg and the at least one maintenance dose comprises about 150 mg of the antibody or antigen-binding fragment thereof. In some embodiments, the at least one loading dose comprises about 60 mg and the at least one maintenance dose comprises about 300 mg of the antibody or antigen-binding fragment thereof. In some embodiments, the at least one loading dose comprises about 60 mg and the at least one maintenance dose comprises about 600 mg of the antibody or antigen-binding fragment thereof. In some embodiments, the at least one loading dose comprises about 150 mg and the at least one maintenance dose comprises about 60 mg of the antibody or antigen-binding fragment thereof. In some embodiments, the at least one loading dose comprises about 150 mg and the at least one maintenance dose comprises about 150 mg of the antibody or antigen-binding fragment thereof. In some embodiments, the at least one loading dose comprises about 150 mg and the at least one maintenance dose comprises about 300 mg of the antibody or antigen-binding fragment thereof. In some embodiments, the at least one loading dose comprises about 150 mg and the at least one maintenance dose comprises about 600 mg of the antibody or antigen-binding fragment thereof. In some embodiments, the at least one loading dose comprises about 300 mg and the at least one maintenance dose comprises about 60 mg of the antibody or antigen-binding fragment thereof. In some embodiments, the at least one loading dose comprises about 300 mg and the at least one maintenance dose comprises about 150 mg of the antibody or antigen-binding fragment thereof. In some embodiments, the at least one loading dose comprises about 300 mg and the at least one maintenance dose comprises about 300 mg of the antibody or antigen-binding fragment thereof. In some embodiments, the at least one loading dose comprises about 300 mg and the at least one maintenance dose comprises about 600 mg of the antibody or antigen-binding fragment thereof. In some embodiments, the at least one loading dose comprises about 600 mg and the at least one maintenance dose comprises about 60 mg of the antibody or antigen-binding fragment thereof. In some embodiments, the at least one loading dose comprises about 600 mg and the at least one maintenance dose comprises about 150 mg of the antibody or antigen-binding fragment thereof. In some embodiments, the at least one loading dose comprises about 600 mg and the at least one maintenance dose comprises about 300 mg of the antibody or antigen-binding fragment thereof. In some embodiments, the at least one loading dose comprises about 600 mg and the at least one maintenance dose comprises about 600 mg of the antibody or antigen-binding fragment thereof.

Treatment duration may be as described earlier herein, and can e.g., be selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks, e.g., it may be at least sixteen weeks or at least fifty-two weeks. In some embodiments, the treatment has an indefinite duration, as discussed earlier herein.

In some embodiments, a loading dose is administered on day 1 of the treatment. In some embodiments, a loading dose is administered on week 1 of the treatment. In some embodiments, a loading dose is administered on week 2 of the treatment. In some embodiments, at least two or at least three loading doses are administered. In some embodiments, a first loading dose is administered on day 1, a second loading dose is administered on week 1, and a third loading dose is administered on week 2 of the treatment, e.g., of the treatment having a duration of at least sixteen weeks, at least fifty-two weeks, or an indefinite duration.

In some embodiments wherein the treatment comprises a first (TP1) treatment period, the first, second, and third loading doses may e.g., be administered, respectively, on day 1, week 1, and week 2 of TP1.

In some embodiments, a TP1 has a duration of sixteen weeks and a preferred TP2 has a duration of at least thirty-six weeks or has an indefinite duration (in such cases the first week of TP2 follows week 16 of the overall treatment). In some embodiments, a TP1 has a duration until a positive response to the treatment is obtained.

In some embodiments, at least two, at least three, at least four, at least five, at least six, at least seven, at least eight, at least nine, at least ten, at least eleven, or at least twelve maintenance doses are administered. In some embodiments, maintenance dose administration frequency is selected from the group consisting of once a week (Q1W), once every two weeks (Q2W), once every four weeks (Q4W), once every eight weeks (Q8W), once every twelve weeks (Q12W), and a combination thereof.

In some embodiments, at least two, at least three, at least four, at least five, at least six, at least seven, at least eight, at least nine, at least ten, at least eleven, or at least twelve maintenance doses are administered, e.g., each dose comprising about 300 mg or 600 mg of the antibody or antigen-binding fragment thereof, with the first maintenance dose administered on week 4 of the treatment, followed by administration once every four weeks (Q4W), once every eight weeks (Q8W), or once every twelve weeks (Q12W).

In some embodiments, at least one, at least two, or at least three loading doses are administered (e.g., three loading doses are administered, for example on day 1, week 1, and week 2, respectively, of the treatment), wherein each loading dose comprises about 60 mg, about 150 mg, about 300 mg, or about 600 mg, preferably about 300 mg or about 600 mg (e.g., about 600 mg) of the antibody or antigen-binding fragment thereof, followed by maintenance dose administration (e.g., starting at week 4 of treatment) at a frequency of Q2W, Q4W, Q8W, or Q12W, preferably Q4W or Q8W (e.g., Q4W), wherein each maintenance dose comprises about 60 mg, about 150 mg, about 300 mg, or about 600 mg, preferably about 300 mg or about 600 mg (e.g., about 600 mg) of the antibody or antigen-binding fragment thereof. In some embodiments, the treatment has a total duration of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks, e.g., sixteen weeks or fifty-two weeks. In some embodiments, the treatment has an indefinite duration, i.e., maintenance dose administration is continued indefinitely.

In some embodiments, at least one, at least two, or at least three (e.g., three, for example on day 1, week 1, and week 2 of treatment, respectively) loading doses comprising about 60 mg of the antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the antibody or antigen-binding fragment thereof are administered. In some embodiments, at least one, at least two, or at least three (e.g., three, for example on day 1, week 1, and week 2 of treatment, respectively) loading doses comprising about 150 mg of the antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the antibody or antigen-binding fragment thereof are administered. In some embodiments, at least one, at least two, or at least three (e.g., three, for example on day 1, week 1, and week 2 of treatment, respectively) loading doses comprising about 300 mg of the antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the antibody or antigen-binding fragment thereof are administered. In some embodiments, at least one, at least two, or at least three (e.g., three, for example on day 1, week 1, and week 2 of treatment, respectively) loading doses comprising about 600 mg of the antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose comprises about 150 mg, about 300 mg, or about 600 mg (e.g., about 300 mg or about 600 mg), and each maintenance dose comprises about 150 mg, about 300 mg, or about 600 mg (e.g., about 300 mg or about 600 mg) of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose comprises about 150 mg and each maintenance dose comprises about 300 mg or about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose comprises about 300 mg and each maintenance dose comprises about 150 mg or about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose comprises about 600 mg and each maintenance dose comprises about 150 mg or about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every four weeks (Q4W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every eight weeks (Q8W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W),
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every eight weeks (Q8W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every four weeks (Q4W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every eight weeks (Q8W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W),
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every eight weeks (Q8W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every four weeks (Q4W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every eight weeks (Q8W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 16 of the treatment (or until a positive response to the treatment is obtained), optionally followed by administration once every twelve weeks (Q12W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W),
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every eight weeks (Q8W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 16 of the treatment (or until a positive response to the treatment is obtained), optionally followed by administration once every twelve weeks (Q12W) thereafter,
   wherein each loading dose and maintenance dose comprises about 600 mg of the multispecific antibody or antigen-binding fragment thereof.

Preferably, each loading and maintenance dose is administered subcutaneously.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every four weeks (Q4W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every eight weeks (Q8W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every eight weeks (Q8W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every twelve weeks (Q12W) thereafter,
   wherein each loading dose and maintenance dose comprises about 150 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every four weeks (Q4W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every eight weeks (Q8W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every eight weeks (Q8W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every twelve weeks (Q12W) thereafter,
   wherein each loading dose and maintenance dose comprises about 300 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 of the treatment (or until a positive response to the treatment is obtained), optionally followed by administration once every four weeks (Q4W), once every eight weeks (Q8W), or once every twelve weeks (Q12W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every four weeks (Q4W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every eight weeks (Q8W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) until week 12 of the treatment (or until a positive response to the treatment is obtained), optionally followed by administration once every twelve weeks (Q12W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every eight weeks (Q8W) thereafter,
   wherein each loading dose and maintenance dose comprises about 600 mg of the multispecific antibody or antigen-binding fragment thereof.

In some embodiments,
a first loading dose is administered on day 1, a second loading dose is administered on week 1, and a third loading dose is administered on week 2 of the treatment;
a maintenance dose is administered on week 4, followed by administration once every four weeks (Q4W) until week 12 of the treatment (or until a positive response to the treatment is obtained), optionally followed by administration once every twelve weeks (Q12W) thereafter,
   wherein each loading dose and maintenance dose comprises about 600 mg of the multispecific antibody or antigen-binding fragment thereof.

In some embodiments, the maintenance dose is administered on week 4, followed by administration once every two weeks (Q2W) until week 16 of the treatment (or until a positive response to the treatment is obtained), optionally followed by administration once every four weeks (Q4W), once every eight weeks (Q8W), or once every twelve weeks (Q12W) thereafter. In some embodiments, the maintenance dose is administered on week 4, followed by administration once every four weeks (Q4W) until week 16 of the treatment (or until a positive response to the treatment is obtained), optionally followed by administration once every four weeks (Q4W), once every eight weeks (Q8W), or once every twelve weeks (Q12W) thereafter.

In some embodiments, the treatment comprises administration of:
i) a first, second, and third loading dose administered on day 1, week 1, and week 2 of the treatment, respectively, and;
ii) a first, second, third, fourth, fifth, sixth, and seventh maintenance dose administered on week 4, week 6, week 8, week 10, week 12, week 14, and week 16 of the treatment, respectively.

In some embodiments, the treatment comprises administration of:
i) a first, second, and third loading dose administered on day 1, week 1, and week 2 of the treatment, respectively, and;
ii) a first, second, third, and fourth, maintenance dose administered on week 4, week 8, week 12, and week 16 of the treatment, respectively.

In some embodiments, the treatment further comprises Q2W, Q4W, Q8W, or Q12W administration thereafter (i.e., after week 12 or week 16). In some embodiments, the loading and maintenance doses comprise an amount of the multispecific, preferably bispecific, antibody or antigen-binding fragment thereof selected from the group consisting of about 60 mg, about 150 mg, about 300 mg, and about 600 mg, preferably from the group consisting of about 300 mg and about 600 mg, as described earlier herein.

In some embodiments, the treatment comprises administration of:
i) a first, second, and third loading dose administered on day 1, week 1, and week 2 of the treatment, respectively, and;
ii) a first, second, and third maintenance dose administered on week 4, week 8, and week 12 of the treatment, respectively.

In some embodiments, the treatment further comprises administration of:
iii) a fourth, fifth, sixth, seventh, eighth, ninth, tenth, eleventh, and twelfth maintenance dose administered on week 16, week 20, week 24, week 28, week 32, week 36, week 40, week 44, and week 48 of the treatment, respectively.

In some embodiments, the treatment further comprises administration of:
iii) a fourth, fifth, sixth, and seventh maintenance dose administered on week 20, week 28, week 36, and week 44 of the treatment, respectively.

In some embodiments, the treatment comprises administration of:
iii) a fourth, fifth, and sixth maintenance dose administered on week 24, week 36, and week 48 of the treatment, respectively.

Each loading and/or maintenance dose may comprise an amount of the multispecific, preferably bispecific, antibody or antigen-binding fragment thereof selected from the group consisting of about 60 mg, about 150 mg, about 300 mg, and about 600 mg, preferably from the group consisting of about 300 mg and about 600 mg (e.g., about 600 mg). Optionally, the treatment may continue utilizing the same administration frequency after week 44 or week 48, as appropriate, for an indefinite period.

In some embodiments, the treatment comprises administration of:
i) a first, second, and third loading dose administered on day 1, week 1, and week 2 of the treatment, respectively, wherein each of the first, second, and third loading dose comprises about 60 mg of the multispecific antibody or antigen-binding fragment thereof, and;
ii) a first, second, and third maintenance dose administered on week 4, week 8, and week 12 of the treatment, respectively, wherein each of the first, second, and third maintenance dose comprises about 60 mg of the multispecific antibody or antigen-binding fragment thereof.

In some embodiments, the treatment further comprises administration of:
iii) a third, fourth, and fifth loading dose administered on week 16, week 17, and week 18 of the treatment, respectively, wherein each of the third, fourth, and fifth loading dose comprises about 600 mg of the multispecific antibody or antigen-binding fragment thereof, and;
iv) a fourth, fifth, sixth, seventh, eighth, ninth, tenth, and eleventh maintenance dose administered on week 20, week 24, week 28, week 32, week 36, week 40, week 44, and week 48 of the treatment, respectively, wherein each of the fourth, fifth, sixth, seventh, eighth, ninth, tenth, and eleventh maintenance dose comprises about 600 mg of the multispecific antibody or antigen-binding fragment thereof.
   Optionally, the treatment may continue utilizing the same administration frequency after week 48 for an indefinite period.

In some embodiments, the treatment comprises administration of:
i) a first, second, and third loading dose administered on day 1, week 1, and week 2 of the treatment, respectively, wherein each of the first, second, and third loading dose comprises about 150 mg of the multispecific antibody or antigen-binding fragment thereof, and;
ii) a first, second, and third maintenance dose administered on week 4, week 8, and week 12 of the treatment, respectively, wherein each of the first, second, and third maintenance dose comprises about 150 mg of the multispecific antibody or antigen-binding fragment thereof.

In some embodiments, the treatment further comprises administration of:
iii) a third, fourth, and fifth loading dose administered on week 16, week 17, and week 18 of the treatment, respectively, wherein each of the third, fourth, and fifth loading dose comprises about 600 mg of the multispecific antibody or antigen-binding fragment thereof, and;
iv) a fourth, fifth, sixth, seventh, eighth, ninth, tenth, and eleventh maintenance dose administered on week 20, week 24, week 28, week 32, week 36, week 40, week 44, and week 48 of the treatment, respectively, wherein each of the fourth, fifth, sixth, seventh, eighth, ninth, tenth, and eleventh maintenance dose comprises about 600 mg of the multispecific antibody or antigen-binding fragment thereof.
   Optionally, the treatment may continue utilizing the same administration frequency after week 48 for an indefinite period.

In some embodiments, the treatment comprises administration of:
i) a first, second, and third loading dose administered on day 1, week 1, and week 2 of the treatment, respectively, wherein each of the first, second, and third loading dose comprises about 300 mg of the multispecific antibody or antigen-binding fragment thereof, and;
ii) a first, second, and third maintenance dose administered on week 4, week 8, and week 12 of the treatment, respectively, wherein each of the first, second, and third maintenance dose comprises about 300 mg of the multispecific antibody or antigen-binding fragment thereof.

In some embodiments, the treatment further comprises administration of:
iii) a fourth, fifth, and sixth maintenance dose administered on week 24, week 36, and week 48 of the treatment, respectively, wherein each of the fourth, fifth, and sixth maintenance dose comprises about 300 mg of the multispecific antibody or antigen-binding fragment thereof.

In some embodiments, the treatment further comprises administration of:
iii) a fourth, fifth, sixth, seventh, eighth, ninth, tenth, eleventh, and twelfth maintenance dose administered on week 16, week 20, week 24, week 28, week 32, week 36, week 40, week 44, and week 48 of the treatment, respectively, wherein each of the fourth, fifth, sixth, seventh, eighth, ninth, tenth, eleventh, and twelfth maintenance dose comprises about 300 mg of the multispecific antibody or antigen-binding fragment thereof.

In some embodiments, the treatment further comprises administration of:
iii) a fourth, fifth, sixth, and seventh maintenance dose administered on week 20, week 28, week 36, and week 44 of the treatment, respectively, wherein each of the fourth, fifth, sixth, and seventh maintenance dose comprises about 300 mg of the multispecific antibody or antigen-binding fragment thereof.
   Optionally, the treatment may continue utilizing the same administration frequency after week 44 or week 48, as appropriate, for an indefinite period.

In some embodiments, the treatment comprises administration of:
i) a first, second, and third loading dose administered on day 1, week 1, and week 2 of the treatment, respectively, wherein each of the first, second, and third loading dose comprises about 300 mg of the multispecific antibody or antigen-binding fragment thereof, and;
ii) a first, second, third, and fourth maintenance dose administered on week 4, week 8, week 12, and week 16 of the treatment, respectively, wherein each of the first, second, third, and fourth maintenance dose comprises about 300 mg of the multispecific antibody or antigen-binding fragment thereof.

In some embodiments, the treatment further comprises administration of:
iii) a fifth and sixth maintenance dose administered on week 28 and week 40 of the treatment, respectively, wherein each of the fifth and sixth maintenance dose comprises about 300 mg of the multispecific antibody or antigen-binding fragment thereof.

In some embodiments, the treatment further comprises administration of:
iii) a fifth, sixth, seventh, eighth, ninth, tenth, eleventh, and twelfth maintenance dose administered on week 20, week 24, week 28, week 32, week 36, week 40, week 44, and week 48 of the treatment, respectively, wherein each of the fifth, sixth, seventh, eighth, ninth, tenth, eleventh, and twelfth maintenance dose comprises about 300 mg of the multispecific antibody or antigen-binding fragment thereof.

In some embodiments, the treatment further comprises administration of:
iii) a fifth, sixth, seventh, and eighth maintenance dose administered on week 24, week 32, week 40, and week 48 of the treatment, respectively, wherein each of the fifth, sixth, seventh, and eighth maintenance dose comprises about 300 mg of the multispecific antibody or antigen-binding fragment thereof.
   Optionally, the treatment may continue utilizing the same administration frequency after week 40 or week 48, as appropriate, for an indefinite period.

In some embodiments, the treatment comprises administration of:
i) a first, second, and third loading dose administered on day 1, week 1, and week 2 of the treatment, respectively, wherein each of the first, second, and third loading dose comprises about 600 mg of the multispecific antibody or antigen-binding fragment thereof, and;
ii) a first, second, and third maintenance dose administered on week 4, week 8, and week 12 of the treatment, respectively, wherein each of the first, second, and third maintenance dose comprises about 600 mg of the multispecific antibody or antigen-binding fragment thereof.

In some embodiments, the treatment further comprises administration of:
iii) a fourth, fifth, and sixth maintenance dose administered on week 24, week 36, and week 48 of the treatment, respectively, wherein each of the fourth, fifth, and sixth maintenance dose comprises about 600 mg of the multispecific antibody or antigen-binding fragment thereof.

In some embodiments, the treatment further comprises administration of:
iii) a fourth, fifth, sixth, seventh, eighth, ninth, tenth, eleventh, and twelfth maintenance dose administered on week 16, week 20, week 24, week 28, week 32, week 36, week 40, week 44, and week 48 of the treatment, respectively, wherein each of the fourth, fifth, sixth, seventh, eighth, ninth, tenth, eleventh, and twelfth maintenance dose comprises about 600 mg of the multispecific antibody or antigen-binding fragment thereof.

In some embodiments, the treatment further comprises administration of:
iii) a fourth, fifth, sixth, and seventh maintenance dose administered on week 20, week 28, week 36, and week 44 of the treatment, respectively, wherein each of the fourth, fifth, sixth, and seventh maintenance dose comprises about 600 mg of the multispecific antibody or antigen-binding fragment thereof.

Optionally, the treatment may continue utilizing the same administration frequency after week 44 or week 48, as appropriate, for an indefinite period.

In some embodiments, the treatment comprises administration of:
i) a first, second, and third loading dose administered on day 1, week 1, and week 2 of the treatment, respectively, wherein each of the first, second, and third loading dose comprises about 600 mg of the multispecific antibody or antigen-binding fragment thereof, and;
ii) a first, second, third, and fourth maintenance dose administered on week 4, week 8, week 12, and week 16 of the treatment, respectively, wherein each of the first, second, third, and fourth maintenance dose comprises about 600 mg of the multispecific antibody or antigen-binding fragment thereof.

In some embodiments, the treatment further comprises administration of:
iii) a fifth and sixth maintenance dose administered on week 28 and week 40 of the treatment, respectively, wherein each of the fifth and sixth maintenance dose comprises about 600 mg of the multispecific antibody or antigen-binding fragment thereof.

In some embodiments, the treatment further comprises administration of:
iii) a fifth, sixth, seventh, eighth, ninth, tenth, eleventh, and twelfth maintenance dose administered on week 20, week 24, week 28, week 32, week 36, week 40, week 44, and week 48 of the treatment, respectively, wherein each of the fifth, sixth, seventh, eighth, ninth, tenth, eleventh, and twelfth maintenance dose comprises about 600 mg of the multispecific antibody or antigen-binding fragment thereof.

In some embodiments, the treatment further comprises administration of:
iii) a fifth, sixth, seventh, and eighth maintenance dose administered on week 24, week 32, week 40, and week 48 of the treatment, respectively, wherein each of the fifth, sixth, seventh, and optionally eighth maintenance dose comprises about 600 mg of the multispecific antibody or antigen-binding fragment thereof.

Optionally, the treatment may continue utilizing the same administration frequency after week 40 or week 48, as appropriate, for an indefinite period.

It is understood that, in the embodiments described above involving maintenance dose administration up to week 40, week 44, week 48, or week 52 of the treatment, administration of maintenance doses may continue past those weeks following the same frequency, for example Q2W, Q4W, Q8W, or Q12W, as applicable, for an indefinite period, i.e., administration of maintenance dosage may be maintained as an ongoing treatment.

In this disclosure, maintenance doses administered from week 4 to week 16 of the treatment may also alternatively be referred to as “induction” doses.

In an aspect, the treatments described herein comprise administration of the multispecific, preferably bispecific, antibody or antigen-binding fragment thereof to a subject in need thereof, wherein the subject has previously received at least one dose (e.g., at least one, at least two, or at least three doses) of the antibody or antigen-binding fragment thereof. In an aspect, the treatments described herein comprise administration of the multispecific, preferably bispecific, antibody or antigen-binding fragment thereof to a subject in need thereof, wherein the subject has received at least one loading dose (e.g., at least one, at least two, or at least three loading doses, e.g., three loading doses) of the antibody or antigen-binding fragment thereof. In some embodiments, at least one maintenance dose is administered to the subject, as described earlier herein. Non-limiting examples of maintenance dose administration of the multispecific antibody or antigen-binding fragment thereof are subcutaneous administration of about 60 mg Q2W, about 60 mg Q4W, about 60 mg Q8W, or about 60 mg Q12W. Additional non-limiting examples of maintenance dose administration of the multispecific antibody or antigen-binding fragment thereof are subcutaneous administration of about 150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, or about 600 mg Q12W, among which about 300 mg Q2W, about 300 mg Q4W, about 300 mg Q8W, about 300 mg Q12W, about 600 mg Q2W, about 600 mg Q4W, about 600 mg Q8W, or about 600 mg Q12W (e.g., about 300 mg Q4W, about 600 mg Q4W) are preferred.

In some embodiments, a maintenance dose is administered two weeks, four weeks, eight weeks, or twelve weeks after the last dose (e.g., loading dose) has been administered to the subject. In some embodiments, a maintenance dose is administered one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen weeks, nineteen weeks, twenty weeks, twenty-one weeks, twenty-two weeks, twenty-three weeks, twenty-four weeks, twenty-five weeks, twenty-six weeks, twenty-seven weeks, twenty-eight weeks, twenty-nine weeks, thirty weeks, thirty-one weeks, thirty-two weeks, thirty-three weeks, thirty-four weeks, thirty-five weeks, thirty-six weeks, thirty-seven weeks, thirty-eight weeks, thirty-nine weeks, forty weeks, forty-one weeks, forty-two weeks, forty-three weeks, forty-four weeks, forty-five weeks, forty-six weeks, forty-seven weeks, forty-eight weeks, forty-nine weeks, fifty weeks, fifty-one weeks, or fifty-two weeks after the last dose (e.g., loading dose) has been administered to the subject.

The subject being administered a treatment described herein may experience an improvement (positive response) in an atopic dermatitis-associated parameter. Such a subject may alternatively be called a responder to the treatment. Non-limiting examples of atopic dermatitis-associated parameters are Investigator's Global Assessment (IGA) score (e.g., vIGA-AD™ (Validated Investigator Global Assessment scale for Atopic Dermatitis)), Eczema Area and Severity Index (EASI) score (e.g., described in Hanifin et al. 2022, Dermatitis 33 (3): 187-92), Dermatology Life Quality Index (DLQI) score (e.g., as described in Hongbo et al. 2005, J Invest Dermatol 125 (4): 659-64), Peak Pruritus Numeric Rating scale (PP-NRS) score (e.g., as described in Yosipovitch et al. 2019, Br J Dermatol 181 (4): 761-9), and Body Surface Area (BSA) score, discussed earlier herein. The skilled person is aware of how to measure these parameters, and exemplary calculations are provided in the Examples section later herein.

Accordingly, in some embodiments, the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI) score;
Peak Pruritus Numeric Rating scale (PP-NRS) score, and;
a combination thereof.

In some embodiments, the improvement is selected from the group consisting of:
a decrease in Investigator's Global Assessment (IGA) score by at least 2 points, in particular a decrease in IGA score by at least 1 or by at least 2 points, preferably by at least 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment, for example at or before week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, or week 52 of treatment.

In some embodiments, a subject that is a responder to the treatment achieves (relative to baseline) a decrease in Investigator's Global Assessment (IGA) score by at least 2 points, and preferably further achieves clear or almost clear status, e.g., at or before week 16 or week 52 of the treatment.

The methods and uses described herein may result in an improvement (relative to baseline) in an atopic dermatitis-associated biomarker (positive response) in the subject to which the multispecific, preferably bispecific, antibody or antigen-binding fragment thereof is administered, for example a decrease in the concentration (level) of the biomarker in the subject's tissues and/or bodily fluids (e.g., blood, serum, plasma, and the like).

In some embodiments, the methods and uses described herein comprise determining the level of an atopic dermatitis-associated biomarker in a subject, selecting a subject with an elevated level of the atopic dermatitis-associated biomarker, and administering the multispecific, preferably bispecific, antibody or antigen-binding fragment thereof to the subject, as described herein.

In some embodiments, the atopic dermatitis-associated biomarker is selected from the group consisting of CCL17 (TARC), pSTAT6, IFNγ, IL-13 (bioactive), IL-18 (bioactive) (e.g., serum free IL-18), and IL-18BP (e.g., serum IL-18BP), preferably from the group consisting of CCL17 (TARC), pSTAT6, IFNγ, IL-13 (bioactive), and IL-18 (bioactive) (e.g., serum free IL-18).

In some embodiments, the one or more AD-associated biomarkers are selected from the group consisting of CCL17 (TARC), pSTAT6, IFNγ, IL-13 (bioactive), IL-18 (bioactive) (e.g., serum free IL-18), IL-18BP (e.g., serum IL-18BP), IgE (e.g., serum IgE), CCL26 (eotaxin-3), CCL22 (MDC2), hsCRP, CD40, IL-24, and IL-22. The skilled person is aware of assays that can be used to measure atopic-dermatitis biomarkers, for example using ELISA, flow cytometry, or any other suitable assay, examples of which are provided in the experimental section later herein.

In some embodiments, the improvement (relative to baseline) in the atopic dermatitis-associated biomarker, e.g., the decrease in its levels in the subject (e.g., in its tissues, serum or blood), is at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, or more. In some embodiments, the improvement (relative to baseline) in the atopic dermatitis-associated biomarker, e.g., the decrease in its levels in the subject, is obtained at or before week 16 or week 52 of the treatment, for example at or before week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, or week 52 of treatment.

In some embodiments, the methods and uses described herein result in the alleviation (decrease) of a symptom associated with atopic dermatitis relative to baseline (positive response), preferably selected from the group consisting of erythema, induration/papulation, excoriations, lichenification, pruritus (itch), dry skin, thickened skin, cracked skin, scaly skin, raw skin, skin sensitivity, swollen skin, scaly and dry eczematous lesions, and a combination thereof. In some embodiments, the alleviation is at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, or more, relative to baseline. In some embodiments, the alleviation is obtained at or before week 16 or week 52 of the treatment, for example at or before week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, or week 52 of treatment. Alleviation as used herein may also refer to the complete disappearance (absence) of symptoms following administration of a treatment described herein.

In some embodiments, the symptom is erythema, and the treatment results in a decrease of at least 1 point or at least 2 points in severity (e.g., from 3=severe to 2=moderate, 1=mild, or 0=absent) based on the Eczema Area and Severity Index (EASI) score. A score of 3 refers to erythema being deep/dark red, a score of 2 to dull red, clearly distinguishable, and a score of 1 to faintly detectable, very light pink. In some embodiments, the erythema is located in the head & neck area, the trunk, the upper limbs, and/or the lower limbs area of the subject.

In some embodiments, the symptom is induration/papulation, and the treatment results in a decrease of at least 1 point or at least 2 points in severity (e.g., from 3=severe to 2=moderate, 1=mild, or 0=absent) based on the Eczema Area and Severity Index (EASI) score. A score of 3 refers to induration/papulation having marked and extensive elevation, a score of 2 to clearly perceptible elevation but not extensive, and a score of 1 to barely perceptible elevation. In some embodiments, the induration/papulation is located in the head & neck area, the trunk, the upper limbs, and/or the lower limbs area of the subject.

In some embodiments, the symptom is excoriations, and the treatment results in a decrease of at least 1 point or at least 2 points in severity (e.g., from 3=severe to 2=moderate, 1=mild, or 0=absent) based on the Eczema Area and Severity Index (EASI) score. A score of 3 refers to the presence of many erosive or crusty lesions, a score of 2 to several linear marks of skin with some showing evidence of deeper skin damage (erosion, crust), and a score of 1 to scant evidence of excoriations with no signs of deeper skin damage (erosion, crust). In some embodiments, the excoriations are located in the head & neck area, the trunk, the upper limbs, and/or the lower limbs area of the subject.

In some embodiments, the symptom is lichenification, and the treatment results in a decrease of at least 1 point or at least 2 points in severity (e.g., from 3=severe to 2=moderate, 1=mild, or 0=absent) based on the Eczema Area and Severity Index (EASI) score. A score of 3 refers to the presence of thickened indurated skin with skin markings visibly portraying an exaggerated crisscross pattern, a score of 2 to definite thickening of the skin with skin markings exaggerated so that they form a visible crisscross pattern, and a score of 1 to slight thickening of the skin discernible only by touch and with skin marking minimally exaggerated. In some embodiments, the lichenification is located in the head & neck area, the trunk, the upper limbs, and/or the lower limbs area of the subject.

In some embodiments, the related condition to atopic dermatitis is selected from the group consisting of conditions related to inflammation, such as allergy (e.g., skin allergy, allergy to certain foods, pollen, mold, dust mite, animals, etc.), asthma (such as allergic and non-allergic asthma), infectious disorders, skin infection, cutaneous infection, eczema herpeticum, erysipelas, cellulitis, extra-cutaneous infection, encephalitis, endocarditis, infectious arthropathy, enterocolitis, septicemia, respiratory infection, upper respiratory tract infection, lower respiratory tract infection, lung infection, cardiac infection, brain infection, bone infection, and gastrointestinal infection, increased colonization with Staphylococcus aureus in lesional and non-lesional skin, autoimmune disorders, respiratory disorders, neuropsychiatric disorders, musculoskeletal disorders, and cardiovascular disorders associated with atopic dermatitis.

In some embodiments, the methods and uses described herein decrease the dependence of the subject to which treatment is administered on treatment with a topical medication, e.g., the topical medications described earlier herein. In some embodiments, the subject has moderate-to-severe AD. In some embodiments, administration of a treatment described herein decreases the required dosage of a topical medication, e.g., of a TCS, by at least 5%, at least 10%, at least 15%, at least 20%, at least 30%, at least 40%, at least 50% or more, as compared to the dosage required in the absence of treatment with the multispecific antibody or antigen-binding fragment thereof.

In some embodiments of the methods and uses described herein, the treatment comprises administration of at least one additional therapeutic agent (other than the multispecific antibody or antigen-binding fragment thereof). In some embodiments, the at least one additional agent is administered concurrently or sequentially (before or after) with the multispecific antibody or antigen-binding fragment thereof. It is understood that concurrent administration does not only refer to administration at exactly the same time, and also includes cases wherein the antibody or antigen-binding fragment thereof and the additional therapeutic agent are exerting their effects simultaneously. The multispecific antibody or antigen-binding fragment thereof and the additional therapeutic agent may be comprised in the same or different pharmaceutical compositions, which will depend on the nature of the additional therapeutic agent.

In some embodiments, the at least one additional therapeutic agent is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

In some embodiments of the methods and uses described herein, the treatment further comprises administration of a moisturizer to the subject. In some embodiments, the multispecific, preferably bispecific, antibody or antigen-binding fragment thereof is administered subcutaneously, as described earlier herein, and the moisturizer is administered topically, for example to skin affected by atopic dermatitis or a related condition. It is understood that in such embodiments, the antibody or antigen-binding fragment thereof and the moisturizer are preferably comprised in different pharmaceutical compositions. Suitable moisturizers include lotions (e.g., steroid-based lotions), creams, ointments, oil-based moisturizers, and emollients. In some embodiments, the administration of the moisturizer has a different frequency from the administration of the antibody or antigen-binding fragment thereof. In some embodiments, application of the moisturizer is at least daily or at least twice daily, throughout the course of the treatment.

### Manufacture of Multispecific Antibody and Antigen-Binding Fragments Thereof

The multispecific, preferably bispecific, antibodies and antigen-binding fragments thereof described herein can be generated using a number of techniques available to the skilled person. As a non-limiting example, the antibodies and antigen-binding fragments thereof can be generated as described in WO2023/209568 or WO2018/057955. As another non-limiting example, the antibodies and antigen-binding fragments thereof can be generated recombinantly, by introducing nucleic acid molecules encoding them into expression vectors and expressing them in host cells. In some embodiments, the first part that binds to IL-18 and the second part that binds to IL-13 are encoded by the same nucleic acid molecule or expression vector. In some embodiments, the first part that binds to IL-18 and the second part that binds to IL-13 are encoded by separate nucleic acid molecules or expression vectors. The nucleic acid molecules can be produced using a variety of techniques, for example by de novo solid-phase DNA synthesis, amplification and/or mutagenesis of any existing molecule using PCR, and the like.

Suitable expression vectors are discussed earlier herein. The skilled person understands that the choice of the expression vector will depend on the intended host cell for expression. Typically, an expression vector additionally comprises a promoter and other regulatory sequences, operably linked to the nucleic acid molecule encoding the first part that binds to IL-18 and/or the second part that binds to IL-13. In some embodiments, the promoter is a constitutive or an inducible promoter. The expression vector may additionally comprise a nucleotide sequence encoding for a secretion signal, operably linked to the first part that binds to IL-18 and/or the second part that binds to IL-13. The antibodies or antigen-binding fragments thereof are typically recovered from cell culture medium as secreted polypeptides, but may also be recovered from host cell lysate when produced without secretion signals. If the antibodies or antigen-binding fragments thereof are membrane-bound, they can be released from the membrane using e.g., a suitable detergent solution (e.g., Triton-X 100).

The host cell can be, but is not limited to, a prokaryotic cell (e.g., a bacterium, for example E. coli), a eukaryotic cell (e.g., a mammalian cell), an insect cell, or a human cell. The host cell may correspond to a cell line. Suitable eukaryotic cell lines include, but are not limited to, Vero cells, HeLa cells, COS cells, CHO cells, HEK cells, BHK cells, and MDCKII cells. Other examples of suitable eukaryotic cell lines include, but are not limited to, COS-1 (ATCC No. CRL 1650), COS-7 (ATCC No. CRL 1651), BHK570 (ATCC No. CRL 10314), DHFR-CHO (see Urlaub et al., 1986, Somatic Cell Mol. Genet. 12, 555-556), mouse Sertoli cells, monkey kidney cells, African green monkey kidney cells (ATCC No. CRL 1587), canine kidney cells (ATCC CCL34), human lung cells (ATCC CCL 75), Hep G2 and myeloma or lymphoma cells (e.g., NSO; see U.S. Pat. No. 5,807,715), Sp2/0, YO cells. Suitable insect cells, include, but are not limited to, Sf9 cells. In some embodiments, the host cell is a HEK cell or a CHO cell.

Methods for introducing expression vectors comprising the nucleic acid molecules of interest can vary depending on the type of host cell. For example, calcium chloride transfection is commonly utilized for prokaryotic cells, whereas calcium phosphate treatment or electroporation may be used for other cellular hosts (see generally Sambrook, et al., Molecular Cloning: A Laboratory Manual (4^{th} Edition), 2012, Cold Spring Harbor Laboratory Press, USA). Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22 (e.g., see Elliot and O'Hare, (1997) Cell 88:223), agent-enhanced uptake of DNA, and ex vivo transduction.

Multispecific, preferably bispecific, antibodies and antigen-binding fragments thereof may be produced via co-expression of the first part- and second-part described herein using a single host cell. In some embodiments wherein the antibody or antigen-binding fragment thereof comprises a first light chain of the lambda type and a second light chain of the kappa type, co-expression in a single host cell may be particularly advantageous as chain mispairing can be minimized. Alternatively, multispecific, e.g., bispecific, antibodies may be produced using multiple host cells, e.g., a first host cell may express the first part that binds to IL-18 and a second host cell may express the second part that binds to IL-13. In such methods, each host cell may comprise a nucleic acid molecule or expression vector encoding the first- and/or second-part. The host cells may be cultured in the same or different cultures. Production of the multispecific, preferably bispecific, antibody or antigen-binding fragment thereof will generally involve culturing the host cell(s) under conditions sufficient (conducive) to express the first- and second-part followed by recovery of the first- and second-part. In some cases, the produced polypeptides are recovered and optionally purified from recombinant cell proteins or polypeptides to obtain preparations that are substantially homogeneous as to heteromultimer. As a first step, the culture medium or lysate is typically centrifuged to remove particulate cell debris. Alternatively, depth filtration followed by sterile filtration may be used. The produced antibodies or antigen-binding fragments thereof (or the first part and/or second part) can be conveniently purified e,g., by hydroxylapatite chromatography, gel electrophoresis, dialysis, or affinity chromatography (e.g., on a CaptureSelect CH1-XL resin (Thermofisher Scientific, WA, MA, USA)). Other techniques for protein purification such as fractionation on an ion-exchange column, ethanol precipitation, reverse phase HPLC, chromatography on silica, chromatography on heparin sepharose, chromatography on an anion or cation exchange resin (such as a polyaspartic acid column), chromatofocusing, SDS-PAGE, protein A-affinity chromatography, and ammonium sulfate precipitation are also available. Purity may be e.g., determined using size exclusion chromatography, e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA).

### ADDITIONAL ASPECTS AND EMBODIMENTS

In an aspect, there is provided a multispecific antibody or antigen-binding fragment thereof, wherein the antibody comprises:
i) a first part comprising means for binding to IL-18, and;
ii) a second part comprising means for binding to IL-13, for use in the treatment and/or prevention of atopic dermatitis in a subject in need thereof, wherein at least one loading dose and at least one maintenance dose are administered to the subject.

In a further aspect, there is provided a method of treatment and/or prevention of atopic dermatitis in a subject in need thereof, comprising administering at least one loading dose and at least one maintenance dose of a multispecific antibody or antigen-binding fragment thereof to the subject, wherein the antibody comprises:
i) a first part comprising means for binding to IL-18, and;
ii) a second part comprising means for binding to IL-13.

In a further aspect, there is provided a use of a multispecific antibody or antigen-binding fragment thereof for the manufacture of a medicament for the treatment and/or prevention of atopic dermatitis in a subject in need thereof, wherein at least one loading dose and at least one maintenance dose are administered to the subject, and wherein the multispecific antibody comprises:
i) a first part comprising means for binding to IL-18, and;
ii) a second part comprising means for binding to IL-13.

In a further aspect, there is provided a use of a multispecific antibody or antigen-binding fragment thereof for the treatment and/or prevention of atopic dermatitis in a subject in need thereof, wherein at least one loading dose and at least one maintenance dose are administered to the subject, and wherein the multispecific antibody comprises:
i) a first part comprising means for binding to IL-18, and;
ii) a second part comprising means for binding to IL-13.

In a further aspect, there is provided a method of treatment and/or prevention of atopic dermatitis in a subject in need thereof, comprising administering at least one loading dose and at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the manufacture of a medicament for the treatment and/or prevention of atopic dermatitis in a subject in need thereof, wherein at least one loading dose and at least one maintenance dose are administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the treatment and/or prevention of atopic dermatitis in a subject in need thereof, wherein at least one loading dose and at least one maintenance dose are administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In preferred embodiments, the multispecific antibody is bispecific.

In some embodiments, the multispecific antibody is an IL-18 and IL-13 antagonist.

In some embodiments, the means for binding to IL-18 or the first part that binds to IL-18 comprises a first variable heavy region (VH1) comprising or consisting of SEQ ID NO: 41 and a first variable light region (VL1) comprising or consisting of SEQ ID NO: 13, and the means for binding to IL-13 or the second part that binds to IL-13 comprises a second variable heavy region (VH2) comprising or consisting of SEQ ID NO: 55 and a second variable light region (VL2) comprising or consisting of SEQ ID NO: 27.

In some embodiments, the first part that binds to IL-18, or that comprises means for binding to IL-18, comprises a first light chain (LC1) that is a lambda light chain and the second part that binds to IL-13, or that comprises means for binding to IL-13, comprises a second light chain (LC2) that is a kappa light chain.

In some embodiments, the first part that binds to IL-18, or that comprises means for binding to IL-18, comprises a first light chain (LC1) comprising or consisting of SEQ ID NO: 14 and the second part that binds to IL-13, or that comprises means for binding to IL-13, comprises a second light chain (LC2) comprising or consisting of SEQ ID NO: 28.

In some embodiments, the first part that binds to IL-18, or that comprises means for binding to IL-18, comprises a first Fc polypeptide, the second part that binds to IL-13, or that comprises means for binding to IL-13, comprises a second Fc polypeptide, and the first and/or second Fc polypeptide comprises the amino acid sequence SEQ ID NO: 59 or a variant thereof.

In some embodiments, the first and/or second Fc polypeptide comprises a variant of SEQ ID NO: 59 comprising one or more amino acid substitutions selected from the group consisting of one or more half-life extension substitutions, e.g., an M252Y, an S254T, and a T256E substitution (“YTE”), and one or more K-i-H substitutions, e.g., an S354C and/or a T366W substitution or a Y349C, a T366S, an L368A, and/or a Y407V substitution, according to EU numbering.

In some embodiments, the first part that binds to IL-18, or that comprises means for binding to IL-18, comprises a first Fc polypeptide comprising a variant of SEQ ID NO: 59 comprising: an M252Y, an S254T, and a T256E substitution (“YTE”), and a Y349C, a T366S, an L368A, and a Y407V substitution, and the second part that binds to IL-13, or that comprises means for binding to IL-13, comprises a second Fc polypeptide comprising a variant of SEQ ID NO: 59 comprising: an M252Y, an S254T, and a T256E substitution (“YTE”), and an S354C and a T366W substitution, according to EU numbering.

In some embodiments, the first part that binds to IL-18, or that comprises means for binding to IL-18, comprises a first Fc polypeptide comprising a variant of SEQ ID NO: 59 comprising: an M252Y, an S254T, and a T256E substitution (“YTE”), and an S354C and a T366W substitution, and the second part that binds to IL-13, or that comprises means for binding to IL-13, comprises a second Fc polypeptide comprising a variant of SEQ ID NO: 59 comprising: an M252Y, an S254T, and a T256E substitution (“YTE”), and a Y349C, a T366S, an L368A, and a Y407V substitution, according to EU numbering.

In some embodiments, the first part that binds to IL-18, or that comprises means for binding to IL-18, comprises a first heavy chain (HC1) comprising or consisting of SEQ ID NO: 42 and a first light chain (LC1) comprising or consisting of SEQ ID NO: 14, and the second part that binds to IL-13, or that comprises means for binding to IL-13, comprises a second heavy chain (HC2) comprising or consisting of SEQ ID NO: 56 and a second light chain (LC2) comprising or consisting of SEQ ID NO: 28.

In some embodiments, the first part that binds to IL-18, or that comprises means for binding to IL-18, comprises a first heavy chain (HC1) comprising or consisting of SEQ ID NO: 57 and a first light chain (LC1) comprising or consisting of SEQ ID NO: 14, and the second part that binds to IL-13, or that comprises means for binding to IL-13, comprises a second heavy chain (HC2) comprising or consisting of SEQ ID NO: 58 and a second light chain (LC2) comprising or consisting of SEQ ID NO: 28.

In preferred embodiments, the IL-18 and the IL-13 are human. In preferred embodiments, the subject is a human.

In preferred embodiments, the atopic dermatitis is moderate-to-severe atopic dermatitis.

In some embodiments, the at least one loading dose and/or the at least one maintenance dose are administered subcutaneously or intravenously, preferably subcutaneously.

In some embodiments, the at least one loading dose and/or the at least one maintenance dose comprise an amount of the multispecific antibody or antigen-binding fragment thereof sufficient to achieve a therapeutically effective serum level. In some embodiments, the therapeutically effective serum level is maintained throughout the course of the treatment.

In some embodiments, the at least one loading dose comprises an amount of the multispecific antibody or antigen-binding fragment thereof selected from the group consisting of about 60 mg, about 150 mg, about 300 mg, and about 600 mg. In some embodiments, the at least one maintenance dose comprises an amount of the multispecific antibody or antigen-binding fragment thereof selected from the group consisting of about 60 mg, about 150 mg, about 300 mg, and about 600 mg.

In some embodiments, at least two or at least three loading doses are administered. In some embodiments, a first loading dose is administered on day 1, a second loading dose is administered on week 1, and a third loading dose is administered on week 2 of the treatment.

In some embodiments, at least two, at least three, at least four, at least five, at least six, at least seven, at least eight, at least nine, at least ten, at least eleven, or at least twelve maintenance doses are administered.

In some embodiments, maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

In some embodiments, at least two maintenance doses are administered, and the first maintenance dose is administered on week 4 of the treatment, followed by administration once every two weeks (Q2W), once every four weeks (Q4W), once every eight weeks (Q8W), or once every twelve weeks (Q12W).

In some embodiments, the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

In some embodiments, at least one, at least two, or at least three (e.g., three) loading doses comprising about 60 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the multispecific antibody or antigen-binding fragment thereof are administered.

In some embodiments, at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 150 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 300 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered.

In some embodiments,
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter, wherein each loading dose and maintenance dose comprises about 600 mg of the multispecific antibody or antigen-binding fragment thereof. In some embodiments, a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter. In some embodiments, a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

In some embodiments, the subject, at baseline, has one or more of the following:
an Eczema Area and Severity Index (EASI) score of ≥12 or ≥16, preferably ≥16,
an Investigator's Global Assessment (IGA) score of ≥3, and/or
a Body Surface Area (BSA) of ≥10%.

In some embodiments, the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI);
Peak Pruritus Numeric Rating scale (PP-NRS) score, and;
a combination thereof.

In some embodiments, the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof, optionally obtained at or before week 16 or week 52 of the treatment.

In some embodiments, the treatment results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP).

In some embodiments, the subject has a history of inadequate response to treatment with a topical medication or is a subject for whom topical medications are medically inadvisable.

In some embodiments, the topical medication is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist.

In some embodiments, the topical corticosteroid is selected from the group consisting of methylprednisolone aceponate, mometasone furoate, fluticasone propionate, betamethasone valerate, triamcinolone, and hydrocortisone butyrate.

In some embodiments, the treatment results in alleviation of a symptom associated with atopic dermatitis relative to baseline, preferably selected from the group consisting of erythema, induration/papulation, excoriations, lichenification, pruritus, dry skin, thickened skin, cracked skin, scaly skin, raw skin, skin sensitivity, swollen skin, scaly and dry eczematous lesions, and a combination thereof.

In some embodiments, the treatment comprises administration of at least one additional therapeutic agent. In some embodiments, the at least one additional therapeutic agent is administered concurrently or sequentially with the multispecific antibody or antigen-binding fragment thereof.

In some embodiments, the additional therapeutic agent is selected from the group consisting of a topical steroid, e.g., corticosteroid, a calcineurin inhibitor, e.g., topical calcineurin inhibitor, tacrolimus, pimecrolimus a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

In some embodiments, the multispecific antibody or antigen-binding fragment thereof is comprised in a pharmaceutical composition, and the pharmaceutical composition optionally comprises one or more pharmaceutically acceptable ingredients. In some embodiments, the pharmaceutical composition is comprised in a kit comprising one or more doses of the pharmaceutical composition, optionally together with instructions for use.

In a further aspect, there is provided a method of treatment and/or prevention of atopic dermatitis in a subject in need thereof, wherein the subject has previously received at least one dose of the multispecific antibody or antigen-binding fragment thereof, comprising administering at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a method of treatment and/or prevention of atopic dermatitis in a subject in need thereof, wherein the subject has received at least one loading dose of the multispecific antibody or antigen-binding fragment thereof, comprising administering at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the manufacture of a medicament for the treatment and/or prevention of atopic dermatitis in a subject in need thereof, wherein the subject has previously received at least one dose of the multispecific antibody or antigen-binding fragment thereof, wherein at least one maintenance dose is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the manufacture of a medicament for the treatment and/or prevention of atopic dermatitis in a subject in need thereof, wherein the subject has received at least one loading dose of the multispecific antibody or antigen-binding fragment thereof, wherein at least one maintenance dose is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the treatment and/or prevention of atopic dermatitis in a subject in need thereof, wherein the subject has previously received at least one dose of the multispecific antibody or antigen-binding fragment thereof, wherein least one maintenance dose is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In a further aspect, there is provided a use of a multispecific antibody or antigen-binding fragment thereof for the treatment and/or prevention of atopic dermatitis in a subject in need thereof, wherein the subject has received at least one loading dose of the multispecific antibody or antigen-binding fragment thereof, wherein least one maintenance dose is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

In preferred embodiments, the multispecific antibody is bispecific. In some embodiments, the multispecific antibody is an IL-18 and IL-13 antagonist. In some embodiments, the first part that binds to IL-18 comprises a first variable heavy region (VH1) comprising SEQ ID NO: 41 and a first variable light region (VL1) comprising SEQ ID NO: 13, and the second part that binds to IL-13 comprises a second variable heavy region (VH2) comprising by SEQ ID NO: 55 and a second variable light region (VL2) comprising by SEQ ID NO: 27.

In some embodiments, the antibody is bispecific and comprises a first part that binds to IL-18 comprising or consisting of a first heavy chain (HC1) comprising or consisting of SEQ ID NO: 42 and a first light chain (LC1) comprising or consisting of SEQ ID NO: 14, and a second part that binds to IL-13 comprising or consisting of a second heavy chain (HC2) comprising or consisting of SEQ ID NO: 56 and a second light chain (LC2) comprising or consisting of SEQ ID NO: 28. In some embodiments, the antibody is bispecific and comprises a first part that binds to IL-18 comprising or consisting of a first heavy chain (HC1) comprising or consisting of SEQ ID NO: 57 and a first light chain (LC1) comprising or consisting of SEQ ID NO: 14, and a second part that binds to IL-13 comprising or consisting of a second heavy chain (HC2) comprising or consisting of SEQ ID NO: 58 and a second light chain (LC2) comprising or consisting of SEQ ID NO: 28. In preferred embodiments, the atopic dermatitis is moderate-to-severe atopic dermatitis. In some embodiments, the at least one maintenance dose is administered subcutaneously or intravenously, preferably subcutaneously. In some embodiments, the at least one maintenance dose comprises an amount of the multispecific antibody or antigen-binding fragment thereof selected from the group consisting of about 60 mg, about 150 mg, about 300 mg, and about 600 mg. In some embodiments, maintenance dose administration frequency is selected from the group consisting of once a week (Q1W), once every two weeks (Q2W), once every four weeks (Q4W), one every eight weeks (Q8W), once every twelve weeks (Q12W), and a combination thereof. In some embodiments, each maintenance dose comprises about 60 mg of the multispecific antibody or antigen-binding fragment thereof. In some embodiments, each maintenance dose comprises about 150 mg of the multispecific antibody or antigen-binding fragment thereof. In some embodiments, each maintenance dose comprises about 300 mg of the multispecific antibody or antigen-binding fragment thereof. In some embodiments, each maintenance dose comprises about 600 mg of the multispecific antibody or antigen-binding fragment thereof. In some embodiments, the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks. In some embodiments, the subject has received at least three (e.g., three) loading doses of the multispecific antibody or antigen-binding fragment thereof. In some embodiments, a maintenance dose is administered two weeks, four weeks, eight weeks, or twelve weeks after the last loading dose has been administered to the subject.

In a further aspect, there is provided a method of treatment and/or prevention of atopic dermatitis in a subject in need thereof, comprising administering a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
and wherein the antibody is administered subcutaneously at a dosing regimen selected from the group consisting of about150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a multispecific antibody or antigen-binding fragment thereof for use in the treatment and/or prevention of atopic dermatitis in a subject in need thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
and wherein the antibody is administered subcutaneously at a dosing regimen selected from the group consisting of about150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof for the manufacture of a medicament for the treatment and/or prevention of atopic dermatitis in a subject in need thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
and wherein the antibody is administered subcutaneously at a dosing regimen selected from the group consisting of about150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In a further aspect, there is provided a use of a multispecific antibody or antigen-binding fragment thereof in the treatment and/or prevention of atopic dermatitis in a subject in need thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
and wherein the antibody is administered subcutaneously at a dosing regimen selected from the group consisting of about150 mg Q2W, about 150 mg Q4W, about 150 mg Q8W, about 150 mg Q12W, about 300 mg Q2W, about 300 mg Q4W, about 300 mg Q8W, about 300 mg Q12W, about 600 mg Q2W, about 600 mg Q4W, about 600 mg Q8W, and about 600 mg Q12W.

In some embodiments, the dosing regimen is selected from the group consisting of about 300 mg Q2W, about 300 mg Q4W, about 300 mg Q8W, about 300 mg Q12W, about 600 mg Q2W, about 600 mg Q4W, about 600 mg Q8W, and about 600 mg Q12W, among which about 300 mg Q4W and about 600 mg Q4W are preferred. In some embodiments, the dosing regimen is about 300 mg Q2W. In some embodiments, the dosing regimen is about 300 mg Q4W. In some embodiments, the dosing regimen is about 300 mg Q8W. In some embodiments, the dosing regimen is about 300 mg Q12W. In some embodiments, the dosing regimen is about 600 mg Q2W. In some embodiments, the dosing regimen is about 600 mg Q4W. In some embodiments, the dosing regimen is about 600 mg Q8W. In some embodiments, the dosing regimen is about 600 mg Q12W.

In preferred embodiments, the multispecific antibody is bispecific. In some embodiments, the multispecific antibody is an IL-18 and IL-13 antagonist. In some embodiments, the first part that binds to IL-18 comprises a first variable heavy region (VH1) comprising SEQ ID NO: 41 and a first variable light region (VL1) comprising SEQ ID NO: 13, and the second part that binds to IL-13 comprises a second variable heavy region (VH2) comprising by SEQ ID NO: 55 and a second variable light region (VL2) comprising by SEQ ID NO: 27.

In some embodiments, the antibody is bispecific and comprises a first part that binds to IL-18 comprising or consisting of a first heavy chain (HC1) comprising or consisting of SEQ ID NO: 42 and a first light chain (LC1) comprising or consisting of SEQ ID NO: 14, and a second part that binds to IL-13 comprising or consisting of a second heavy chain (HC2) comprising or consisting of SEQ ID NO: 56 and a second light chain (LC2) comprising or consisting of SEQ ID NO: 28. In some embodiments, the antibody is bispecific and comprises a first part that binds to IL-18 comprising or consisting of a first heavy chain (HC1) comprising or consisting of SEQ ID NO: 57 and a first light chain (LC1) comprising or consisting of SEQ ID NO: 14, and a second part that binds to IL-13 comprising or consisting of a second heavy chain (HC2) comprising or consisting of SEQ ID NO: 58 and a second light chain (LC2) comprising or consisting of SEQ ID NO: 28. In preferred embodiments, the atopic dermatitis is moderate-to-severe atopic dermatitis.

### Enumerated Embodiments

Embodiment 1. A multispecific antibody or antigen-binding fragment thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
for use in the treatment and/or prevention of atopic dermatitis in a subject in need thereof, wherein at least one loading dose and at least one maintenance dose are administered to the subject.

Embodiment 2. The multispecific antibody or antigen-binding fragment thereof for use according to Embodiment 1, wherein the antibody is bispecific.

Embodiment 3. The multispecific antibody or antigen-binding fragment thereof for use according to Embodiment 1 or 2, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 4. The multispecific antibody or antigen-binding fragment thereof for use according to any one of Embodiments 1 to 3, wherein the first part that binds to IL-18 comprises a first light chain (LC1) that is a lambda light chain and the second part that binds to IL-13 comprises a second light chain (LC2) that is a kappa light chain.

Embodiment 5. The multispecific antibody or antigen-binding fragment thereof for use according to any one of Embodiments 1 to 4, wherein the first part that binds to IL-18 comprises a first light chain (LC1) comprising SEQ ID NO: 14 and the second part that binds to IL-13 comprises a second light chain (LC2) comprising SEQ ID NO: 28.

Embodiment 6. The multispecific antibody or antigen-binding fragment thereof according to any one of Embodiments 1 to 5, wherein:
the first part that binds to IL-18 comprises a first Fc polypeptide, and
the second part that binds to IL-13 comprises a second Fc polypeptide, wherein the first and/or second Fc polypeptide comprises the amino acid sequence SEQ ID NO: 59 or a variant thereof.

Embodiment 7. The multispecific antibody or antigen-binding fragment thereof for use according to Embodiment 6, wherein the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution,
according to EU numbering.

Embodiment 8. The multispecific antibody or antigen-binding fragment thereof for use according to Embodiment 6 or 7, wherein:
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
   or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution, according to EU numbering.

Embodiment 9. The multispecific antibody or antigen-binding fragment thereof for use according to any one of Embodiments 1 to 8, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 10. The multispecific antibody or antigen-binding fragment thereof for use according to any one of Embodiments 1 to 8, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 11. The multispecific antibody or antigen-binding fragment thereof for use according to any one of Embodiments 1 to 10, wherein the IL-18 and IL-13 are human.

Embodiment 12. The multispecific antibody or antigen-binding fragment thereof for use according to any one of Embodiments 1 to 11, wherein the subject is a human.

Embodiment 13. The multispecific antibody or antigen-binding fragment thereof for use according to any one of Embodiments 1 to 12, wherein the atopic dermatitis is moderate-to-severe atopic dermatitis.

Embodiment 14. The multispecific antibody or antigen-binding fragment thereof for use according to any one of Embodiments 1 to 13, wherein the at least one loading dose is administered subcutaneously or intravenously, preferably subcutaneously.

Embodiment 15. The multispecific antibody or antigen-binding fragment thereof for use according to any one of Embodiments 1 to 14, wherein the at least one maintenance dose is administered subcutaneously or intravenously, preferably subcutaneously.

Embodiment 16. The multispecific antibody or antigen-binding fragment thereof for use according to any one of Embodiments 1 to 15, wherein the at least one loading dose and/or the at least one maintenance dose comprise an amount of the multispecific antibody or antigen-binding fragment thereof sufficient to achieve a therapeutically effective serum level.

Embodiment 17. The multispecific antibody or antigen-binding fragment thereof for use according to Embodiment 16, wherein the therapeutically effective serum level is maintained throughout the course of the treatment.

Embodiment 18. The multispecific antibody or antigen-binding fragment thereof for use according to any one of Embodiments 1 to 17, wherein the at least one loading dose comprises an amount of the multispecific antibody or antigen-binding fragment thereof selected from the group consisting of about 60 mg, about 150 mg, about 300 mg, and about 600 mg.

Embodiment 19. The multispecific antibody or antigen-binding fragment thereof for use according to any one of Embodiments 1 to 18, wherein the at least one maintenance dose comprises an amount of the multispecific antibody or antigen-binding fragment thereof selected from the group consisting of about 60 mg, about 150 mg, about 300 mg, and about 600 mg.

Embodiment 20. The multispecific antibody or antigen-binding fragment thereof for use according to any one of Embodiments 1 to 19, wherein at least two or at least three loading doses are administered.

Embodiment 21. The multispecific antibody or antigen-binding fragment thereof for use according to Embodiment 20, wherein a first loading dose is administered on day 1, a second loading dose is administered on week 1, and a third loading dose is administered on week 2 of the treatment.

Embodiment 22. The multispecific antibody or antigen-binding fragment thereof for use according to any one of Embodiments 1 to 21, wherein at least two, at least three, at least four, at least five, at least six, at least seven, at least eight, at least nine, at least ten, at least eleven, or at least twelve maintenance doses are administered.

Embodiment 23. The multispecific antibody or antigen-binding fragment thereof for use according to any one of Embodiments 1 to 22, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 24. The multispecific antibody or antigen-binding fragment thereof for use according to any one of Embodiments 1 to 23, wherein at least two maintenance doses are administered, and wherein the first maintenance dose is administered on week 4 of the treatment, followed by administration once every two weeks (Q2W), once every four weeks (Q4W), once every eight weeks (Q8W), or once every twelve weeks (Q12W).

Embodiment 25. The multispecific antibody or antigen-binding fragment thereof for use according to any one of Embodiments 1 to 24, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 26. The multispecific antibody or antigen-binding fragment thereof for use according to any one of Embodiments 1 to 25, wherein at least one, at least two, or at least three (e.g., three) loading doses comprising about 60 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the multispecific antibody or antigen-binding fragment thereof are administered.

Embodiment 27. The multispecific antibody or antigen-binding fragment thereof for use according to any one of Embodiments 1 to 25, wherein at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

Embodiment 28. The multispecific antibody or antigen-binding fragment thereof for use according to any one of Embodiments 1 to 25 or 27, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 150 mg of the multispecific antibody or antigen-binding fragment thereof.

Embodiment 29. The multispecific antibody or antigen-binding fragment thereof for use according to Embodiment 28, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 30. The multispecific antibody or antigen-binding fragment thereof for use according to Embodiment 28, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 31. The multispecific antibody or antigen-binding fragment thereof for use according to any one of Embodiments 1 to 25, wherein at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

Embodiment 32. The multispecific antibody or antigen-binding fragment thereof for use according to any one of Embodiments 1 to 25 or 31, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 300 mg of the multispecific antibody or antigen-binding fragment thereof.

Embodiment 33. The multispecific antibody or antigen-binding fragment thereof for use according to Embodiment 32, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 34. The multispecific antibody or antigen-binding fragment thereof for use according to Embodiment 32, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 35. The multispecific antibody or antigen-binding fragment thereof for use according to any one of Embodiments 1 to 25, wherein at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered.

Embodiment 36. The multispecific antibody or antigen-binding fragment thereof for use according to any one of Embodiments 1 to 25 or 35, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 600 mg of the multispecific antibody or antigen-binding fragment thereof.

Embodiment 37. The multispecific antibody or antigen-binding fragment thereof for use according to Embodiment 36, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 38. The multispecific antibody or antigen-binding fragment thereof for use according to Embodiment 36, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 39. The multispecific antibody or antigen-binding fragment thereof for use according to any one of Embodiments 1 to 38 wherein the subject, at baseline, has one or more of the following:
an Eczema Area and Severity Index (EASI) score of ≥12 or ≥16, preferably ≥16,
an Investigator's Global Assessment (IGA) score of ≥3, and/or
a Body Surface Area (BSA) of ≥10%.

Embodiment 40. The multispecific antibody or antigen-binding fragment thereof for use according to any one of Embodiments 1 to 39, wherein the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI) score;
Peak Pruritus Numeric Rating scale (PP-NRS) score, and;
a combination thereof.

Embodiment 41. The multispecific antibody or antigen-binding fragment thereof for use according to any one of Embodiments 1 to 40, wherein the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment.

Embodiment 42. The multispecific antibody or antigen-binding fragment thereof for use according to any one of Embodiments 1 to 41, wherein the treatment results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP).

Embodiment 43. The multispecific antibody or antigen-binding fragment thereof for use according to any one of Embodiments 1 to 42, wherein the subject has a history of inadequate response to treatment with a topical medication or is a subject for whom topical medications are medically inadvisable.

Embodiment 44. The multispecific antibody or antigen-binding fragment thereof for use according to Embodiment 43, wherein the topical medication is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist.

Embodiment 45. The multispecific antibody or antigen-binding fragment thereof for use according to Embodiment 44, wherein the topical corticosteroid is selected from the group consisting of methylprednisolone aceponate, mometasone furoate, fluticasone propionate, betamethasone valerate, triamcinolone, and hydrocortisone butyrate.

Embodiment 46. The multispecific antibody or antigen-binding fragment thereof for use according to any one of Embodiments 1 to 45, wherein the treatment results in alleviation of a symptom associated with atopic dermatitis relative to baseline, preferably selected from the group consisting of erythema, induration/papulation, excoriations, lichenification, pruritus, dry skin, thickened skin, cracked skin, scaly skin, raw skin, skin sensitivity, swollen skin, scaly and dry eczematous lesions, and a combination thereof.

Embodiment 47. The multispecific antibody or antigen-binding fragment thereof for use according to any one of Embodiments 1 to 46, wherein the treatment comprises administration of at least one additional therapeutic agent.

Embodiment 48. The multispecific antibody or antigen-binding fragment thereof for use according to Embodiment 47, wherein the at least one additional therapeutic agent is administered concurrently or sequentially with the multispecific antibody or antigen-binding fragment thereof.

Embodiment 49. The multispecific antibody or antigen-binding fragment thereof for use according to Embodiment 48, wherein the additional therapeutic agent is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

Embodiment 50. The multispecific antibody or antigen-binding fragment thereof for use according to any one of Embodiments 1 to 49, wherein the multispecific antibody or antigen-binding fragment thereof is comprised in a pharmaceutical composition, wherein the pharmaceutical composition optionally comprises one or more pharmaceutically acceptable ingredients.

Embodiment 51. The multispecific antibody or antigen-binding fragment thereof for use according to Embodiment 50, wherein the pharmaceutical composition is comprised in a kit comprising one or more doses of the pharmaceutical composition, optionally together with instructions for use.

Embodiment 52. A multispecific antibody or antigen-binding fragment thereof, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26,
for use in the treatment and/or prevention of atopic dermatitis in a subject in need thereof, wherein the subject has received at least one loading dose of the multispecific antibody or antigen-binding fragment thereof, and wherein at least one maintenance dose is administered to the subject.

Embodiment 53. The multispecific antibody or antigen-binding fragment thereof for use according to Embodiment 52, wherein the antibody is bispecific.

Embodiment 54. The multispecific antibody or antigen-binding fragment thereof for use according to Embodiment 52 or 53, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 55. The multispecific antibody or antigen-binding fragment thereof for use according to any one of Embodiments 52 to 54, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 56. The multispecific antibody or antigen-binding fragment thereof for use according to any one of Embodiments 52 to 55, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 57. The multispecific antibody or antigen-binding fragment thereof for use according to any one of Embodiments 52 to 56, wherein the atopic dermatitis is moderate-to-severe atopic dermatitis.

Embodiment 58. The multispecific antibody or antigen-binding fragment thereof for use according to any one of Embodiments 52 to 57, wherein the at least one maintenance dose is administered subcutaneously or intravenously, preferably subcutaneously.

Embodiment 59. The multispecific antibody or antigen-binding fragment thereof for use according to any one of Embodiments 52 to 58, wherein the at least one maintenance dose comprises an amount of the multispecific antibody or antigen-binding fragment thereof selected from the group consisting of about 60 mg, about 150 mg, about 300 mg, and about 600 mg.

Embodiment 60. The multispecific antibody or antigen-binding fragment thereof for use according to any one of Embodiments 52 to 59, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 61. The multispecific antibody or antigen-binding fragment thereof for use according to Embodiment 60, wherein each maintenance dose comprises about 60 mg of the multispecific antibody or antigen-binding fragment thereof.

Embodiment 62. The multispecific antibody or antigen-binding fragment thereof for use according to Embodiment 60, wherein each maintenance dose comprises about 150 mg of the multispecific antibody or antigen-binding fragment thereof.

Embodiment 63. The multispecific antibody or antigen-binding fragment thereof for use according to Embodiment 60, wherein each maintenance dose comprises about 300 mg of the multispecific antibody or antigen-binding fragment thereof.

Embodiment 64. The multispecific antibody or antigen-binding fragment thereof for use according to Embodiment 60, wherein each maintenance dose comprises about 600 mg of the multispecific antibody or antigen-binding fragment thereof.

Embodiment 65. The multispecific antibody or antigen-binding fragment thereof for use according to any one of Embodiments 52 to 64, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 66. The multispecific antibody or antigen-binding fragment thereof for use according to any one of Embodiments 52 to 65, wherein the subject has received at least three loading doses of the multispecific antibody or antigen-binding fragment thereof.

Embodiment 67. The multispecific antibody or antigen-binding fragment thereof for use according to any one of Embodiments 52 to 66, wherein a maintenance dose is administered two weeks, four weeks, eight weeks, or twelve weeks after the last loading dose has been administered to the subject.

Embodiment 68. A method of treatment and/or prevention of atopic dermatitis in a subject in need thereof, comprising administering at least one loading dose and at least one maintenance dose of a multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

Embodiment 69. The method according to Embodiment 68, wherein the antibody is bispecific.

Embodiment 70. The method according to Embodiment 68 or 69, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 71. The method according to any one of Embodiments 68 to 70, wherein the first part that binds to IL-18 comprises a first light chain (LC1) that is a lambda light chain and the second part that binds to IL-13 comprises a second light chain (LC2) that is a kappa light chain.

Embodiment 72. The method according to any one of Embodiments 68 to 71, wherein the first part that binds to IL-18 comprises a first light chain (LC1) comprising SEQ ID NO: 14 and the second part that binds to IL-13 comprises a second light chain (LC2) comprising SEQ ID NO: 28.

Embodiment 73. The method according to any one of Embodiments 68 to 72, wherein:
the first part that binds to IL-18 comprises a first Fc polypeptide, and
the second part that binds to IL-13 comprises a second Fc polypeptide, wherein the first and/or second Fc polypeptide comprises the amino acid sequence SEQ ID NO: 59 or a variant thereof.

Embodiment 74. The method according to Embodiment 73, wherein the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution,
according to EU numbering.

Embodiment 75. The method according to Embodiment 73 or 74, wherein:
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
   or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution,
   according to EU numbering.

Embodiment 76. The method according to any one of Embodiments 68 to 75, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 77. The method according to any one of Embodiments 68 to 75, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 78. The method according to any one of Embodiments 68 to 77, wherein the IL-18 and IL-13 are human.

Embodiment 79. The method according to any one of Embodiments 68 to 78, wherein the subject is a human.

Embodiment 80. The method according to any one of Embodiments 68 to 79, wherein the atopic dermatitis is moderate-to-severe atopic dermatitis.

Embodiment 81. The method according to any one of Embodiments 68 to 80, wherein the at least one loading dose is administered subcutaneously or intravenously, preferably subcutaneously.

Embodiment 82. The method according to any one of Embodiments 68 to 81, wherein the at least one maintenance dose is administered subcutaneously or intravenously, preferably subcutaneously.

Embodiment 83. The method according to any one of Embodiments 68 to 82, wherein the at least one loading dose and/or the at least one maintenance dose comprise an amount of the multispecific antibody or antigen-binding fragment thereof sufficient to achieve a therapeutically effective serum level.

Embodiment 84. The method according to Embodiment 83, wherein the therapeutically effective serum level is maintained throughout the course of the treatment.

Embodiment 85. The method according to any one of Embodiments 68 to 84, wherein the at least one loading dose comprises an amount of the multispecific antibody or antigen-binding fragment thereof selected from the group consisting of about 60 mg, about 150 mg, about 300 mg, and about 600 mg.

Embodiment 86. The method according to any one of Embodiments 68 to 85, wherein the at least one maintenance dose comprises an amount of the multispecific antibody or antigen-binding fragment thereof selected from the group consisting of about 60 mg, about 150 mg, about 300 mg, and about 600 mg.

Embodiment 87. The method according to any one of Embodiments 68 to 86, wherein at least two or at least three loading doses are administered.

Embodiment 88. The method according to Embodiment 87, wherein a first loading dose is administered on day 1, a second loading dose is administered on week 1, and a third loading dose is administered on week 2 of the treatment.

Embodiment 89. The method according to any one of Embodiments 68 to 88, wherein at least two, at least three, at least four, at least five, at least six, at least seven, at least eight, at least nine, at least ten, at least eleven, or at least twelve maintenance doses are administered.

Embodiment 90. The method according to any one of Embodiments 68 to 89, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 91. The method according to any one of Embodiments 68 to 90, wherein at least two maintenance doses are administered, and wherein the first maintenance dose is administered on week 4 of the treatment, followed by administration once every two weeks (Q2W), once every four weeks (Q4W), once every eight weeks (Q8W), or once every twelve weeks (Q12W).

Embodiment 92. The method according to any one of Embodiments 68 to 91, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 93. The method according to any one of Embodiments 68 to 92, wherein at least one, at least two, or at least three (e.g., three) loading doses comprising about 60 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the multispecific antibody or antigen-binding fragment thereof are administered.

Embodiment 94. The method according to any one of Embodiments 68 to 92, wherein at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

Embodiment 95. The method according to any one of Embodiments 68 to 92 or 94, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 150 mg of the multispecific antibody or antigen-binding fragment thereof.

Embodiment 96. The method according to Embodiment 95, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 97. The method according to Embodiment 95, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 98. The method according to any one of Embodiments 68 to 92, wherein at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

Embodiment 99. The method according to any one of Embodiments 68 to 92 or 98, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 300 mg of the multispecific antibody or antigen-binding fragment thereof.

Embodiment 100. The method according to Embodiment 99, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 101. The method according to Embodiment 99, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 102. The method according to any one of Embodiments 68 to 92, wherein at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered. Embodiment 103. The method according to any one of Embodiments 68 to 92 or 102, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 600 mg of the multispecific antibody or antigen-binding fragment thereof.

Embodiment 104. The method according to Embodiment 103, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 105. The method according to Embodiment 103, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 106. The method according to any one of Embodiments 68 to 105 wherein the subject, at baseline, has one or more of the following:
an Eczema Area and Severity Index (EASI) score of ≥12 or ≥16, preferably ≥16,
an Investigator's Global Assessment (IGA) score of ≥3, and/or
a Body Surface Area (BSA) of ≥10%.

Embodiment 107. The method according to any one of Embodiments 68 to 106, wherein the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI) score;
Peak Pruritus Numeric Rating scale (PP-NRS) score, and;
a combination thereof.

Embodiment 108. The method according to any one of Embodiments 68 to 107, wherein the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment.

Embodiment 109. The method according to any one of Embodiments 68 to 108, wherein the treatment results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP).

Embodiment 110. The method according to any one of Embodiments 68 to 109, wherein the subject has a history of inadequate response to treatment with a topical medication or is a subject for whom topical medications are medically inadvisable.

Embodiment 111. The method according to Embodiment 110, wherein the topical medication is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist.

Embodiment 112. The method according to Embodiment 111, wherein the topical corticosteroid is selected from the group consisting of methylprednisolone aceponate, mometasone furoate, fluticasone propionate, betamethasone valerate, triamcinolone, and hydrocortisone butyrate.

Embodiment 113. The method according to any one of Embodiments 68 to 112, wherein the treatment results in alleviation of a symptom associated with atopic dermatitis relative to baseline, preferably selected from the group consisting of erythema, induration/papulation, excoriations, lichenification, pruritus, dry skin, thickened skin, cracked skin, scaly skin, raw skin, skin sensitivity, swollen skin, scaly and dry eczematous lesions, and a combination thereof.

Embodiment 114. The method according to any one of Embodiments 68 to 113, wherein the treatment comprises administration of at least one additional therapeutic agent.

Embodiment 115. The method according to Embodiment 114, wherein the at least one additional therapeutic agent is administered concurrently or sequentially with the multispecific antibody or antigen-binding fragment thereof.

Embodiment 116. The method according to Embodiment 115, wherein the additional therapeutic agent is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

Embodiment 117. The method according to any one of Embodiments 68 to 116, wherein the multispecific antibody or antigen-binding fragment thereof is comprised in a pharmaceutical composition, wherein the pharmaceutical composition optionally comprises one or more pharmaceutically acceptable ingredients.

Embodiment 118. The method according to Embodiment 117, wherein the pharmaceutical composition is comprised in a kit comprising one or more doses of the pharmaceutical composition, optionally together with instructions for use.

Embodiment 119. A method of treatment and/or prevention of atopic dermatitis in a subject in need thereof, wherein the subject has received at least one loading dose of a multispecific antibody or antigen-binding fragment thereof, comprising administering at least one maintenance dose of the multispecific antibody or antigen-binding fragment thereof to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

Embodiment 120. The method according to Embodiment 119, wherein the antibody bispecific.

Embodiment 121. The method according to Embodiment 119 or 120, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 122. The method according to any one of Embodiments 119 to 121, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 123. The method according to any one of Embodiments 119 to 121, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 124. The method according to any one of Embodiments 119 to 123, wherein the atopic dermatitis is moderate-to-severe atopic dermatitis.

Embodiment 125. The method according to any one of Embodiments 119 to 124, wherein the at least one maintenance dose is administered subcutaneously or intravenously, preferably subcutaneously.

Embodiment 126. The method according to any one of Embodiments 119 to 125, wherein the at least one maintenance dose comprises an amount of the multispecific antibody or antigen-binding fragment thereof selected from the group consisting of about 60 mg, about 150 mg, about 300 mg, and about 600 mg.

Embodiment 127. The method according to any one of Embodiments 119 to 126, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 128. The method according to Embodiment 127, wherein each maintenance dose comprises about 60 mg of the multispecific antibody or antigen-binding fragment thereof.

Embodiment 129. The method according to Embodiment 127, wherein each maintenance dose comprises about 150 mg of the multispecific antibody or antigen-binding fragment thereof.

Embodiment 130. The method according to Embodiment 127, wherein each maintenance dose comprises about 300 mg of the multispecific antibody or antigen-binding fragment thereof.

Embodiment 131. The method according to Embodiment 127, wherein each maintenance dose comprises about 600 mg of the multispecific antibody or antigen-binding fragment thereof.

Embodiment 132. The method according to any one of Embodiments 119 to 131, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 133. The method according to any one of Embodiments 119 to 132, wherein the subject has received at least three loading doses of the multispecific antibody or antigen-binding fragment thereof.

Embodiment 134. The method according to any one of Embodiments 119 to 133, wherein a maintenance dose is administered two weeks, four weeks, eight weeks, or twelve weeks after the last loading dose has been administered to the subject.

Embodiment 135. A use of a multispecific antibody or antigen-binding fragment thereof for the manufacture of a medicament for the treatment and/or prevention of atopic dermatitis in a subject in need thereof, wherein at least one loading dose and at least one maintenance dose of the multispecific antibody or antigen-binding fragment thereof are administered to the subject, wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

Embodiment 136. The use according to Embodiment 135, wherein the antibody is bispecific.

Embodiment 137. The use according to Embodiment 135 or 136, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 138. The use according to any one of Embodiments 135 to 137, wherein the first part that binds to IL-18 comprises a first light chain (LC1) that is a lambda light chain and the second part that binds to IL-13 comprises a second light chain (LC2) that is a kappa light chain.

Embodiment 139. The use according to any one of Embodiments 135 to 138, wherein the first part that binds to IL-18 comprises a first light chain (LC1) comprising SEQ ID NO: 14 and the second part that binds to IL-13 comprises a second light chain (LC2) comprising SEQ ID NO: 28.

Embodiment 140. The use according to any one of Embodiments 135 to 139, wherein:
the first part that binds to IL-18 comprises a first Fc polypeptide, and
the second part that binds to IL-13 comprises a second Fc polypeptide, wherein the first and/or second Fc polypeptide comprises the amino acid sequence SEQ ID NO: 59 or a variant thereof.

Embodiment 141. The use according to Embodiment 140, wherein the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution,
according to EU numbering.

Embodiment 142. The use according to Embodiment 140 or 141, wherein:
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
   or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution,
   according to EU numbering.

Embodiment 143. The use according to any one of Embodiments 135 to 142, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 144. The use according to any one of Embodiments 135 to 142, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 145. The use according to any one of Embodiments 135 to 144, wherein the IL-18 and IL-13 are human.

Embodiment 146. The use according to any one of Embodiments 135 to 145, wherein the subject is a human.

Embodiment 147. The use according to any one of Embodiments 135 to 146, wherein the atopic dermatitis is moderate-to-severe atopic dermatitis.

Embodiment 148. The use according to any one of Embodiments 135 to 147, wherein the at least one loading dose is administered subcutaneously or intravenously, preferably subcutaneously.

Embodiment 149. The use according to any one of Embodiments 135 to 148, wherein the at least one maintenance dose is administered subcutaneously or intravenously, preferably subcutaneously.

Embodiment 150. The use according to any one of Embodiments 135 to 149, wherein the at least one loading dose and/or the at least one maintenance dose comprise an amount of the multispecific antibody or antigen-binding fragment thereof sufficient to achieve a therapeutically effective serum level.

Embodiment 151. The use according to Embodiment 150, wherein the therapeutically effective serum level is maintained throughout the course of the treatment.

Embodiment 152. The use according to any one of Embodiments 135 to 151, wherein the at least one loading dose comprises an amount of the multispecific antibody or antigen-binding fragment thereof selected from the group consisting of about 60 mg, about 150 mg, about 300 mg, and about 600 mg.

Embodiment 153. The use according to any one of Embodiments 135 to 152, wherein the at least one maintenance dose comprises an amount of the multispecific antibody or antigen-binding fragment thereof selected from the group consisting of about 60 mg, about 150 mg, about 300 mg, and about 600 mg.

Embodiment 154. The use according to any one of Embodiments 135 to 153, wherein at least two or at least three loading doses are administered.

Embodiment 155. The use according to Embodiment 154, wherein a first loading dose is administered on day 1, a second loading dose is administered on week 1, and a third loading dose is administered on week 2 of the treatment.

Embodiment 156. The use according to any one of Embodiments 135 to 155, wherein at least two, at least three, at least four, at least five, at least six, at least seven, at least eight, at least nine, at least ten, at least eleven, or at least twelve maintenance doses are administered.

Embodiment 157. The use according to any one of Embodiments 135 to 156, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 158. The use according to any one of Embodiments 135 to 157, wherein at least two maintenance doses are administered, and wherein the first maintenance dose is administered on week 4 of the treatment, followed by administration once every two weeks (Q2W), once every four weeks (Q4W), once every eight weeks (Q8W), or once every twelve weeks (Q12W).

Embodiment 159. The use according to any one of Embodiments 135 to 158, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 160. The use according to any one of Embodiments 135 to 159, wherein at least one, at least two, or at least three (e.g., three) loading doses comprising about 60 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 60 mg of the multispecific antibody or antigen-binding fragment thereof are administered.

Embodiment 161. The use according to any one of Embodiments 135 to 159, wherein at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

Embodiment 162. The use according to any one of Embodiments 135 to 159 or 161, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 150 mg of the multispecific antibody or antigen-binding fragment thereof.

Embodiment 163. The use according to Embodiment 162, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 164. The use according to Embodiment 162, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 165. The use according to any one of Embodiments 135 to 159, wherein at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

Embodiment 166. The use according to any one of Embodiments 135 to 159 or 165, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 300 mg of the multispecific antibody or antigen-binding fragment thereof.

Embodiment 167. The use according to Embodiment 166, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 168. The use according to Embodiment 166, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 169. The use according to any one of Embodiments 135 to 159, wherein at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered.

Embodiment 170. The use according to any one of Embodiments 135 to 159 or 169, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 600 mg of the multispecific antibody or antigen-binding fragment thereof.

Embodiment 171. The use according to Embodiment 170, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 172. The use according to Embodiment 170, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

Embodiment 173. The use according to any one of Embodiments 135 to 172 wherein the subject, at baseline, has one or more of the following:
an Eczema Area and Severity Index (EASI) score of ≥12 or ≥16, preferably ≥16,
an Investigator's Global Assessment (IGA) score of ≥3, and/or
a Body Surface Area (BSA) of ≥10%.

Embodiment 174. The use according to any one of Embodiments 135 to 173, wherein the treatment results in an improvement relative to baseline in an atopic dermatitis-associated parameter selected from the group consisting of:
Investigator's Global Assessment (IGA) score;
Eczema Area and Severity Index (EASI) score;
Body Surface Area (BSA);
Dermatology Life Quality Index (DLQI) score;
Peak Pruritus Numeric Rating scale (PP-NRS) score, and;
a combination thereof.

Embodiment 175. The use according to any one of Embodiments 135 to 174, wherein the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment.

Embodiment 176. The use according to any one of Embodiments 135 to 175, wherein the treatment results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP).

Embodiment 177. The use according to any one of Embodiments 135 to 176, wherein the subject has a history of inadequate response to treatment with a topical medication or is a subject for whom topical medications are medically inadvisable.

Embodiment 178. The use according to Embodiment 177, wherein the topical medication is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist.

Embodiment 179. The use according to Embodiment 178, wherein the topical corticosteroid is selected from the group consisting of methylprednisolone aceponate, mometasone furoate, fluticasone propionate, betamethasone valerate, triamcinolone, and hydrocortisone butyrate.

Embodiment 180. The use according to any one of Embodiments 135 to 179, wherein the treatment results in alleviation of a symptom associated with atopic dermatitis relative to baseline, preferably selected from the group consisting of erythema, induration/papulation, excoriations, lichenification, pruritus, dry skin, thickened skin, cracked skin, scaly skin, raw skin, skin sensitivity, swollen skin, scaly and dry eczematous lesions, and a combination thereof.

Embodiment 181. The use according to any one of Embodiments 135 to 180, wherein the treatment comprises administration of at least one additional therapeutic agent.

Embodiment 182. The use according to Embodiment 181, wherein the at least one additional therapeutic agent is administered concurrently or sequentially with the multispecific antibody or antigen-binding fragment thereof.

Embodiment 183. The use according to Embodiment 182, wherein the additional therapeutic agent is selected from the group consisting of a topical steroid, e.g., corticosteroid, a calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, an aryl hydrocarbon receptor (AHR) agonist, a JAK inhibitor, dupilumab, lebrikizumab, tralokinumab, etanercept, adalimumab, infliximab, omalizumab, and secukinumab.

Embodiment 184. The use according to any one of Embodiments 135 to 183, wherein the multispecific antibody or antigen-binding fragment thereof is comprised in a pharmaceutical composition, wherein the pharmaceutical composition optionally comprises one or more pharmaceutically acceptable ingredients.

Embodiment 185. The use according to Embodiment 184, wherein the pharmaceutical composition is comprised in a kit comprising one or more doses of the pharmaceutical composition, optionally together with instructions for use.

Embodiment 186. A use of a multispecific antibody or antigen-binding fragment thereof for the manufacture of a medicament for the treatment and/or prevention of atopic dermatitis in a subject in need thereof, wherein the subject has received at least one loading dose of a multispecific antibody or antigen-binding fragment thereof, wherein at least one maintenance dose of the multispecific antibody or antigen-binding fragment thereof is administered to the subject, and wherein the antibody comprises:
i) a first part that binds to IL-18 comprising:
   an HCDR1 comprising SEQ ID NO: 29, SEQ ID NO: 32, SEQ ID NO: 35, or SEQ ID NO: 38;
   an HCDR2 comprising SEQ ID NO: 30, SEQ ID NO: 33, SEQ ID NO: 36, or SEQ ID NO: 39;
   an HCDR3 comprising SEQ ID NO: 31, SEQ ID NO: 34, SEQ ID NO: 37, or SEQ ID NO: 40;
   an LCDR1 comprising SEQ ID NO: 1, SEQ ID NO: 4, SEQ ID NO: 7, or SEQ ID NO: 10;
   an LCDR2 comprising SEQ ID NO: 2, SEQ ID NO: 5, or amino acid sequence RNN, and;
   an LCDR3 comprising SEQ ID NO: 3, SEQ ID NO: 6, SEQ ID NO: 9, or SEQ ID NO: 12, and;
ii) a second part that binds to IL-13 comprising:
   an HCDR1 comprising SEQ ID NO: 43, SEQ ID NO: 46, SEQ ID NO: 49, or SEQ ID NO: 52;
   an HCDR2 comprising SEQ ID NO: 44, SEQ ID NO: 47, SEQ ID NO: 50, or SEQ ID NO: 53;
   an HCDR3 comprising SEQ ID NO: 45, SEQ ID NO: 48, SEQ ID NO: 51, or SEQ ID NO: 54;
   an LCDR1 comprising SEQ ID NO: 15, SEQ ID NO: 18, SEQ ID NO: 21, or SEQ ID NO: 24;
   an LCDR2 comprising SEQ ID NO: 16, SEQ ID NO: 19, or amino acid sequence LAS, and;
   an LCDR3 comprising SEQ ID NO: 17, SEQ ID NO: 20, SEQ ID NO: 23, or SEQ ID NO: 26.

Embodiment 187. The use according to Embodiment 186, wherein the antibody is bispecific.

Embodiment 188. The use according to Embodiment 186 or 187, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 189. The use according to any one of Embodiments 186 to 188, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 190. The use according to any one of Embodiments 186 to 188, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 191. The use according to any one of Embodiments 186 to 190, wherein the atopic dermatitis is moderate-to-severe atopic dermatitis.

Embodiment 192. The use according to any one of Embodiments 186 to 191, wherein the at least one maintenance dose is administered subcutaneously or intravenously, preferably subcutaneously.

Embodiment 193. The use according to any one of Embodiments 186 to 192, wherein the at least one maintenance dose comprises an amount of the multispecific antibody or antigen-binding fragment thereof selected from the group consisting of about 60 mg, about 150 mg, about 300 mg, and about 600 mg.

Embodiment 194. The use according to any one of Embodiments 186 to 193, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

Embodiment 195. The use according to Embodiment 194, wherein each maintenance dose comprises about 60 mg of the multispecific antibody or antigen-binding fragment thereof.

Embodiment 196. The use according to Embodiment 194, wherein each maintenance dose comprises about 150 mg of the multispecific antibody or antigen-binding fragment thereof.

Embodiment 197. The use according to Embodiment 194, wherein each maintenance dose comprises about 300 mg of the multispecific antibody or antigen-binding fragment thereof.

Embodiment 198. The use according to Embodiment 194, wherein each maintenance dose comprises about 600 mg of the multispecific antibody or antigen-binding fragment thereof.

Embodiment 199. The use according to any one of Embodiments 186 to 198, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

Embodiment 200. The use according to any one of Embodiments 186 to 199, wherein the subject has received at least three loading doses of the multispecific antibody or antigen-binding fragment thereof.

Embodiment 201. The use according to any one of Embodiments 186 to 200, wherein a maintenance dose is administered two weeks, four weeks, eight weeks, or twelve weeks after the last loading dose has been administered to the subject.

### General

The contents of any patents, patent applications, and references cited throughout this specification are hereby incorporated by reference in their entireties. Unless otherwise required by context, singular terms used herein shall include pluralities and plural terms shall include the singular.

Various embodiments of the invention are described herein. It will be recognized that features specified in each embodiment may be combined with other specified features to provide further embodiments of the present invention. When an embodiment is described as being “according to” or “of” a previous embodiment, the previous embodiment includes sub-embodiments thereof.

The present disclosure includes a sequence listing. Certain sequences represented by specific SEQ ID NOs are further included in the detailed description. In cases where a discrepancy arises between a sequence having a SEQ ID NO in the detailed description and a sequence having the same SEQ ID NO as disclosed in the sequence listing, the detailed description prevails.

The following examples are provided to further illustrate the disclosure but should not be considered to limit its scope in any way. Other variants of the disclosure will be readily apparent to one of ordinary skill in the art and are encompassed by the description and the appended claims.

### EXAMPLES

### Example 1. A First-In-Human, Randomized, Participant-Blinded, Placebo-Controlled, Single Ascending Dose Study to Investigate the Safety, Tolerability and Pharmacokinetics of an Anti-IL-18/IL-13 Bispecific Antibody in Healthy Volunteers

This first-in-human (FiH) study (Trial ID: jRCT2071230087) aimed to assess the safety, tolerability, and pharmacokinetics of an anti-IL-18/IL-13 IgG1 bispecific antibody (SEQ ID NO: 42, 14, 56, 28) in human healthy volunteers. A total of 56 participants were enrolled.

### Objectives of the Study

Objectives and related Endpoints are listed in the following Table:

TABLE 2
Objectives and related Endpoints

| Objective(s) | Endpoint(s) |
|---|---|
| Primary Objective | Endpoint(s) for primary objective |
| To assess the safety and tolerability of the anti- | Treatment emergent adverse events, vital signs, ECG |
| IL-18/IL-13 antibody after a single i.v./s.c. | data, laboratory assessments (hematology, chemistry, |
| dose in healthy adult participants | urinalysis, coagulation) |
| Secondary Objective | Endpoint(s) for secondary objective(s) |
| To assess the pharmacokinetics (PK) of the | Anti-IL-18/IL-13 antibody serum concentration-time |
| anti-IL-18/IL-13 antibody after a single | data and derived PK parameters |
| i.v./s.c. dose in healthy adult participants | |
| Exploratory objective(s) | Endpoint(s) for exploratory objective(s) |
| To inform on pharmacodynamics and pathway | Changes from baseline of phosphorylation of STAT6 |
| engagement of the anti-IL-18/IL-13 antibody | and IFNγ |
| To determine target engagement of the anti-IL- | Concentration of exploratory target capture biomarkers |
| 18/IL-13 antibody | (accumulation of IL-13, IL-18 after anti-IL-18/IL-13 |
| | antibody binding) |

Key Inclusion and Exclusion Criteria were as Follows:

### Key Inclusion Criteria

Healthy male or non-childbearing potential female participants 18 to 55 years of age, inclusive, at the time of screening, and in good health as determined by past medical history, physical examination, vital signs, electrocardiogram, and laboratory tests at screening and baseline.
Weigh at least 45.0 kg (female) and 50.0 kg (male) with a body mass index (BMI) within the range of 18.5-29.9 kg/m²

### Key Exclusion Criteria

Significant illness, including infectious diseases that has not resolved within 2 weeks prior to baseline, or positive SARS-CoV-2 test result at baseline, or contact with a known case of COVID-19 infection in the 2 weeks prior to baseline
Evidence of active or latent Tuberculosis (TB) infection, as determined by T-Spot test at screening
Use of other investigational drugs within the last 30 days or 5 half-lives of the other drugs prior to initial dosing, whichever is longer.
Use of any prescription drugs, over-the-counter (OTC) medications, herbal supplements within the last 4 weeks prior to initial dosing.
Smokers (use of tobacco/nicotine products within 3 months of dosing

### Study Design

The study was a single ascending dose (SAD) study consisting of five cohorts (A1 to A5) that received anti-IL-18/IL-13 antibody intravenously (i.v.) and two cohorts (B1 and B2) that received anti-IL-18/IL-13 antibody subcutaneously (s.c.). Eight participants were randomized per cohort to receive a single dose of the antibody or matching placebo in a 6:2 ratio. No stratification was used in the randomization. An overview of the study design is given below:

TABLE 3
F-i-H study design

| | |
|---|---|
| Cohort A1 | i.v. dose 0.3 mg/kg |
| Cohort A2 | i.v. dose 1 mg/kg |
| Cohort A3 | i.v. dose 3 mg/kg |
| Cohort A4 | i.v. dose 10 mg/kg |
| Cohort A5 | i.v. dose 30 mg/kg |
| Cohort B1 | s.c. dose 300 mg |
| Cohort B2 | s.c. dose 600 mg |

An interim analysis was conducted after cohort B2 was fully enrolled and all the enrolled participants had at least 30 days of follow-up (Day 29 visit).

The PK exposure measurements in this study were the total serum anti-IL-18/IL-13 antibody concentrations. Presented PK samples were collected on Day 1: −1h (pre-dose), 2 h, 3h (for the i.v. cohorts), 4h (for the i.v. cohorts), 6 h, 12 h; Day 2 (24h); Day 3 (48h), and on Days 5, 8, 15, 29, 43, 57, 85, 113, and 155. The lower limit of quantification (LLQQ) was defined as 0.02 μg/mL and there were no samples with values below lower limit of quantification (BLOQ) in the analyzed data set.

Samples for biomarker analysis (CCL17/TARC (Thymus and activation-regulated chemokine, IL-18, pSTAT6, IFNγ) were collected pre-dose and on day 3, day 8 (IL-18, only), day 29, and day 85 post administration. CCL17/TARC levels were measured using the MSD V-plex assay kit (Cat No K151AND) and IFN-gamma (IFNγ) levels were measured using the MSD S-PLEX assay kit (Cat No K151X9S), according to the manufacturer's protocol. IL-18 levels were measured using ELISA.

Additionally, the effect of the anti-IL-18/IL-13 bispecific antibody on downstream pathways (pSTAT6 and IFNγ expression) was assessed using ex-vivo stimulation assays performed on collected whole blood samples. Samples were stimulated either with rhIL-13 (pSTAT6) or rhIL-18 and rhIL-12p70 (IFNγ). Supernatant from rhIL-18 and rhIL-12p70 stimulated samples were used to measure IFNγ levels using ELISA. RhIL-13-stimulated cells were used to measure pSTAT6 levels on monocytes by flow cytometry.

### Results

### Interim Analysis

Results of the interim analysis are presented below.

TABLE 4
Participants with adverse effects (AEs) by preferred term, and treatment by frequency

| | i.v. | i.v. | i.v. | i.v. | i.v. | i.v. | s.c. | s.c. | s.c. | |
|---|---|---|---|---|---|---|---|---|---|---|
| | 0.3 mg/kg | 1 mg/kg | 3 mg/kg | 10 mg/kg | 30 mg/kg | placebo | 300 mg | 600 mg | placebo | Total |
| Preferred | N = 6 | N = 6 | N = 6 | N = 6 | N = 6 | N = 10 | N = 6 | N = 6 | N = 4 | N = 56 |
| term | n (%) | n (%) | n (%) | n (%) | n (%) | n (%) | n (%) | n (%) | n (%) | n (%) |
| Number of | 1 | 2 | 3 | 2 | 1 | 7 | 1 | 1 | 1 | 19 |
| participants | (16.7) | (33.3) | (50.0) | (33.3) | (16.7) | (70.0) | (16.7) | (16.7) | (25.0) | (33.9) |
| with at | | | | | | | | | | |
| least one | | | | | | | | | | |
| event | | | | | | | | | | |
| Nasophar- | 1 | 1 | 2 | 2 | 1 | 3 | 0 | 0 | 0 | 10 |
| yngitis | (16.7) | (16.7) | (33.3) | (33.3) | (16.7) | (30.0) | | | | (17.9) |
| Blood | 0 | 1 | 1 | 0 | 0 | 0 | 0 | 0 | 1 | 3 |
| Creatine | | (16.7) | (16.7) | | | | | | (25.0) | (5.4) |
| Phospho- | | | | | | | | | | |
| kinase | | | | | | | | | | |
| Increased | | | | | | | | | | |
| Amylase | 0 | 1 | 1 | 0 | 0 | 0 | 0 | 0 | 0 | 2 |
| Increased | | (16.7) | (16.7) | | | | | | | (3.6) |
| Alanine | 0 | 0 | 0 | 0 | 0 | 1 | 0 | 0 | 0 | 1 |
| Amino | | | | | | (10.0) | | | | (1.8) |
| transferase | | | | | | | | | | |
| Increased | | | | | | | | | | |
| Conjunctivitis | 0 | 0 | 0 | 0 | 0 | 0 | 0 | 1 | 0 | 1 |
| Allergic | | | | | | | | (16.7) | | (1.8) |
| Contusion | 0 | 0 | 0 | 0 | 0 | 1 | 0 | 0 | 0 | 1 |
| | | | | | | (10.0) | | | | (1.8) |
| Dental | 0 | 0 | 0 | 0 | 0 | 1 | 0 | 0 | 0 | 1 |
| Caries | | | | | | (10.0) | | | | (1.8) |
| Dizziness | 0 | 0 | 0 | 0 | 0 | 0 | 1 | 0 | 0 | 1 |
| | | | | | | | (16.7) | | | (1.8) |
| Gamma- | 0 | 0 | 0 | 0 | 0 | 1 | 0 | 0 | 0 | 1 |
| Glutamyl | | | | | | (10.0) | | | | (1.8) |
| Transferase | | | | | | | | | | |
| Increased | | | | | | | | | | |
| Lipase | 0 | 0 | 1 | 0 | 0 | 0 | 0 | 0 | 0 | 1 |
| Increased | | | (16.7) | | | | | | | (1.8) |
| Low | 0 | 0 | 0 | 0 | 0 | 1 | 0 | 0 | 0 | 1 |
| Density | | | | | | (10.0) | | | | (1.8) |
| Lipoprotein | | | | | | | | | | |
| Increased | | | | | | | | | | |
| Miliaria | 0 | 0 | 0 | 1 | 0 | 0 | 0 | 0 | 0 | 1 |
| | | | | (16.7) | | | | | | (1.8) |
| Pyrexia | 0 | 0 | 0 | 0 | 0 | 1 | 0 | 0 | 0 | 1 |
| | | | | | | (10.0) | | | | (1.8) |
| Rash | 0 | 0 | 0 | 0 | 0 | 1 | 0 | 0 | 0 | 1 |
| | | | | | | (10.0) | | | | (1.8) |
| Skin | 0 | 0 | 0 | 0 | 0 | 0 | 1 | 0 | 0 | 1 |
| Laceration | | | | | | | (16.7) | | | (1.8) |

A participant with multiple occurrences of an adverse effect was counted only once in the AE category
N = number of Participants studied;
n = % of Participants with at least one AE in the category

All AEs in the anti-IL-18/IL-13 bispecific antibody treatment arms were mild except one event of nasopharyngitis (in 1 mg/kg cohort) which was moderate. No serious or severe AEs were observed. There were no apparent imbalances in AEs between active and placebo groups. The interim safety results demonstrated that all tested doses were well-tolerated and safe in humans.

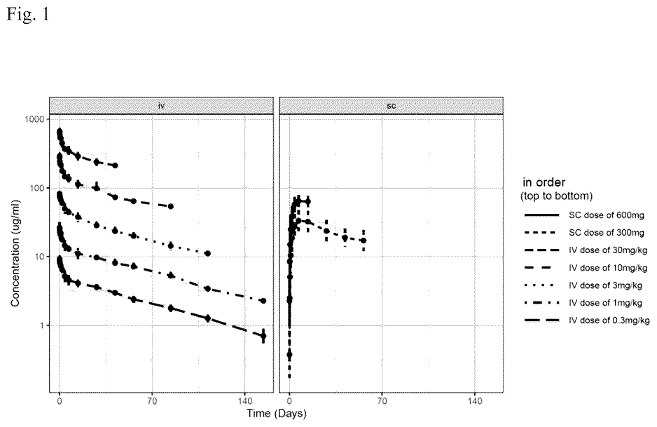

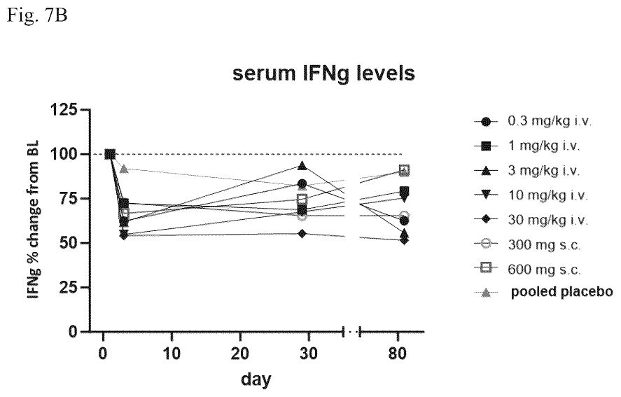

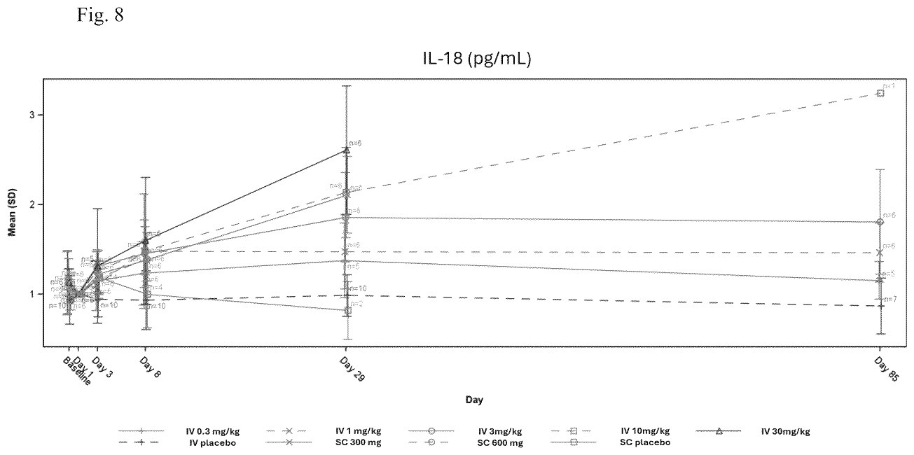

The antibody PK concentrations observed over time in this study are provided in FIG. 1. CL17/TARC, IFNγ, and IL-18 relative reduction (in %) from baseline over time is shown in FIG. 7A (CCL17/TARC), FIG. 7B (IFNγ), and FIG. 8 (IL-18).

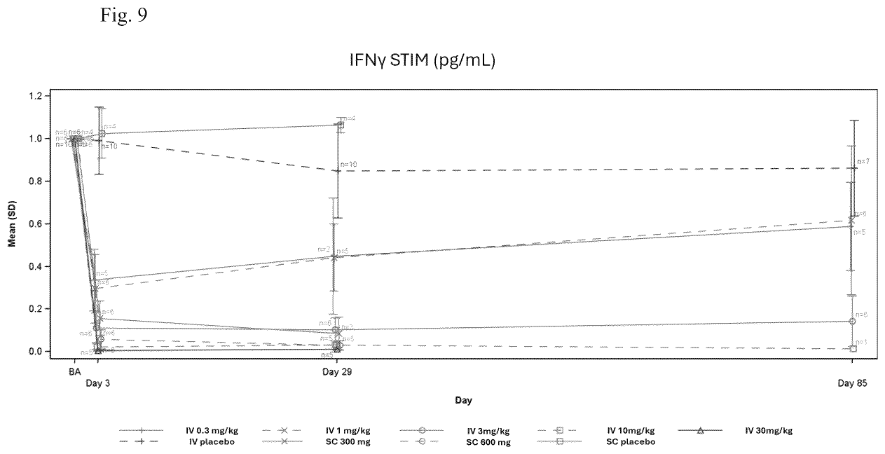

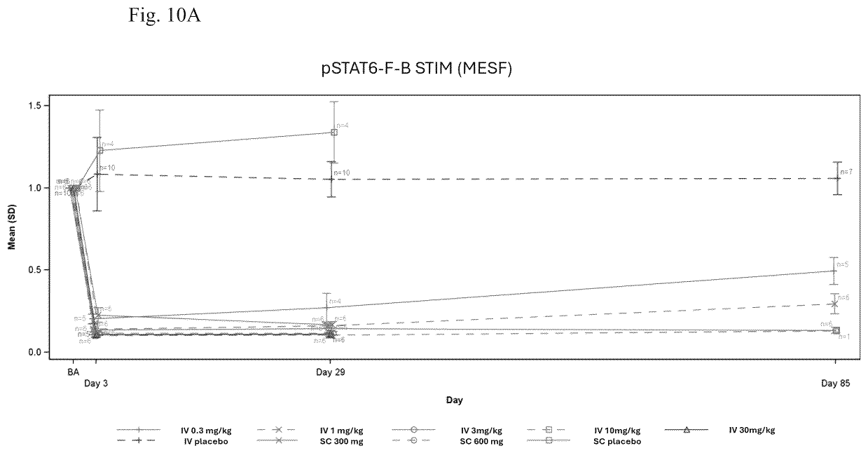

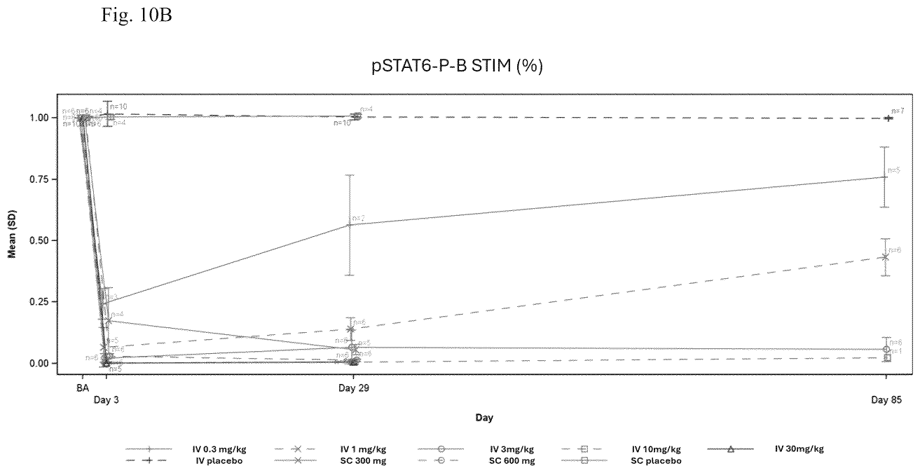

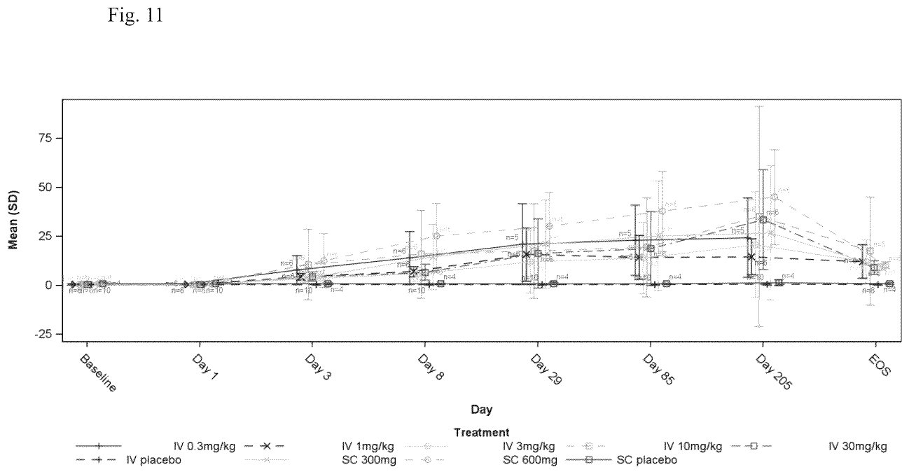

The effects of the anti-IL-18/IL-13 bispecific antibody on downstream pathways (pSTAT6 and IFNγ expression) was assessed using ex-vivo stimulation assays performed on collected samples. Supernatant from rhIL-18 and rhIL-12p70 stimulated samples were used to measure IFNγ levels using ELISA (results of mean (SD) IFNγ ratio to baseline are shown in FIG. 9). RhIL-13-stimulated cells were used to measure pSTAT6 levels on monocytes by flow cytometry (mean (SD) ratio to baseline results are shown in FIG. 10A and FIG. 10B). Assay results in the interim analysis demonstrated target engagement as well as an effect of the anti-IL-18/IL-13 antibody on downstream pathways under the tested dosages.

### Final Analysis

The safety assessment following end-of-study (EoS; Day 300) is shown below.

TABLE 5
Participants with adverse events (AEs) by preferred term and
treatment by frequency (≥2 participants in total) - EoS

| | i.v. | i.v. | i.v. | i.v. | i.v. | i.v. | s.c. | s.c. | s.c. | |
|---|---|---|---|---|---|---|---|---|---|---|
| | 0.3 mg/kg | 1 mg/kg | 3 mg/kg | 10 mg/kg | 30 mg/kg | placebo | 300 mg | 600 mg | placebo | Total |
| Preferred | N = 6 | N = 6 | N = 6 | N = 6 | N = 6 | N = 10 | N = 6 | N = 6 | N = 4 | N = 56 |
| term | n (%) | n (%) | n (%) | n (%) | n (%) | n (%) | n (%) | n (%) | n (%) | n (%) |
| Number of | 1 | 2 | 3 | 4 | 4 | 8 | 4 | 4 | 2 | 32 |
| participants | (16.7) | (33.3) | (50.0) | (66.7) | (66.7) | (80.0) | (66.7) | (66.7) | (50.0) | (57.1) |
| with at | | | | | | | | | | |
| least one | | | | | | | | | | |
| event | | | | | | | | | | |
| Blood | 0 | 1 | 1 | 1 | 0 | 0 | 1 | 1 | 1 | 6 |
| Creatine | | (16.7) | (16.7) | (16.7) | | | (16.7) | (16.7) | (25.0) | (10.7) |
| Phosphokinase | | | | | | | | | | |
| Increased | | | | | | | | | | |
| Nasophar- | 1 | 1 | 2 | 3 | 3 | 4 | 1 | 2 | 1 | 18 |
| yngitis | (16.7) | (16.7) | (33.3) | (50.0) | (50.0) | (40.0) | (16.7) | (33.3) | (25.0) | (32.1) |
| Alanine | 0 | 0 | 0 | 0 | 0 | 2 | 0 | 0 | 0 | 2 |
| Aminotrans- | | | | | | (20.0) | | | | (3.6) |
| ferase | | | | | | | | | | |
| increased | | | | | | | | | | |
| Amylase | 0 | 1 | 1 | 0 | 0 | 0 | 0 | 0 | 0 | 2 |
| Increased | | (16.7) | (16.7) | | | | | | | (3.6) |
| Covid-19 | 0 | 0 | 1 | 0 | 0 | 0 | 1 | 0 | 0 | 2 |
| | | | (16.7) | | | | (16.7) | | | (3.6) |
| Dental | 0 | 0 | 0 | 0 | 0 | 1 | 0 | 0 | 1 | 2 |
| Caries | | | | | | (10.0) | | | (25.0) | (3.6) |
| Lipase | 0 | 0 | 1 | 0 | 1 | 0 | 0 | 0 | 0 | 2 |
| Increased | | | (16.7) | | (16.7) | | | | | (3.6) |
| Pyrexia | 0 | 0 | 0 | 0 | 1 | 1 | 0 | 0 | 0 | 2 |
| | | | | | (16.7) | (10.0) | | | | (3.6) |

A participant with multiple occurrences of an adverse effect was counted only once in the AE category
N = number of Participants studied;
n = % of Participants with at least one AE in the category;
Only adverse events occurring at or after start of infusion/injection were included

TABLE 6
Participants with adverse events by system organ class and treatment by frequency - EoS

| | i.v. | i.v. | i.v. | i.v. | i.v. | i.v. | s.c. | s.c. | s.c. | |
|---|---|---|---|---|---|---|---|---|---|---|
| | 0.3 mg/kg | 1 mg/kg | 3 mg/kg | 10 mg/kg | 30 mg/kg | placebo | 300 mg | 600 mg | placebo | Total |
| Preferred | N = 6 | N = 6 | N = 6 | N = 6 | N = 6 | N = 10 | N = 6 | N = 6 | N = 4 | N = 56 |
| term | n (%) | n (%) | n (%) | n (%) | n (%) | n (%) | n (%) | n (%) | n (%) | n (%) |
| Number of | 1 | 2 | 3 | 4 | 4 | 8 | 4 | 4 | 2 | 32 |
| participants | (16.7) | (33.3) | (50.0) | (66.7) | (66.7) | (80.0) | (66.7) | (66.7) | (50.0) | (57.1) |
| with at | | | | | | | | | | |
| least one | | | | | | | | | | |
| event | | | | | | | | | | |
| Eye | 0 | 0 | 0 | 0 | 0 | 0 | 0 | 1 | 0 | 1 |
| Disorders | | | | | | | | (16.7) | | (1.8) |
| Gastro- | 0 | 0 | 0 | 0 | 0 | 1 | 0 | 0 | 1 | 2 |
| intestinal | | | | | | (10.0) | | | (25.0) | (3.6) |
| Disorders | | | | | | | | | | |
| General | 0 | 0 | 0 | 0 | 1 | 1 | 0 | 0 | 0 | 2 |
| Disorders | | | | | (16.7) | (10.0) | | | | (3.6) |
| and | | | | | | | | | | |
| Administration | | | | | | | | | | |
| Site | | | | | | | | | | |
| Conditions | | | | | | | | | | |
| Hepatobiliary | 0 | 0 | 0 | 0 | 1 | 0 | 0 | 0 | 0 | 1 |
| Disorders | | | | | (16.7) | | | | | (1.8) |
| Immune | 0 | 0 | 0 | 0 | 0 | 0 | 0 | 1 | 0 | 1 |
| System | | | | | | | | (16.7) | | (1.8) |
| Disorders | | | | | | | | | | |
| Infections | 1 | 1 | 2 | 3 | 3 | 4 | 2 | 2 | 1 | 19 |
| and | (16.7) | (16.7) | (33.3) | (50.0) | (50.0) | (40.0) | (33.3) | (33.3) | (25.0) | (33.9) |
| Infestations | | | | | | | | | | |
| Injury, | 0 | 0 | 0 | 0 | 0 | 1 | 1 | 0 | 0 | 2 |
| Poisoning | | | | | | (10.0) | (16.7) | | | (3.6) |
| and | | | | | | | | | | |
| Procedural | | | | | | | | | | |
| Complications | | | | | | | | | | |
| Investigations | 0 | 2 | 2 | 1 | 2 | 2 | 2 | 1 | 1 | 13 |
| | | (33.3) | (33.3) | (16.7) | (33.3) | (20.0) | (33.3) | (16.7) | (25.0) | (23.2) |
| Metabolism | 0 | 0 | 0 | 0 | 1 | 0 | 1 | 0 | 0 | 2 |
| and | | | | | (16.7) | | (16.7) | | | (3.6) |
| Nutrition | | | | | | | | | | |
| Disorders | | | | | | | | | | |
| Musculo- | 0 | 0 | 0 | 0 | 0 | 1 | 0 | 0 | 1 | 2 |
| skeletal and | | | | | | (10.0) | | | (25.0) | (3.6) |
| Connective | | | | | | | | | | |
| Tissue | | | | | | | | | | |
| Disorders | | | | | | | | | | |
| Nervous | 0 | 0 | 0 | 1 | 0 | 0 | 1 | 0 | 0 | 2 |
| System | | | | (16.7) | | | (16.7) | | | (3.6) |
| Disorders | | | | | | | | | | |
| Renal and | 0 | 0 | 0 | 0 | 1 | 0 | 0 | 0 | 0 | 1 |
| Urinary | | | | | (16.7) | | | | | (1.8) |
| Disorders | | | | | | | | | | |
| Skin and | 0 | 0 | 0 | 1 | 0 | 1 | 0 | 1 | 0 | 3 |
| Subcutaneous | | | | (16.7) | | (10.0) | | (16.7) | | (5.4) |
| Tissue | | | | | | | | | | |
| Disorders | | | | | | | | | | |

A participant with multiple occurrences of an adverse effect was counted only once in the AE category
N = number of Participants studied;
n = % of Participants with at least one AE in the category;
Only adverse events occurring at or after start of infusion/injection were included

TABLE 7
Overall incidence of AEs - number of events and number of participants - EoS

| | i.v. | i.v. | i.v. | i.v. | i.v. | i.v. | s.c. | s.c. | s.c. | |
|---|---|---|---|---|---|---|---|---|---|---|
| | 0.3 mg/kg | 1 mg/kg | 3 mg/kg | 10 mg/kg | 30 mg/kg | placebo | 300 mg | 600 mg | placebo | Total |
| | N = 6 | N = 6 | N = 6 | N = 6 | N = 6 | N = 10 | N = 6 | N = 6 | N = 4 | N = 56 |
| | nE, nS | nE, nS | nE, nS | nE, nS | nE, nS | nE, nS | nE, nS | nE, nS | nE, nS | nE, nS |
| | (%) | (%) | (%) | (%) | (%) | (%) | (%) | (%) | (%) | (%) |
| AEs, | 1, 1 | 4, 2 | 6, 3 | 7, 4 | 13, 4 | 15, 8 | 8, 4 | 11, 4 | 4, 2 | 69, 32 |
| participant | (16.7) | (33.3) | (50.0) | (66.7) | (66.7) | (80.0) | (66.7) | (66.7) | (50.0) | (57.1) |
| with AEs | | | | | | | | | | |
| AEs of | 1, 1 | 3, 2 | 6.3 | 7, 4 | 6, 4 | 14, 8 | 8, 4 | 9, 3 | 4, 2 | 58, 31 |
| mild | (16.7) | (33.3) | (50.0) | (66.7) | (66.7) | (80.0) | (66.7) | (50.0) | (50.0) | (55.4) |
| intensity | | | | | | | | | | |
| AEs of | 0 | 1, 1 | 0 | 0 | 0 | 1, 1 | 0 | 2, 2 | 0 | 4, 4 |
| moderate | | (16.7) | | | | (10.0) | | (33.3) | | (7.1) |
| intensity | | | | | | | | | | |
| AEs of | 0 | 0 | 0 | 0 | 7, 1 | 0 | 0 | 0 | 0 | 7, 1 |
| severe | | | | | (16.7) | | | | | (1.8) |
| intensity | | | | | | | | | | |
| Study | 0 | 0 | 0 | 0 | 0 | 0 | 0 | 0 | 0 | 0 |
| drug- | | | | | | | | | | |
| related | | | | | | | | | | |
| AEs | | | | | | | | | | |
| Serious | 0 | 0 | 0 | 0 | 3, 1 | 0 | 0 | 0 | 0 | 3, 1 |
| AEs | | | | | (16.7) | | | | | (1.8) |

N = number of Participants studied;
nE = number of AE events in the category;
nS = number of participants with at least one AE in the category;
% is based on the number of participants

When considering the entire study period (up to EoS), 32 participants (57.1%) experienced at least one AE (Table 7). The majority of the participants experienced AEs of mild intensity (55.4%). Overall, the most frequently reported AEs during the study period were infections and infestations (33.9%), investigations (23.2%) and skin and subcutaneous tissue disorders (5.4%), with no increased frequency in antibody-dosing groups compared to placebo (Table 6). Overall, the most frequently (≥5%) reported AEs during the study period were nasopharyngitis (32.1%) and blood creatine phosphokinase increased (10.7%) (Table 5).

One participant who received 30 mg/kg i.v. of the antibody experienced an episode of pyelonephritis (clinical symptoms started 205 days after dosing) which led to septic shock and acute kidney injury (Table 7). In the course of these SAEs, increased amylase levels and intermittent elevation of liver function parameters were observed. The participant did fully recover from all AEs during the observation phase of the study. No deaths were reported. Majority of the other reported AEs were common findings in the general healthy study participant population, and there was no clear difference observed in frequency of AEs between the antibody and placebo cohorts.

Overall, none of the reported AEs were suspected of being treatment-related by the treating physician. No clinically significant clinical laboratory, vital signs and ECG abnormalities were reported, and no clinically significant trends were observed across the tested parameters. Overall, the final analysis confirmed the interim analysis in that the anti-IL-18/IL-13 bispecific antibody was well tolerated across all the dose levels evaluated in this study.

The final analysis included biomarker analysis samples collected on day 205 post-dose and on EOS (Day 300), measured as described above. Additionally, IL-13 levels were measured using ELISA.

As shown in FIG. 11, following i.v. and s.c. administration of the anti-IL-18/IL-13 bispecific antibody, total IL-13 serum concentration increased upon antibody exposure, whilst no change was observed after placebo administration. The peak mean total IL-13 concentration was observed on Day 205 across all dosing cohorts, except for 1 mg/kg dose, where peak mean concentration was observed on Day 29. A decline in total IL-13 serum concentration was observed across all doses by EoS (Day 300), reaching near baseline values. A high variability (SD) in total IL-13 serum concentration was observed among the participants for both i.v. and s.c. cohorts. The increased serum concentration of total IL-13 after antibody administration is indicative of drug-target complex formation leading to slower elimination, demonstrating the binding of the antibody to IL-13.

IFNγ inhibition, measured as mean percentage change from baseline (FIG. 12), was observed across all dosing cohorts. Following the antibody intravenous administration a substantial IFNγ inhibition was observed for dose ≤1.0 mg/kg (66.3% to 70.5%), and near complete IFNγ inhibition was observed for doses ≥3 mg/kg on Day 3 (88.7% to 99.4%). A near complete IFNγ inhibition lasted until Day 205 in the 10 mg/kg (90.2%) and 30 mg/kg (92.3%) dose groups. Following antibody subcutaneous administration a near complete IFNγ inhibition (88.0% in the 300 mg dose group and 93.9% in the 600 mg group) was observed for both doses lasting until Day 85 (FIG. 12).

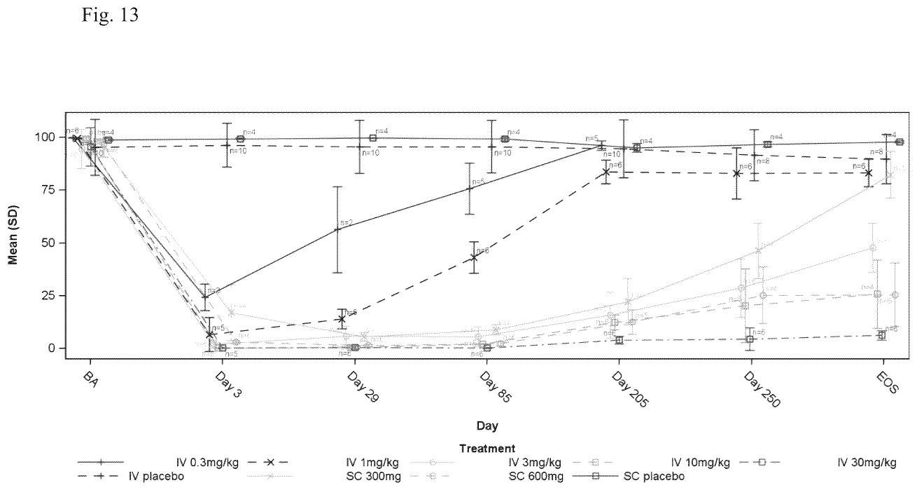

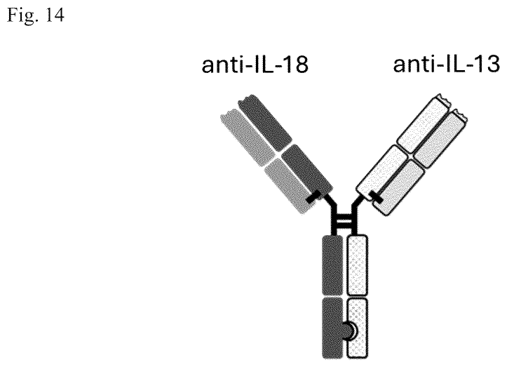

Following intravenous administration, a rapid reduction of the percentage of pSTAT6-positive blood monocytes was observed across all cohorts. The lowest percentage of pSTAT6-positive monocytes was observed on Day 3 across all i.v. cohorts (24.2% in 0.3 mg/kg, 6.6% in 1 mg/kg, 2.2% in 3 mg/kg, 0.2% in 10 mg/kg and 30 mg/kg cohorts) (FIG. 13). After Day 3, the levels of pSTAT6-positive monocytes gradually increased in a dose dependent manner, more noticeably at lower doses (doses ≥3 mg/kg); however, it remained well below the baseline levels, except for doses ≤1 mg/kg, where it reached near baseline levels from Day 205 onwards. Following the subcutaneous administration, reduction of the percentage of pSTAT6-positive blood monocytes was observed across both doses. The lowest percentage of pSTAT6-positive monocytes was observed on Day 29 (5.6% in 300 mg s.c. and 1.3% in 600 mg s.c. cohorts). After Day 29, levels of pSTAT6-positive monocytes gradually increased and returned near to baseline for dose 300 mg on Day 300, while they remained well below the baseline levels for s.c. 600 mg dose. For both i.v. and s.c. cohorts the extent and duration of pSTAT6 inhibition was more pronounced in higher doses. The percentage of pSTAT6-positive monocytes remained largely stable for both the i.v. and s.c. placebo cohorts (FIG. 13).

Overall, the full analysis confirmed the interim analysis results on demonstrated target engagement as well as an effect of the anti-IL-18/IL-13 antibody on downstream pathways under the tested dosages.

### Example 2. Modelling and Simulation Supporting Anti-IL-18/IL-13 Bispecific Antibody Dosage Regimens

### Modelling Set-Up

In this Example a population PK (PopPK) model was developed and applied to propose innovative dosing regimens for the anti-IL-18/IL-13 bispecific antibody of Example 1 for patients with moderate-to-severe atopic dermatitis. The anti-IL-18/IL-13 bispecific antibody PopPK model was built using human PK data (total serum anti-IL-18/IL-13 bispecific antibody concentrations) obtained from the FiH study (Trial ID: jRCT2071230087, see Example 1). Bispecific antibody concentration-time profiles were described by a two-compartmental model, with first order-absorption to reflect subcutaneous administration, and first order elimination. Inter-individual variability was included on all model parameters. No correlation of random effects was included. Residual unexplained variability was described using a combined error model. Body weight effect was accounted for through allometric scaling with fixed exponent of 0.75 on apparent clearance and 1 on apparent volumes of distribution (central and peripheral). Model-validation was performed using standard assessment methods (goodness-of-fit and predictive checks). Bioavailability of subcutaneous administered anti-IL-18/IL-13 bispecific antibody was estimated at approximately 65% compared to intravenous administration supporting subcutaneous administration in a Phase II study in patients with moderate-to-severe atopic dermatitis. The anti-IL-18/IL-13 bispecific antibody exhibited slow clearance of 0.06 L/day. Terminal half-life was estimated at 57.1 [52.3-63.0] days (median and 95% CI) visibly exceeding half-life of a typical IgG antibody. The developed PopPK model and its final parameter estimates were used in simulations.

PK-exposure matching was applied to select the optimal dosing regimens for testing in a Phase II study in patients with moderate-to-severe AD, aiming at a higher efficacy in this patient population compared to a marketed anti-IL-13 antibody (lebrikizumab) and an anti-IL-18 antibody previously tested in a Phase II AD study (Trial ID: NCT04836858). Specifically, anti-IL-18/IL-13 bispecific antibody human PK exposures at steady-state were modelled to match or exceed PK steady-state exposures of the anti-IL-13 antibody dose regimens approved for treatment of patients with moderate-to-severe AD (250 mg Q2W and 250 mg Q4W) and of the anti-IL-18 antibody dose regimens tested in the Phase II study in patients with moderate-to-severe AD (300 mg s.c. Q4W and 10 mg/kg i.v. Q4W). PopPK models for the anti-IL-13 antibody and anti-IL-18 antibody had equivalent structure to anti-IL-18/IL-13 bispecific antibody PopPK model. Anti-IL-18 antibody PopPK model was estimated on data from a FiH study and subsequently validated with data from its Phase II study (Trial ID: NCT04836858). Parameter values of anti-IL-13 antibody PopPK model were extracted from a published model based on data from both healthy volunteers as well as asthma and atopic dermatitis patients (described in Zhu, et al., 2017, Pulm Pharmacol Ther.; 46:88-98), with exception of the body weight coefficients which were fixed to the same values as in the anti-IL-18 antibody and anti-IL-18/IL-13 bispecific antibody PopPK models.

All simulations were set up to predict PK exposures for an average individual of 70 kg accounting for uncertainty in parameter estimates (based on relative standard error of parameter estimates [RSE]) without inclusion of inter-individual variability. Summary statistics including population median, and its uncertainty measured through 95% prediction interval (PI) were derived from 1000 simulated individuals (each with simulated different individual PK parameter estimates). To adjust for stoichiometric differences, the PK exposures of the anti-IL-13 and the anti-IL-18 antibodies were multiplied by a factor of 2 (i.e., “equivalent” PK exposures).

### Results of Modelling Study

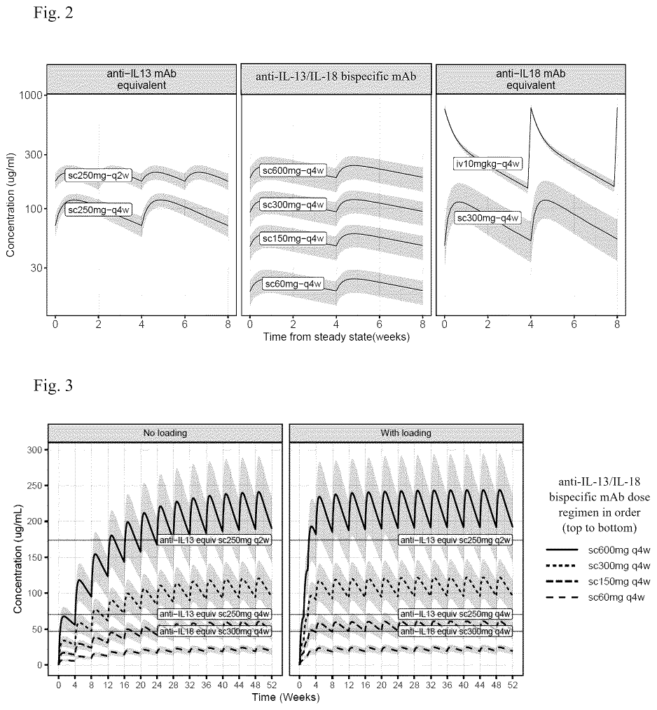

Application of the PopPK model enabled to predict PK exposures after multiple dose administration (and at steady-state) for a number of different dosing regimens, including: 60 mg Q4W (once every 4 weeks) subcutaneously (s.c.), 150 mg Q4W s.c., 300 mg Q4W s.c., and 600 mg Q4W s.c., and to compare them with “equivalent” (adjusted for stoichiometric differences) PK exposures of the anti-IL-13 and the anti-IL-18 antibodies, with results shown in FIG. 2. Performed simulations indicate that steady-state PK exposures for anti-IL-18/IL-13 bispecific antibody at the 300 mg Q4W and 600 mg Q4W regimens match or exceed PK exposures of the anti-IL-13 antibody (250 mg Q4W) and the anti-IL-18 antibody (300 mg Q4W). Based on the PK-exposure matching approach those treatment regimens are proposed for the treatment period from week 4 to week 16 to be studied in the planned Phase II study (see Example 3). In addition, two lower dose regimens presented in FIG. 2 will be tested (60 mg Q4W and 150 mg Q4W).

To enable patients to experience early treatment benefit a loading schedule comprising 3 doses (at day 1, week 1, and week 2) is being proposed, which was predicted to bring PK exposures to optimal range (approximate steady-state) fast and without exceeding the steady-state PK exposures level. Simulations indicated that, without loading doses, patients are expected to reach steady-state PK exposures only at ca. week 32 of treatment. Conversely, inclusion of a loading schedule comprising 3 loading doses (at day 1, week 1, and week 2) decreases the time needed to reach approximately steady-state exposures to week 4 of treatment (FIG. 3) for all dosing regimens. The simulated loading doses (at day 1, week 1, and week 2) presented to be tested in the planned Phase II study (see Example 3) are as follows: 60 mg s.c. in the case of the 60 mg Q4W s.c. dosing regimen, 150 mg s.c. in the case of the 150 mg Q4W s.c. dosing regimen, 300 mg s.c. in the case of the 300 mg Q4W s.c. dosing regimen, and 600 mg s.c. in the case of the 600 mg Q4W s.c. dosing regimen.

Further simulations were conducted to explore the possibility of decreasing dosing frequency after the first 16 weeks of treatment, which could e.g., be optimal for patients who achieve a positive response to treatment during the first 16 weeks (which comprises 3 loading doses at day 1, week 1, and week 2, followed by maintenance doses administered from week 4 onwards). The two highest anti-IL-18/IL-13 bispecific antibody doses (300 mg s.c. and 600 mg s.c.) were used as case studies. Two design options were considered: lengthening of the dosing interval to Q8W (once every 8 weeks) or to Q12W (once every 12 weeks). Results indicate that therapeutically efficacious serum levels could be maintained under both intervals (FIGS. 4 and 5).

### Example 3. A Multicenter, Randomized, Double-Blind, Placebo-Controlled, Phase II Study to Assess the Efficacy and Safety of Anti-IL-18/IL-13 Bispecific Antibody in Patients with Moderate-to-Severe Atopic Dermatitis

To assess the efficacy and safety of the anti-IL-18/IL-13 bispecific antibody (see Examples 1 and 2) in patients with moderate to severe atopic dermatitis (AD), a randomized, subject and investigator blinded, placebo-controlled multicenter study is conducted.

### Objectives of the Study

Objectives and related Endpoints are listed in Table 8:

TABLE 8
Objectives and related Endpoints

| Objective(s) | Endpoint(s) |
|---|---|
| Primary Objective | Endpoint(s) for primary objective |
| To establish and characterize the dose-response | Percentage change from baseline in the EASI score at |
| relationship of respective interventions and | Week 16 |
| estimate the targeted dose(s) treatment effect | |
| vs placebo with respect to the percentage | |
| change from baseline on EASI score at Week | |
| 16 in participants with moderate-to-severe AD | |
| Secondary Objective(s) | Endpoint(s) for secondary objective(s) |
| To establish and characterize the dose- | IGA response at Week 16: IGA score of 0 (clear) or 1 |
| response relationship of respective | (almost clear) and at least 2 points reduction from |
| interventions and estimate the targeted dose(s) | baseline IGA response at Week 16 |
| treatment effect vs placebo with respect to | EASI-75 response at week 16: ≥75% reduction |
| achievement of IGA response and EASI-75 | (improvement) from baseline in EASI score response |
| response at Week 16 in participants with | at Week 16 |
| moderate-to-severe AD | |
| To evaluate the safety and tolerability of | Treatment emergent adverse events, vital signs, ECG, |
| respective interventions/presence of any | and laboratory assessments (incl. physical |
| adverse effects | examination, hematology, chemistry, urinalysis, |
| | immunogenicity) |

### Primary Estimand

The primary estimand is described by the following attributes:
Population: Participants having moderate-to-severe AD for at least 1 year, baseline EASI score ≥16, baseline IGA score ≥3, baseline BSA ≥10% and meeting study inclusion/exclusion criteria
Endpoint: Percentage change from baseline in the EASI score at Week 16
Treatment of interests: The investigational treatment of active dose(s) vs. placebo control
Summary measure: Difference in mean percent change from baseline in EASI score at Week 16 between active treatment group and control group

### Secondary Estimand

The secondary estimand is described by the following attributes (population and treatment of interest is the same as for the primary estimand):
Endpoint: IGA response at Week 16: IGA score of 0 (clear) or 1 (almost clear) and at least 2 points reduction from baseline IGA scale at Week 16; EASI-75 response at Week 16: ≥75% reduction (improvement) from baseline in EASI score response at Week 16
Summary measure: Difference in proportion of response between treatment groups (each active treatment group vs. placebo)

### Key Inclusion Criteria

Patients with a diagnosis of AD and onset of disease for at least 1 year
Moderate-to-severe AD as defined by all of the following:
   EASI score ≥12 at screening and ≥16 at baseline
   IGA score ≥3 at screening and baseline
   Body surface area (BSA) ≥10% at screening and baseline
History of inadequate response to treatment with topical medications or for whom topical treatments are medically inadvisable as per Investigator judgement

### Key Exclusion Criteria

Participants with a clinically significant medical condition (e.g., active tuberculosis, acute or chronic infection with hepatitis B or C, history of HIV infection, known or suspected history of immunosuppression or immune deficiency)
Participants with active chronic or acute infection requiring treatment with systemic therapy within 14 days before the baseline visit
Participants with clinically significant abnormal hematology, biochemistry, or urine test results or/and clinically significant abnormal ECG
Participants with any other active inflammatory skin disease.
Participants with any chronic, uncontrolled medical condition, which would put the participant at increased risk during study (e.g., uncontrolled: diabetes, hypertension)
Participants with any clinically unstable disease states that would likely require systemic corticosteroids (e.g., asthma).
Participants with a history of anaphylaxis due to any biologic therapy

### Study Design

### Eczema Area and Severity Index (EASI)

The EASI will be used to assess the extent and severity of AD (Hanifin et al. 2022, Dermatitis 33 (3): 187-92). The Investigator or trained qualified designee will complete the EASI assessment on each of the visits as outlined in the assessment schedule. Whenever possible, the EASI assessments should be performed by the same evaluator throughout the study. Each body region (head/neck [H], upper extremities [UE], trunk [T], and lower extremities [LE]) will be assessed for:
Severity of AD: the average degree of the following key signs of AD (erythema, induration/papulation, excoriation, and lichenification) will each be assigned a score of 0, 1, 2 or 3 indicating none (0), mild (1), moderate (2), and severe (3) expression of the clinical sign.
Extent of AD: Based on the extent of AD in a particular body region (when each body region is considered as a whole or 100%), an Area score will be assigned to that body region.

### Notes

Only inflamed areas should be included in the assessment; dry skin or post inflammatory pigmentation changes should not be included.
The neck is assessed as part of the head region.
The axillae and groin are assessed as part of the trunk.
The buttocks and feet are assessed as part of the lower limbs.

### Calculation of BSA (Body Surface Area Affected by AD)

Percentage of each body region affected by AD will be multiplied by its respective body region corresponding factor (0.1 for head, 0.2 for upper limbs, 0.3 for trunk, and 0.4 for lower limbs). Total BSA affected by AD=(0.1×Head area %)+(0.2×Upper limbs area %)+(0.3×Trunk area 10%)+(0.4×Lower limbs area %).

EASI severity descriptions are provided in the following Table:

TABLE 9
EASI severity descriptions.

| | | |
|---|---|---|
| Sign | | |
| score | Grading | Description |

Erythema (E)

| | | |
|---|---|---|
| 0 | None | N/A |
| 1 | Mild | Faintly detectable erythema: very light pink |
| 2 | Moderate | Dull red, clearly distinguishable |
| 3 | Severe | Deep/dark red |

Induration/Papulation (P)

| | | |
|---|---|---|
| 0 | None | N/A |
| 1 | Mild | Barely perceptible elevation |
| 2 | Moderate | Clearly perceptible elevation but not extensive |
| 3 | Severe | Marked and extensive elevation |

Excoriations (Ex)

| | | |
|---|---|---|
| 0 | None | N/A |
| 1 | Mild | Scant evidence of excoriations with no signs of |
| | | deeper skin damage (erosion, crust) |
| 2 | Moderate | Several linear marks of skin with some showing |
| | | evidence of deeper skin injury (erosion, crust) |
| 3 | Severe | Many erosive or crusty lesions |

Lichenification (L)

| | | |
|---|---|---|
| 0 | None | N/A |
| 1 | Mild | Slight thickening of the skin discernible only |
| | | by touch and with skin markings minimally |
| | | exaggerated |
| 2 | Moderate | Definite thickening of the skin with skin markings |
| | | exaggerated so that they form a visible |
| | | crisscross pattern |
| 3 | Severe | Thickened indurated skin with skin markings visibly |
| | | portraying an exaggerated crisscross pattern |

EASI calculation is provided in the following Table:

TABLE 10
EASI calculation.

| Body | Erythema | Induration/ | Excoriation | Licheni- | | EASI |
|---|---|---|---|---|---|---|
| region | (E) | Papulation(P) | (Ex) | fication (L) | Area score (A) | calculation |
| Head & | 0 = none | 0 = none | 0 = none | 0 = none | 0 = 0% | (E + P + Ex + |
| neck | 1 = mild | 1 = mild | 1 = mild | 1 = mild | 1 = >0-<10% | L) × A × 0.1 |
| (H) | 2 = moderate | 2 = moderate | 2 = moderate | 2 = moderate | 2 = 10% to <30% | |
| | 3 = severe | 3 = severe | 3 = severe | 3 = severe | 3 = 30% to <50% | |
| | | | | | 4 = 50% to <70% | |
| | | | | | 5 = 70% to <90% | |
| | | | | | 6 = 90% to 100% | |
| Trunk | 0-none | 0 = none | 0 = none | 0 = none | 0 = 0% | (E + P + Ex + |
| (T) | 1 = mild | 1 = mild | 1 = mild | 1 = mild | 1 = >0-<10% | L) × A × 0.3 |
| | 2 = moderate | 2 = moderate | 2 = moderate | 2 = moderate | 2 = 10% to <30% | |
| | 3 = severe | 3 = severe | 3 = severe | 3 = severe | 3 = 30% to <50% | |
| | | | | | 4 = 50% to <70% | |
| | | | | | 5 = 70% to <90% | |
| | | | | | 6 = 90% to 100% | |
| Upper | 0 = none | 0 = none | 0 = none | 0 = none | 0 = 0% | (E + P + Ex + |
| limbs | 1 = mild | 1 = mild | 1 = mild | 1 = mild | 1 = >0-<10% | L) × A × 0.2 |
| (UL) | 2 = moderate | 2 = moderate | 2 = moderate | 2 = moderate | 2 = 10% to <30% | |
| | 3 = severe | 3 = severe | 3 = severe | 3 = severe | 3 = 30% to <50% | |
| | | | | | 4 = 50% to <70% | |
| | | | | | 5 = 70% to <90% | |
| | | | | | 6 = 90% to 100% | |
| Lower | 0 = none | 0 = none | 0 = none | 0 = none | 0 = 0% | (E + P + Ex + |
| limbs | 1 = mild | 1 = mild | 1 = mild | 1 = mild | 1 = >0-<10% | L) × A × 0.4 |
| (LL) | 2 = moderate | 2 = moderate | 2 = moderate | 2 = moderate | 2 = 10% to <30% | |
| | 3 = severe | 3 = severe | 3 = severe | 3 = severe | 3 = 30% to <50% | |
| | | | | | 4 = 50% to <70% | |
| | | | | | 5 = 70% to <90% | |
| | | | | | 6 = 90% to 100% | |

| | |
|---|---|
| EASI score = | sum of the |
| | above 4 |
| | scores |

### Investigator Global Assessment (IGA)

The IGA scale used in the study is vIGA-AD™ (Validated Investigator Global Assessment scale for Atopic Dermatitis). The IGA rating scale is used to determine the severity of AD symptoms and clinical response to treatment. It reflects a participant's overall disease severity for the whole body based on a 5-point scale. The 5-point scale includes: clear, almost clear, mild, moderate, and severe disease. IGA response is defined as clear or almost clear score with at least a 2 point-reduction from baseline.

The scale is a static scale and does not refer to previous status of the participant. The Investigator or trained qualified designee will complete the IGA assessment on each of the visits. Whenever possible, the IGA assessments should be performed by the same evaluator throughout the study. The IGA should always be conducted as the first clinical assessment, before the EASI assessment. It must be based on a whole-body skin inspection.

IGA description is provided in the following Table:

TABLE 11
Investigator Global Assessment (IGA).

| Score | Morphological Description |
|---|---|
| 0-Clear | No inflammatory signs of atopic dermatitis (no erythema, no induration/papulation, no |
| | lichenification, no oozing/crusting). Post-inflammatory hyperpigmentation and/or |
| | hypopigmentation may be present. |
| 1-Almost | Barely perceptible erythema, barely perceptible induration/papulation, and/or minimal |
| Clear | lichenification. No oozing or crusting. |
| 2-Mild | Slight but definite erythema (pink), slight but definite induration/papulation, and/or |
| | slight but definite lichenification. No oozing or crusting. |
| 3-Moderate | Clearly perceptible erythema (dull red), clearly perceptible induration/papulation, |
| | and/or clearly perceptible lichenification. Oozing and crusting may be present. |
| 4-Severe | Marked erythema (deep and bright red), marked induration/papulation, and/or marked |
| | lichenification. Disease is widespread in extent. Oozing or crusting may be present. |

### Dermatology Life Quality Index (DLQI)

The Dermatology Life Quality Index (DLQI) is a 10-item general dermatology disability index designed to assess health-related quality of life in participants with eczema. The measure includes domains of daily activities, leisure, personal relationships, symptoms and feelings, treatment, and work/school. Each item has four response categories ranging from 0 (not at all) to 3 (very much). “Not relevant” is also a valid response and is scored as 0. The DLQI total score is a sum of 10 questions. Scores range from 0 to 30, with higher scores indicating greater quality of life impairment. Each subscale of the DLQI may also be analyzed separately. The overall DLQI score range is split into score bands (Hongbo et al. 2005, J Invest Dermatol 125 (4): 659-64) and validated in terms of their meaning/relevance to patients as shown in the following Table:

TABLE 12
DLQI score bands and impact on patient's life

| Score (band) | Morphological Description |
|---|---|
| 0-1 | No effect on patient's life |
| 2-5 | Small effect on patient's life |
| 6-10 | Moderate effect on patient's life |
| 11-20 | Very large effect on patient's life |
| 21-30 | Extremely large effect on patient's life |

### Peak Pruritus Numerical Rating Scale (PP-NRS)

The Peak Pruritus NRS (PP-NRS) is a well-defined measure (Yosipovitch et al. 2019, Br J Dermatol 181 (4): 761-9) to evaluate patent-reported intensity of worst itch in the previous 24 hours. Patients rate their itch on a 11-point numerical rating scale (NRS) from minimum 0 (no itch) to maximum 10 (worst itch imaginable).

### Protocol

Approximately 244 eligible adult participants (18 years and older) who have been diagnosed with moderate-to-severe AD for at least 1 year, defined by an EASI score ≥16 at baseline, an IGA score ≥3 at baseline, and BSA ≥10% at baseline, and inadequately controlled on topical therapy will be enrolled.

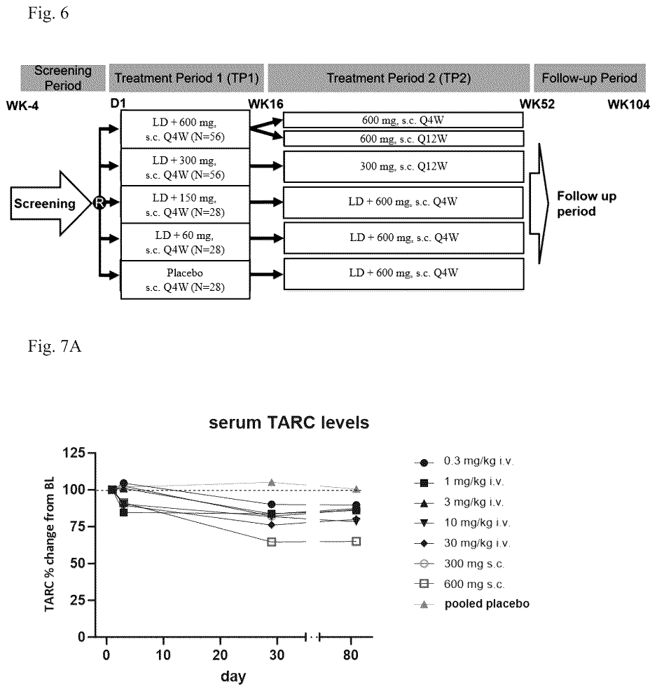

The protocol is a Phase IIb randomized, double-blind, placebo-controlled, parallel-group study with a 52-week treatment duration to assess the efficacy and safety of the anti-IL-18/IL-13 antibody for participants with moderate-to-severe AD during a 16-week placebo-controlled period (Treatment Period 1), followed by a 36-week long term treatment period (Treatment Period 2). Fifty-two (52) weeks of follow-up period is scheduled after completion/discontinuation of treatment period (FIG. 6).

Specifically, the protocol comprises the following periods (following a screening period of up to 4 weeks during which the participants are assessed for eligibility):

### Treatment Period 1 (Double-Blind, Placebo-Controlled, 16 Weeks)

Approximately 224 participants whose eligibility is confirmed at the end of the Screening period will be randomly assigned in a 2:2:1:1:2 ratio to one of the following arms (Randomization will be stratified by Baseline IGA (3 moderate versus 4 severe AD):
600 mg s.c. loading dose on Day 1, Week 1, and Week 2, followed by Q4W starting at Week 4
300 mg s.c. loading dose on Day 1, Week 1, and Week 2, followed by Q4W starting at Week 4
150 mg s.c. loading dose on Day 1, Week 1, and Week 2, followed by Q4W starting at Week 4
60 mg s.c. loading dose on Day 1, Week 1, and Week 2, followed by Q4W starting at Week 4
Placebo s.c. loading dose on Day 1, Week 1, and Week 2, followed by Q4W starting at Week 4

The planned duration of the Treatment Period 1 is 16 weeks. All study drug injections will be administered at the clinic site during this treatment period. The planned End of Treatment Period 1 visit (EOT 1) will be performed at Week 16. Participants who complete Treatment Period 1 will continue in Treatment Period 2.

### Treatment Period 2 (Double-Blind, 36 Weeks)

Participants who have completed the Week 16 visit will continue in the Treatment Period 2.
Participants who were randomized to 600 mg Q4W regimen in TP1 will receive the same dose either Q4W or Q12W (1:1 ratio) (s.c.)
Participants who were randomized to 300 mg Q4W regimen in TP1 will receive 300 mg Q12W (s.c.)
Participants who were randomized to placebo arm, 60 mg Q4W, and 150 mg Q4W in TP1 will receive 600 mg Q4W with loading doses of 600 mg (administered at Week 16, 17, and 18) (s.c.) (for the antibody dosing cohorts, if they are classified as non-responders after completion of TP1).
Participants who were randomized to 60 mg Q4W and 150 mg Q4W in TP1, and are classified as responders at the end of TP1, will continue with the same dosing regimen in TP2 as they received in TP1.

A participant is considered a responder if they meet IGA criteria of either “0 (Clear)” or “1 (Almost clear)” and at least 2 points reduction from baseline IGA response at Week 16. Participants who do not achieve an IGA 0 or 1 score and at least 2 points reduction from baseline IGA response at Week 16 are considered non-responders to the antibody treatment. The responder status at Week 16 will be assessed for all participants across all treatment arms.

All study drug injections will be administered at the clinic site during this treatment period. Sham dosing will be administered to maintain the blinding and minimize bias. The planned end of Treatment Period 2 visit (EOT 2) will be performed at Week 52.

Follow-up period (52 weeks): No study treatment will be given during the safety follow-up period. Safety and periodic assessments for off-treatment efficacy will be assessed during this period. A summary of the study design is provided in FIG. 6.

1.-73. (canceled)

74. A method of treatment and/or prevention of atopic dermatitis in a human subject in need thereof, comprising administering at least one loading dose and at least one maintenance dose of a multispecific antibody or antigen-binding fragment thereof to the subject, wherein the antibody comprises:
i) a first part that binds to IL-18 comprising:
an HCDR1 comprising SEQ ID NO: 29, SEQ ID NO: 32, SEQ ID NO: 35, or SEQ ID NO: 38;
an HCDR2 comprising SEQ ID NO: 30, SEQ ID NO: 33, SEQ ID NO: 36, or SEQ ID NO: 39;
an HCDR3 comprising SEQ ID NO: 31, SEQ ID NO: 34, SEQ ID NO: 37, or SEQ ID NO: 40;
an LCDR1 comprising SEQ ID NO: 1, SEQ ID NO: 4, SEQ ID NO: 7, or SEQ ID NO: 10;
an LCDR2 comprising SEQ ID NO: 2, SEQ ID NO: 5, or amino acid sequence RNN, and;
an LCDR3 comprising SEQ ID NO: 3, SEQ ID NO: 6, SEQ ID NO: 9, or SEQ ID NO: 12, and;
ii) a second part that binds to IL-13 comprising:
an HCDR1 comprising SEQ ID NO: 43, SEQ ID NO: 46, SEQ ID NO: 49, or SEQ ID NO: 52;
an HCDR2 comprising SEQ ID NO: 44, SEQ ID NO: 47, SEQ ID NO: 50, or SEQ ID NO: 53;
an HCDR3 comprising SEQ ID NO: 45, SEQ ID NO: 48, SEQ ID NO: 51, or SEQ ID NO: 54;
an LCDR1 comprising SEQ ID NO: 15, SEQ ID NO: 18, SEQ ID NO: 21, or SEQ ID NO: 24;
an LCDR2 comprising SEQ ID NO: 16, SEQ ID NO: 19, or amino acid sequence LAS, and;
an LCDR3 comprising SEQ ID NO: 17, SEQ ID NO: 20, SEQ ID NO: 23, or SEQ ID NO: 26.

75. The method of claim 74, wherein the antibody is bispecific.

76. The method of claim 74, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

77. The method of claim 74, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

78. The method of claim 74, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

79. The method of claim 74, wherein the at least one loading dose and/or the at least one maintenance dose is administered subcutaneously or intravenously.

80. The method of claim 74, wherein the at least one loading dose and/or the at least one maintenance dose comprise an amount of the multispecific antibody or antigen-binding fragment thereof sufficient to achieve a therapeutically effective serum level.

81. The method of claim 80, wherein the therapeutically effective serum level is maintained throughout the course of the treatment.

82. The method of claim 74, wherein the at least one loading dose and/or at least one maintenance dose comprises an amount of the multispecific antibody or antigen-binding fragment thereof selected from the group consisting of about 60 mg, about 150 mg, about 300 mg, and about 600 mg.

83. The method of claim 82, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

84. The method of claim 74, wherein at least one, at least two, or at least three (e.g., three) loading doses comprising about 150 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 150 mg of the multispecific antibody or antigen-binding fragment thereof are administered.

85. The method of claim 84, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 150 mg of the multispecific antibody or antigen-binding fragment thereof.

86. The method of claim 85, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

87. The method of claim 85, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

88. The method of claim 74, wherein at least one, at least two, or at least three (e.g., three) loading doses comprising about 300 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 300 mg of the multispecific antibody or antigen-binding fragment thereof are administered.

89. The method of claim 88, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 300 mg of the multispecific antibody or antigen-binding fragment thereof.

90. The method of claim 89, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

91. The method of claim 89, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

92. The method of claim 74, wherein at least one, at least two, or at least three (e.g., three) loading doses comprising about 600 mg of the multispecific antibody or antigen-binding fragment thereof, and at least one, at least two, or at least three (e.g., at least two, at least three, at least four, at least five, at least six, at least seven, at least eight, at least nine, at least ten, at least eleven, or at least twelve) maintenance doses comprising about 600 mg of the multispecific antibody or antigen-binding fragment thereof are administered.

93. The method of claim 92, wherein:
a first loading dose is administered on day 1, a second loading dose is administered on week 1, and a third loading dose is administered on week 2 of the treatment;
a maintenance dose is administered on week 4, followed by administration once every two weeks (Q2W) or once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter,
wherein each loading dose and maintenance dose comprises about 600 mg of the multispecific antibody or antigen-binding fragment thereof.

94. The method of claim 93, wherein:
a maintenance dose is administered on week 4, followed by administration once every two weeks (Q2W) until week 12 or week 16 of the treatment (or until a positive response to the treatment is obtained), optionally followed by administration once every four weeks (Q4W), once every eight weeks (Q8W), or once every twelve weeks (Q12W) thereafter.

95. The method of claim 93, wherein:
a maintenance dose is administered on week 4, followed by administration once every four weeks (Q4W) until week 12 or week 16 of the treatment (or until a positive response to the treatment is obtained), optionally followed by administration once every four weeks (Q4W), once every eight weeks (Q8W), or once every twelve weeks (Q12W) thereafter.

96. The method of claim 74, wherein the treatment results in an improvement relative to baseline in an atopic dermatitis-associated parameter selected from the group consisting of:
a decrease in Investigator's Global Assessment (IGA) score by at least 2 points, preferably a decrease in IGA score by at least by 2 points and achieving clear or almost clear status;
a decrease of ≥50% (EASI-50), ≥75% (EASI-75), ≥90% (EASI-90), or 100% (EASI-100%) in Eczema Area and Severity Index (EASI) score;
achieving 0-1 score band or 2-5 score band response in Dermatology Life Quality Index (DLQI) score;
a decrease of ≥3-points or ≥4-points in Peak Pruritus Numeric Rating Scale (PP-NRS) score, and;
a combination thereof,
optionally obtained at or before week 16 or week 52 of the treatment.

97. The method of claim 74, wherein the multispecific antibody or antigen-binding fragment thereof is comprised in a pharmaceutical composition, wherein the pharmaceutical composition optionally comprises one or more pharmaceutically acceptable ingredients.

98. A method of treatment and/or prevention of atopic dermatitis in a human subject in need thereof, wherein the subject has received at least one dose of a multispecific antibody or antigen-binding fragment thereof, comprising administering at least one maintenance dose of the multispecific antibody or antigen-binding fragment thereof to the subject, wherein the antibody comprises:
i) a first part that binds to IL-18 comprising:
an HCDR1 comprising SEQ ID NO: 29, SEQ ID NO: 32, SEQ ID NO: 35, or SEQ ID NO: 38;
an HCDR2 comprising SEQ ID NO: 30, SEQ ID NO: 33, SEQ ID NO: 36, or SEQ ID NO: 39;
an HCDR3 comprising SEQ ID NO: 31, SEQ ID NO: 34, SEQ ID NO: 37, or SEQ ID NO: 40;
an LCDR1 comprising SEQ ID NO: 1, SEQ ID NO: 4, SEQ ID NO: 7, or SEQ ID NO: 10;
an LCDR2 comprising SEQ ID NO: 2, SEQ ID NO: 5, or amino acid sequence RNN, and;
an LCDR3 comprising SEQ ID NO: 3, SEQ ID NO: 6, SEQ ID NO: 9, or SEQ ID NO: 12, and;
ii) a second part that binds to IL-13 comprising:
an HCDR1 comprising SEQ ID NO: 43, SEQ ID NO: 46, SEQ ID NO: 49, or SEQ ID NO: 52;
an HCDR2 comprising SEQ ID NO: 44, SEQ ID NO: 47, SEQ ID NO: 50, or SEQ ID NO: 53;
an HCDR3 comprising SEQ ID NO: 45, SEQ ID NO: 48, SEQ ID NO: 51, or SEQ ID NO: 54;
an LCDR1 comprising SEQ ID NO: 15, SEQ ID NO: 18, SEQ ID NO: 21, or SEQ ID NO: 24;
an LCDR2 comprising SEQ ID NO: 16, SEQ ID NO: 19, or amino acid sequence LAS, and;
an LCDR3 comprising SEQ ID NO: 17, SEQ ID NO: 20, SEQ ID NO: 23, or SEQ ID NO: 26.

99. The method of claim 98, wherein the antibody is bispecific.

100. The method of claim 98, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

101. The method of claim 98, wherein the antibody is bispecific and comprises:
i) a first part that binds to IL-18 comprising a first heavy chain (HC1) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

102. The method of claim 98, wherein the antibody is bispecific and comprises:
i) a first part that binds to IL-18 comprising a first heavy chain (HC1) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

103. The method of claim 98, wherein the atopic dermatitis is moderate-to-severe atopic dermatitis.

104. The method of claim 98, wherein the at least one maintenance dose is administered subcutaneously or intravenously.

105. The method of claim 98, wherein the at least one maintenance dose comprises an amount of the multispecific antibody or antigen-binding fragment thereof selected from the group consisting of about 60 mg, about 150 mg, about 300 mg, and about 600 mg.

106. The method of claim 105, wherein maintenance dose administration frequency is selected from the group consisting of once a week (Q1W), once every two weeks (Q2W), once every four weeks (Q4W), one every eight weeks (Q8W), once every twelve weeks (Q12W), and a combination thereof.

107. The method of claim 106, wherein the treatment has a total duration selected from the group consisting of at least four weeks, at least eight weeks, at least twelve weeks, at least sixteen weeks, at least twenty weeks, at least twenty-four weeks, at least twenty-eight weeks, at least thirty-two weeks, at least thirty-six weeks, at least forty weeks, at least forty-four weeks, at least forty-eight weeks, and at least fifty-two weeks.

108. The method of claim 98, wherein a maintenance dose is administered two weeks, four weeks, eight weeks, or twelve weeks after the last dose has been administered to the subject.


SEQUENCE LISTING (62 sequences)

SEQ ID NO: 1 - protein, 13 aa, Synthetic construct
  source 1..13: note = LCDR1
SGSSSNIGNHYVN

SEQ ID NO: 2 - protein, 7 aa, Synthetic construct
  source 1..7: note = LCDR2
RNNHRPS

SEQ ID NO: 3 - protein, 11 aa, Synthetic construct
  source 1..11: note = LCDR3
QSWDYSGFSTV

SEQ ID NO: 4 - protein, 13 aa, Synthetic construct
  source 1..13: note = LCDR1
SGSSSNIGNHYVN

SEQ ID NO: 5 - protein, 7 aa, Synthetic construct
  source 1..7: note = LCDR2
RNNHRPS

SEQ ID NO: 6 - protein, 11 aa, Synthetic construct
  source 1..11: note = LCDR3
QSWDYSGFSTV

SEQ ID NO: 7 - protein, 9 aa, Synthetic construct
  source 1..9: note = LCDR1
SSSNIGNHY

SEQ ID NO: 8 - skipped

SEQ ID NO: 9 - protein, 8 aa, Synthetic construct
  source 1..8: note = LCDR3
WDYSGFST

SEQ ID NO: 10 - protein, 8 aa, Synthetic construct
  source 1..8: note = LCDR1
SSNIGNHY

SEQ ID NO: 11 - skipped

SEQ ID NO: 12 - protein, 11 aa, Synthetic construct
  source 1..11: note = LCDR3
QSWDYSGFSTV

SEQ ID NO: 13 - protein, 110 aa, Synthetic construct
  source 1..110: note = VL
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVL

SEQ ID NO: 14 - protein, 216 aa, Synthetic construct
  source 1..216: note = Light chain 1
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 15 aa, Synthetic construct
  source 1..15: note = LCDR1
RASKSVDSYGNSFMH

SEQ ID NO: 16 - protein, 7 aa, Synthetic construct
  source 1..7: note = LCDR2
LASNLES

SEQ ID NO: 17 - protein, 9 aa, Synthetic construct
  source 1..9: note = LCDR3
QQNNEDPRT

SEQ ID NO: 18 - protein, 15 aa, Synthetic construct
  source 1..15: note = LCDR1
RASKSVDSYGNSFMH

SEQ ID NO: 19 - protein, 7 aa, Synthetic construct
  source 1..7: note = LCDR2
LASNLES

SEQ ID NO: 20 - protein, 9 aa, Synthetic construct
  source 1..9: note = LCDR3
QQNNEDPRT

SEQ ID NO: 21 - protein, 11 aa, Synthetic construct
  source 1..11: note = LCDR1
SKSVDSYGNSF

SEQ ID NO: 22 - skipped

SEQ ID NO: 23 - protein, 6 aa, Synthetic construct
  source 1..6: note = LCDR3
NNEDPR

SEQ ID NO: 24 - protein, 10 aa, Synthetic construct
  source 1..10: note = LCDR1
KSVDSYGNSF

SEQ ID NO: 25 - skipped

SEQ ID NO: 26 - protein, 9 aa, Synthetic construct
  source 1..9: note = LCDR3
QQNNEDPRT

SEQ ID NO: 27 - protein, 111 aa, Synthetic construct [sequence omitted]

SEQ ID NO: 28 - protein, 218 aa, Synthetic construct
  source 1..218: note = Light chain 2
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 10 aa, Synthetic construct
  source 1..10: note = HCDR1
GGTFKSYAIS

SEQ ID NO: 30 - protein, 17 aa, Synthetic construct
  source 1..17: note = HCDR2
NIIPMTGQTYYAQKFQG

SEQ ID NO: 31 - protein, 10 aa, Synthetic construct
  source 1..10: note = HCDR3
AAYHPLVFDN

SEQ ID NO: 32 - protein, 5 aa, Synthetic construct
  source 1..5: note = HCDR1
SYAIS

SEQ ID NO: 33 - protein, 17 aa, Synthetic construct
  source 1..17: note = HCDR2
NIIPMTGQTYYAQKFQG

SEQ ID NO: 34 - protein, 10 aa, Synthetic construct
  source 1..10: note = HCDR3
AAYHPLVFDN

SEQ ID NO: 35 - protein, 7 aa, Synthetic construct
  source 1..7: note = HCDR1
GGTFKSY

SEQ ID NO: 36 - protein, 6 aa, Synthetic construct
  source 1..6: note = HCDR2
IPMTGQ

SEQ ID NO: 37 - protein, 10 aa, Synthetic construct
  source 1..10: note = HCDR3
AAYHPLVFDN

SEQ ID NO: 38 - protein, 8 aa, Synthetic construct
  source 1..8: note = HCDR1
GGTFKSYA

SEQ ID NO: 39 - protein, 8 aa, Synthetic construct
  source 1..8: note = HCDR2
IIPMTGQT

SEQ ID NO: 40 - protein, 12 aa, Synthetic construct
  source 1..12: note = HCDR3
ARAAYHPLVFDN

SEQ ID NO: 41 - protein, 119 aa, Synthetic construct
  source 1..119: note = VH
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSS

SEQ ID NO: 42 - protein, 449 aa, Synthetic construct
  source 1..449: note = Heavy chain 1
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 10 aa, Synthetic construct
  source 1..10: note = HCDR1
GFSLSAYSVN

SEQ ID NO: 44 - protein, 16 aa, Synthetic construct
  source 1..16: note = HCDR2
MIWGDGKIVYNSALKS

SEQ ID NO: 45 - protein, 10 aa, Synthetic construct
  source 1..10: note = HCDR3
DGYYPYAMDN

SEQ ID NO: 46 - protein, 5 aa, Synthetic construct
  source 1..5: note = HCDR1
AYSVN

SEQ ID NO: 47 - protein, 16 aa, Synthetic construct
  source 1..16: note = HCDR2
MIWGDGKIVYNSALKS

SEQ ID NO: 48 - protein, 10 aa, Synthetic construct
  source 1..10: note = HCDR3
DGYYPYAMDN

SEQ ID NO: 49 - protein, 7 aa, Synthetic construct
  source 1..7: note = HCDR1
GFSLSAY

SEQ ID NO: 50 - protein, 5 aa, Synthetic construct
  source 1..5: note = HCDR2
WGDGK

SEQ ID NO: 51 - protein, 10 aa, Synthetic construct
  source 1..10: note = HCDR3
DGYYPYAMDN

SEQ ID NO: 52 - protein, 8 aa, Synthetic construct
  source 1..8: note = HCDR1
GFSLSAYS

SEQ ID NO: 53 - protein, 7 aa, Synthetic construct
  source 1..7: note = HCDR2
IWGDGKI

SEQ ID NO: 54 - protein, 12 aa, Synthetic construct
  source 1..12: note = HCDR3
AGDGYYPYAMDN

SEQ ID NO: 55 - protein, 118 aa, Synthetic construct [sequence omitted]

SEQ ID NO: 56 - protein, 448 aa, Synthetic construct
  source 1..448: note = Heavy chain 2
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 449 aa, Synthetic construct
  source 1..449: note = Heavy chain 1
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 448 aa, Synthetic construct
  source 1..448: note = Heavy chain 2
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 227 aa, Synthetic construct
  source 1..227: note = Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, Synthetic construct
  source 1..227: note = Fc-hole
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 227 aa, Synthetic construct
  source 1..227: note = Fc-key
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 193 aa, Synthetic construct
  source 1..193: note = IL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED